(19) United States
(12) Patent Application Publication
(10) Pub. No.: US 2013/0004979 A1
(43) Pub. Date: Jan. 3, 2013

(54) GLYCOSYLTRANSFERASE REVERSIBILITY FOR SUGAR NUCLEOTIDE SYNTHESIS AND MICROSCALE SCANNING

(75) Inventors: Jon S. Thorson, Lexington, KY (US); Richard W. Gantt, Roswell, GA (US); Pauline Marie Jeanne Peltier-Pain, Orleans (FR)

(73) Assignee: WISCONSIN ALUMNI RESEARCH FOUNDATION, Madison, WI (US)

(21) Appl. No.: 13/495,760

(22) Filed: Jun. 13, 2012

Related U.S. Application Data
(63) Continuation-in-part of application No. 13/159,097, filed on Jun. 13, 2011.
(60) Provisional application No. 61/496,239, filed on Jun. 13, 2011.
(60) Provisional application No. 61/354,037, filed on Jun. 11, 2010.

(51) Int. Cl. C12N 9/10; C12P 21/00; G01N 21/64; C12N 15/70; C12N 1/21; C12P 19/30; C12N 15/54

(52) U.S. Cl. 435/15; 435/193; 435/89; 435/68.1; 536/23.2; 435/320.1; 435/252.33

(57) ABSTRACT

The present invention generally relates to materials and methods for exploiting glycosyltransferase reversibility for nucleotide diphosphate (NDP) sugar synthesis. The present invention provides engineered glycosyltransferase enzymes characterized by improved reaction reversibility and expanded sugar donor specificity as compared to corresponding non-mutated glycosyltransferase enzymes. Such reagents provide advantageous routes to NDP sugars for subsequent use in a variety of biomedical applications, including enzymatic and chemo-enzymatic glycorandomization.

### CROSS-REFERENCE TO RELATED APPLICATIONS

This application claims the benefit of U.S. Provisional Patent Application No. 61/496,239, filed Jun. 13, 2011, and is a continuation-in-part of U.S. patent application Ser. No. 13/159,097, filed Jun. 13, 2011, which claims the benefit of U.S. Provisional Application No. 61/354,037, filed Jun. 11, 2010, the entirety of each hereby incorporated by reference herein for all purposes.

### STATEMENT RELATED TO FEDERAL FUNDING

This invention was made with government support under Grant No. Al052218, awarded by the National Institutes of Health. The government has certain rights in the invention.

### FIELD OF THE INVENTION

This invention relates generally to the fields of glycobiology and the synthesis of glycosylated compounds. In particular, the present invention encompasses materials and methods for exploiting glycosyltransferase reversibility to provide nucleotide diphosphate (NDP) sugar synthesis.

### BACKGROUND OF THE INVENTION

Glycosyltransferases (GTs) constitute a large family with approximately 23,000 predicted or known GT sequences in the CAZY database divided into 87 families based upon amino acid similarity. Despite the vast range of GT sugar donors and acceptors (sugars, proteins, nucleic acids, lipids, and small molecules), GTs are generally classified into two simple groups based upon mechanism (inverting or retaining), and primarily fall within two main structural superfamilies (GT-A and GT-B). Lairson L L, et al. (2004) Chem Commun 2243-8; Hu Y., et al. (2002) Chem Biol 9: 1287-96. The GT-B fold is the predominate fold of natural product GTs and is characterized by two closely associated Rossman-like domains, each of which is usually distinguished as the acceptor- and donor-binding domains (N and C-terminal domains, respectively). Despite the wealth of GT structural and biochemical information, attempts to alter GT donor/acceptor specificities via rational engineering have been largely unsuccessful and primarily limited to sequence-guided single site mutagenesis. Hancock S M, et al. (2006) Curr Opin Chem Biol 10: 509-19. While there exists precedent for the directed evolution of carbohydrate-utilizing enzymes, the lack of sensitive high-throughput screens for GTs has also hampered GT directed evolution. Hoffmeister D, et al. (2003) Proc Natl Acad Sci USA 100: 13184-9; Williams al, et al. (2006) J Am Chem Soc 128: 16238-47.

Nucleotide diphosphate (NDP) sugars are common substrates for GTs where they routinely act as glycoside donors. A generic structure for an NDP sugar is depicted in FIG. 1. In general, NDP sugars represent a class of compounds routinely utilized in the investigation of polysaccharide formation for basic metabolism, intra- and extracellular transport, cell wall biosynthesis within virulent organisms, and drug discovery. However, synthesis of sugar-nucleotides is currently expensive, difficult and time-consuming, and is further complicated by their low solubility in organic solvents and susceptibility to both chemical and enzymatic hydrolysis. Further, an exemplary GT reaction utilizing an NDP sugar, in this case GtfD, is shown in FIG. 2.

While classical synthetic strategies to access sugar nucleotides are available, most require many steps and often suffer from low-yielding reactions, difficult purifications, and a lack of stereochemical control. Accordingly, a need exists for new reagents and routes to provide NDP sugars for a variety of uses in the biomedical field.

### SUMMARY OF THE INVENTION

The present invention relates to novel glycosyltransferases and improved methods of NDP-sugar synthesis. Applications for this novel method include efficient synthesis of NDP-sugars with complete stereochemical control, in vitro formation for drug discovery, and robust microscale glycosyl scanning for assessing large compound libraries.

Accordingly, the invention provides in a first aspect an isolated mutant glycosyltransferase comprising: (a) the amino acid sequence of OleD glycosyltransferase set forth in SEQ ID NO:1, wherein proline at position 67 has been replaced with threonine, serine at position 132 has been replaced with phenylalanine, alanine at position 242 has been replaced with leucine, and glutamine at position 268 has been replaced with valine; or (b) an amino acid sequence substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine; wherein the isolated mutant exhibits an improved conversion of nucleotide diphosphate (NDP) to NDP sugar as compared to a corresponding non-mutated glycosyltransferase. In preferred embodiments, the isolated mutant glycosyltransferase is encoded by a nucleotide that hybridizes under stringent conditions to the nucleotide sequence set forth in SEQ ID NO:2.

In a second aspect, the invention provides a method of providing an isolated mutant glycosyltransferase with improved conversion of nucleotide diphosphate (NDP) to NDP sugar as compared to a corresponding non-mutated glycosyltransferase. Such a method includes steps of: (a) mutating an isolated nucleic acid sequence encoding an amino acid sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine; (b) expressing said isolated nucleic acid in a host cell; and (c) isolating from the host cell a mutant glycosyltransferase that is characterized by improved conversion of nucleotide diphosphate (NDP) to NDP sugar as compared to a corresponding non-mutated glycosyltransferase.

In another aspect, the invention encompasses a method of providing a nucleotide diphosphate (NDP) sugar. Such a method includes steps of incubating a nucleotide diphosphate and a glycoside donor in the presence of an isolated mutant glycosyltransferase described and claimed herein to provide an NDP sugar.

In certain methods, the glycoside donor has the structure:

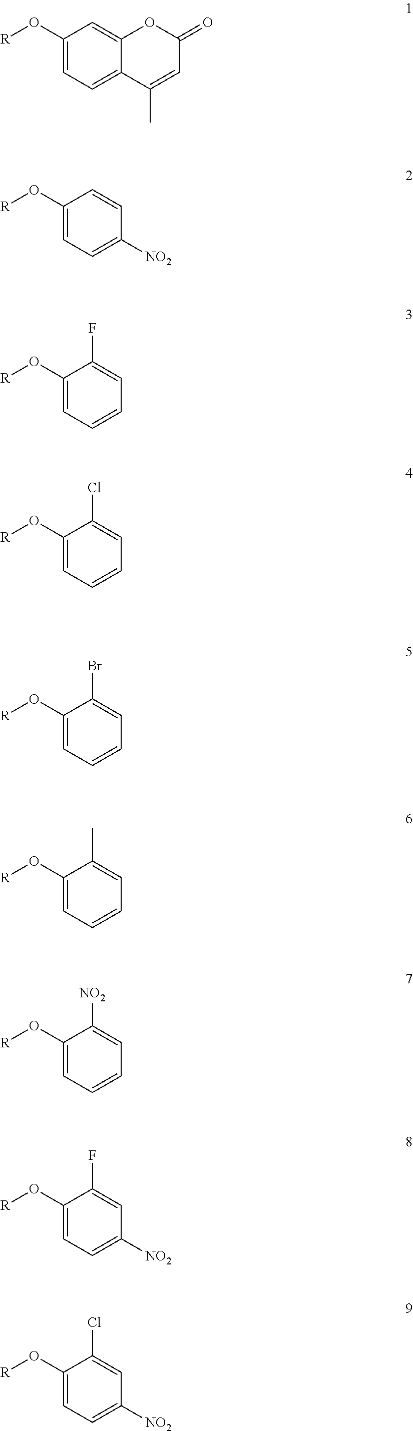

wherein R is β-D-glucopyranose.

In other embodiments, the glycoside donor has the structure:

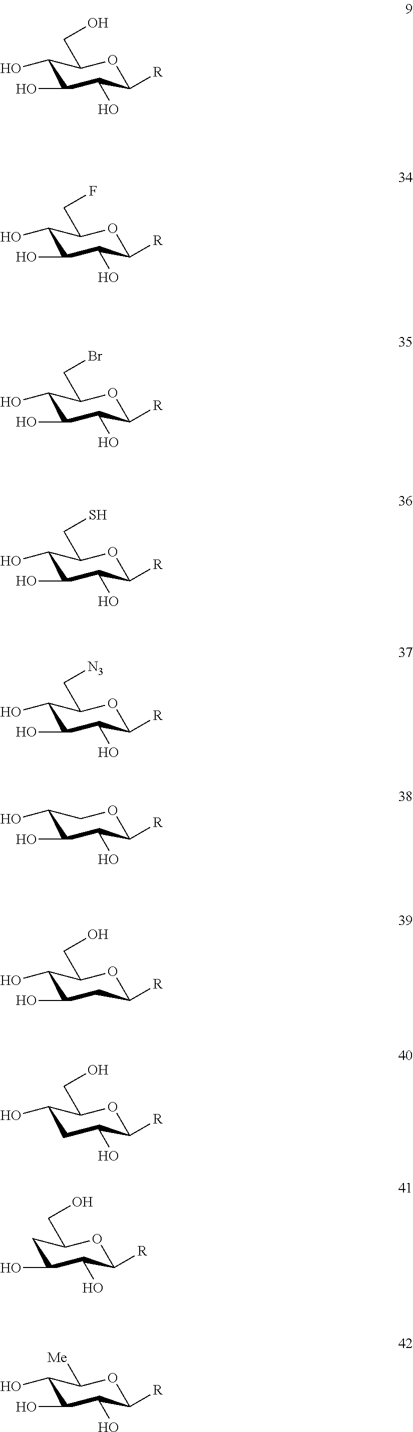

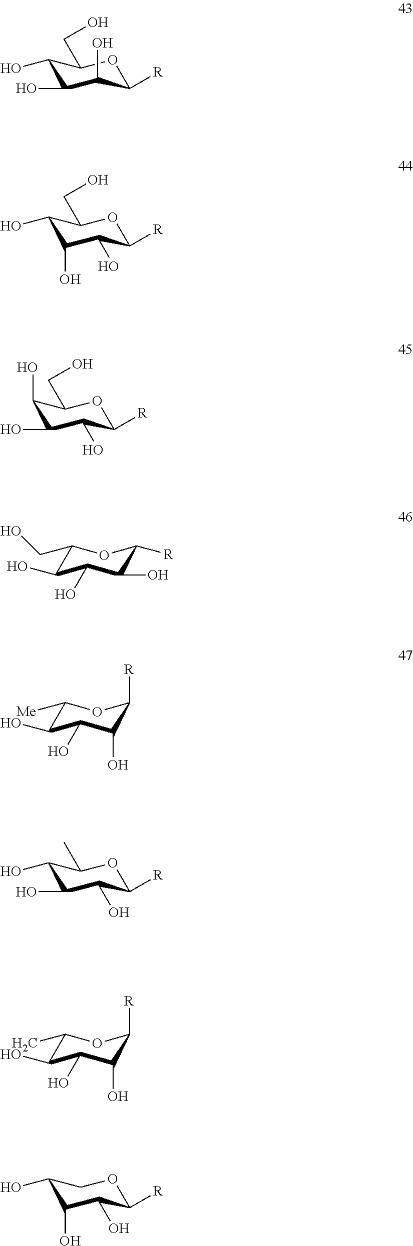

wherein R is:

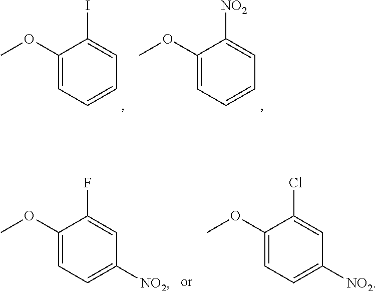

The NDP is preferably uridine or thymidine diphosphate. In alternative embodiments, the NDP sugar includes a ¹³C atom. Such labeled compounds are particularly useful in bioimaging studies, particularly nuclear magnetic resonance (NMR) studies.

Yet another aspect of the invention is directed to a method of providing a glycosylated target molecule. Such a method includes steps of: (a) incubating a nucleotide diphosphate and a glycoside donor in the presence of an isolated mutant glycosyltransferase as described and claimed herein to provide a nucleotide diphosphate (NDP) sugar; and (b) further incubating the NDP sugar with a second glycosyltransferase and a target molecule to provide a glycosylated target molecule.

In certain embodiments, the glycoside donor has the structure:

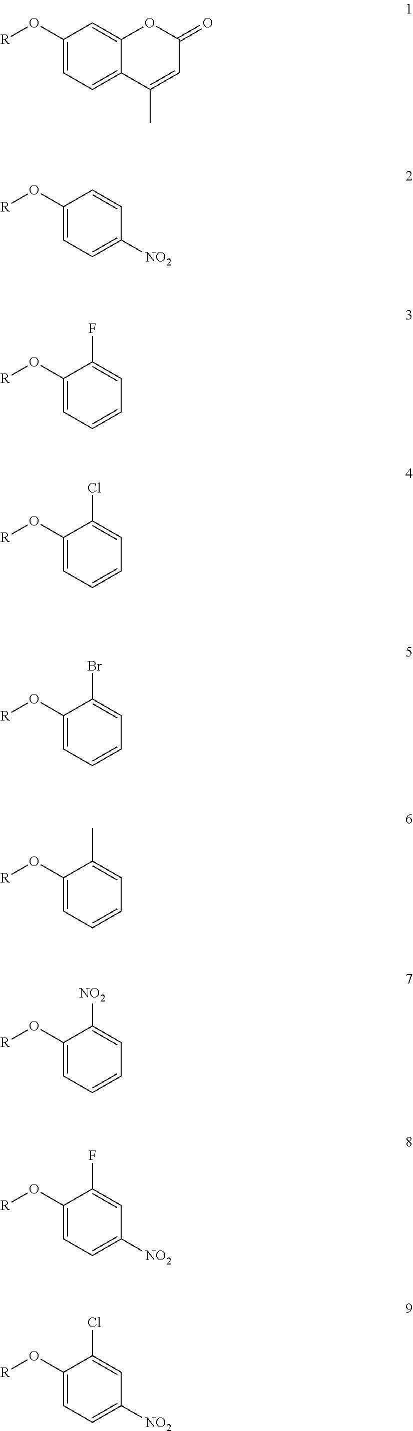

wherein R is β-D-glucopyranose.

In other embodiments, the glycoside donor has the structure:

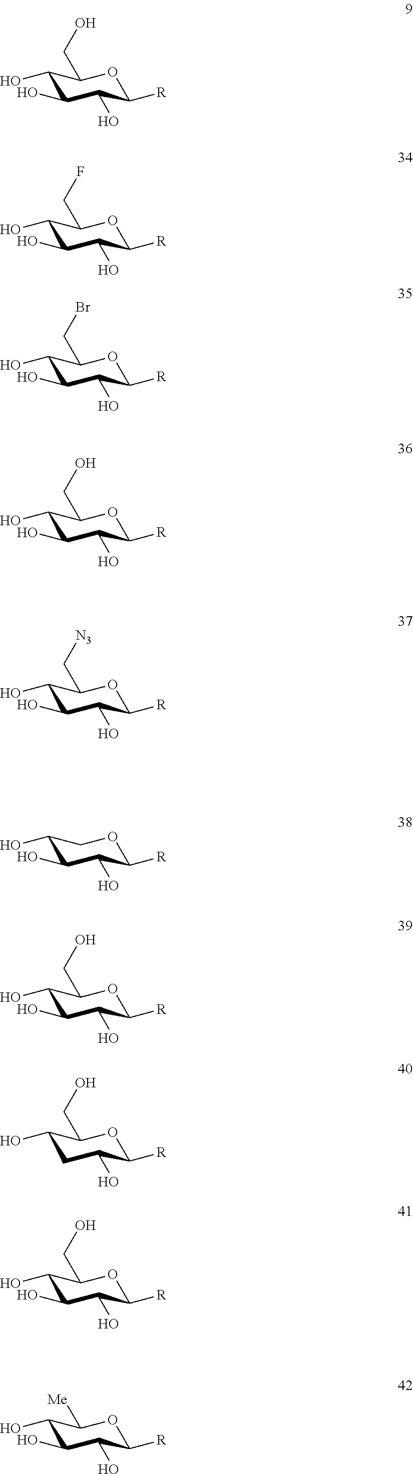

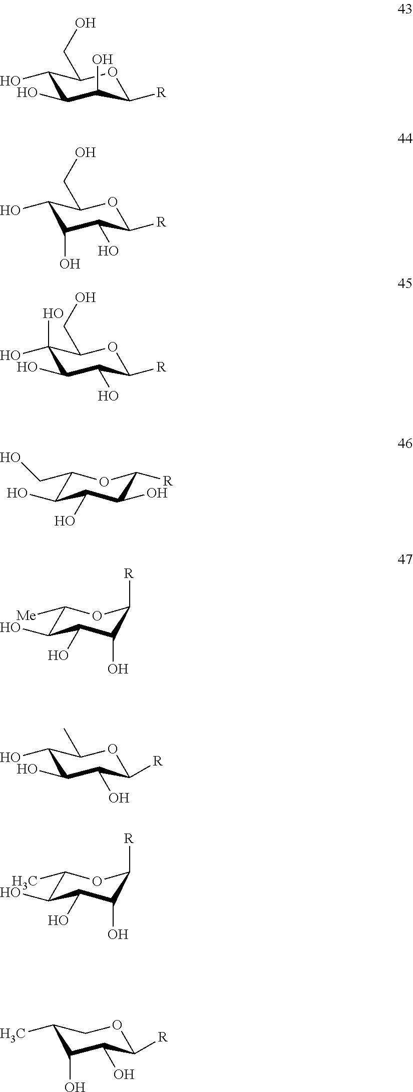

wherein R is:

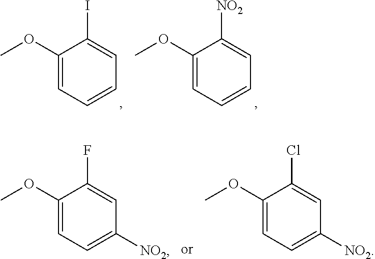

Suitable target molecules for use in the present method include, but are not limited to, natural or synthetic pyran rings, furan rings, enediynes, anthracyclines, angucyclines, aureolic acids, orthosomycins, macrolides, aminoglycosides, non-ribosomal peptides, polyenes, steroids, lipids, indolocarbazoles, bleomycins, amicetins, benzoisochromanequinones, flavonoids, isoflavones, coumarins, aminocoumarins, coumarin acids, polyketides, pluramycins, aminoglycosides, oligosaccharides, nucleosides, peptides and proteins.

In alternative embodiments, the method is carried out in vitro, preferably in a single reaction vessel.

In other embodiments, more than one type of target molecule is incubated with the second glycosyltransferase to produce a diverse population of glycosylated target molecules. As well, more than one type of NDP may be incubated with the isolated mutant glycosyltransferase to produce a diverse population of NDP sugars.

The invention further provides an isolated nucleic acid encoding a mutant glycosyltransferase having a polypeptide sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine, wherein the isolated mutant glycosyltransferase exhibits an improved conversion of nucleotide diphosphate (NDP) to NDP sugar as compared to a corresponding non-mutated glycosyltransferase.

In a preferred embodiment, the isolated nucleic acid hybridizes under stringent conditions to the nucleotide sequence set forth in SEQ ID NO:2.

In various related aspects, a recombinant vector comprising the isolated nucleic acid and a host cell comprising same are provided by the invention.

Another aspect of the invention is a fluorescent-based assay for identifying a mutant glycosyltransferase exhibiting an improved conversion of nucleotide diphosphate (NDP) to NDP sugar as compared to a corresponding non-mutated glycosyltransferase. Such a method includes steps of: (a) providing a mutant glycosyltransferase; (b) incubating the mutant glycosyltransferase with an NDP and a fluorescent glycoside donor; and (c) measuring a change in fluorescence intensity of the fluorescent glycoside donor incubated with the mutant glyscosyltransferase, the mutant glycosyltransferase's ability to transfer a sugar from said fluorescent glycoside donor to the NDP to form an NDP sugar indicated by an increase in the fluorescence of the fluorescent glycoside donor incubated with the mutant glycosyltransferase; wherein the mutant glycosyltransferase exhibits an improved conversion of NDP to NDP sugar by displaying an increase in the fluorescent glycoside donor fluorescence as compared to a corresponding non-mutated glycosyltransferase.

In certain embodiments, the glycoside donor has the structure:

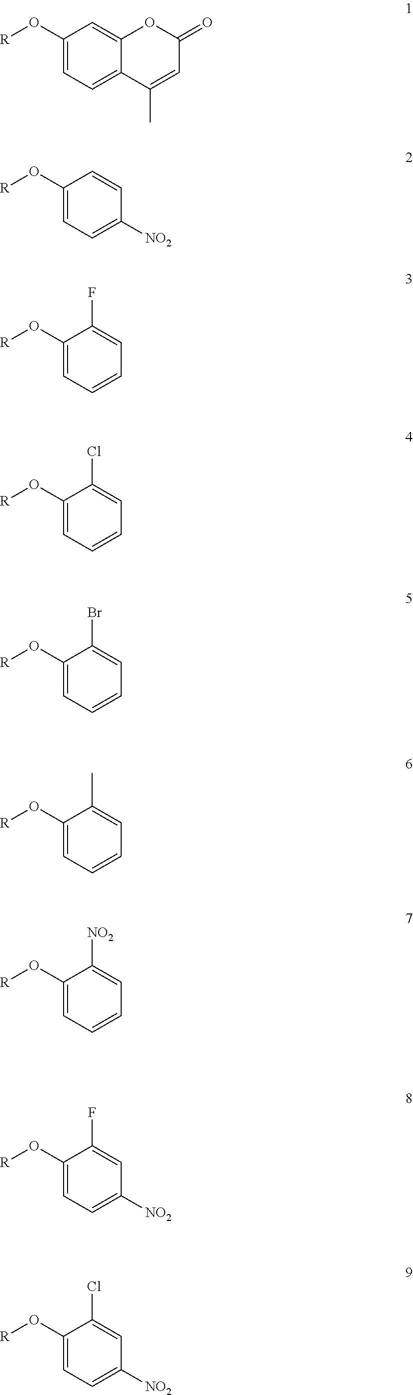

wherein R is β-D-glucopyranose.

In other embodiments, the glycoside donor has the structure:

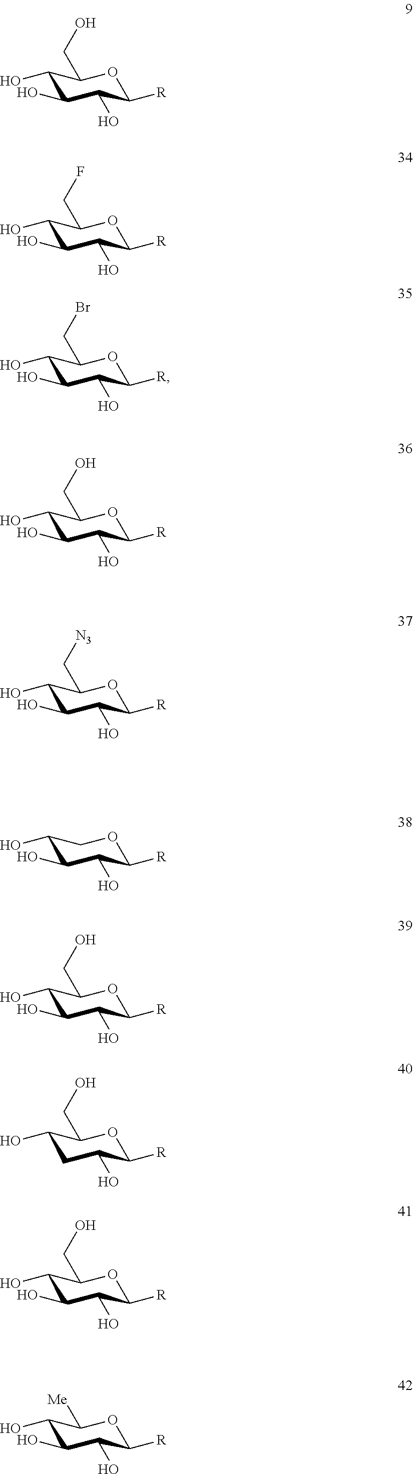

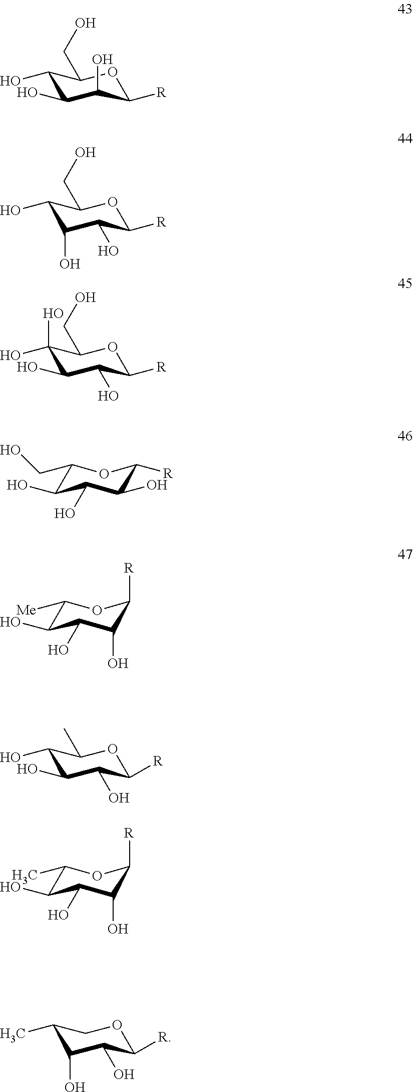

wherein R is:

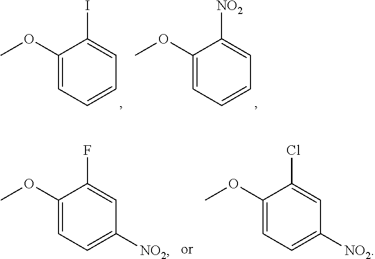

In preferred embodiments, the assay is carried out in parallel on a plurality of mutant glycosyltransferases.

In yet another exemplary embodiment, the present invention provides a method of generating a library of novel NDP sugars. For instance, in one example of the present invention, milligram quantities of fully characterized NDP sugars were prepared rapidly and efficiently. Specifically, 22 different sugars were generated in a matter of hours.

The present invention provides novel glycosyltransferase and methods of preparing NDP-sugars, and will be useful for preparing milligram scale NDP-sugar libraries for biochemical investigations and drug discovery. In addition, the novel glycosyltransferase and methods of preparing NDP-sugars of the present invention will be useful for preparing ¹³C labeled NDP-sugars for biosynthetic investigations by NMR, protein engineering and evolution, coupled reactions to prepare, for example, a specific glycosylated target compound.

In another aspect, the novel glycosyltransferase and methods of preparing NDP-sugars of the present invention are useful for microscale glycosyl scanning to provide a rapid means of assessing glycosylation of large compound libraries. For instance, newly identified glycosyltransferases can be screened for activity toward specific aglycons or sugars. Once identified, compounds can be further diversified via enzymatic or chemoselective glycosylation.

In certain embodiments, methods according to the invention utilize simple glycoside donors which dramatically shift the equilibrium of the reaction so that the reverse reaction is favored (even at sub-stoichiometric amounts of NDP). This drives coupled reactions (e.g., the microscale reaction) by immediately converting NDP produced upon glycosyl transfer back to NDP-sugar. As a result, it also prevents feedback inhibition by NDP.

Other objects, features and advantages of the present invention will become apparent after review of the specification, claims and drawings.

### BRIEF DESCRIPTION OF DRAWINGS

FIG. 1 depicts the general structure of a nucleotide diphosphate (NDP) sugar.

FIG. 2 shows the reaction catalyzed by GtfD which utilizes an NDP sugar as a donor substrate.

FIG. 3 illustrates glycosyltransferase (GT) reversibility and the utilization of a synthetic glycoside donor.

FIG. 4 depicts the reaction catalyzed by wild type OleD. OleD was mutated to accept three different nitrogenous bases, approximately 20 sugars, and approximately 90 aglycons.

FIG. 5 illustrates the inventors' strategy and results for β-D-glucoside screening of OleD variants.

FIG. 6 shows the inventors' identification of OleD variant P67T/S132F/A242L/Q268V and optimization of reverse reaction conditions.

FIG. 7 depicts the inventors' strategy and results for glycoside library evaluation.

FIG. 8 depicts the inventors' conclusions for structure activity relationship (SAR) evaluation of NDP sugar substrates related to OleD variant substrate usage.

FIG. 9 illustrates a general scheme for production of milligram scale NDP-sugar libraries for biochemical investigations and drug discovery.

FIG. 10 depicts production of ¹³C Labeled NDP-sugars for bioimaging studies.

FIG. 11 depicts a general strategy for protein engineering and evolution based on the OleD variants.

FIG. 12 depicts the coupling of GT reactions to initially provide an NDP sugar which serves as the glycoside donor in a second GT reaction to provide a glycosylated target.

FIG. 13 illustrates an approach to microscale glycosyl scanning according to the invention.

FIG. 14. Evaluation of putative donors for sugar nucleotide synthesis. (a) General reaction scheme. (b) Structures of the 3-D-glucopyranoside donors which led to (U/T)DP-glucose formation. (c) Percent conversion of (U/T)DP to (U/T)DP-glucose with various donors. Reactions contained 7 μM OleD variant, 1 mM of (U/T)DP, and 1 mM of aromatic donor (1-9) in Tris-HCl buffer (50 mM, pH 8.5) with a final volume of 100 μl. After one hour at 25° C., reactions were flash frozen and analyzed by HPLC. The pKa for each corresponding donor aglycon is highlighted in parentheses. (d) Plot depicting the relative Gibbs free energy of selected donors/acceptors in relation to 33a. Small glycoside donors display large shifts in relative free energy, transforming formation of UDP-Glc (33a) from an endo- to an exothermic process. The ΔGpH8.5 for 1, 2, 4, 7, and 9 with UDP in Tris-HCl buffer (50 mM, pH 8.5) at 298K relative to 33a were determined. The AG for 61a was previously determined (at pH 9.0 and 310K).

FIG. 15. The synthesis of sugar nucleotides from 2-chloro-4-nitrophenyl glucosides. (a) General reaction scheme. (b) Structures of 2-chloro-4-nitrophenyl glycoside donors evaluated for D-sugars within this series, the differences between each member and the native OleD sugar substrate (β-D-glucose) are noted. (c) Maximum observed percent conversion of (U/T)DP to (U/T)DP-glucose within a 21 hour time course assay for each donor. Standard reactions contained 7 μMTDP-16 1 mM (U/T)DP, and 1 mM of 2-chloro-4-nitrophenyl glycoside donor (9, 34-47) in Tris-HCl buffer (50 mM, pH 8.5) with a final volume of 300 μL. Over 21 hours at 25° C., aliquots taken at various times were flash frozen and analyzed by HPLC. For reactions with UDP yielding <45% conversion under standard conditions (40, 41, 43-47), identical assays using 10-fold less (U/T)DP (0.1 mM) were also conducted and, where relevant, the percent conversions for the modified reactions are represented by the darker colors. In all cases where both the α- and β-anomers were examined as donors, only the β-anomer was found to be a substrate.

FIG. 16. Evaluation of 2-chloro-4-nitrophenyl glycosides as sugar donors in coupled GT-catalyzed transglycosylation reactions. (a) The scheme for a single enzyme (TDP-16) coupled system with 4-methylumbelliferone (58) as the final acceptor (left) and a representative HPLC analysis (right) using the donor for 6-azido-6-deoxy-D-glucose (37). Reactions contained 1 mM glycoside donor, 1 mM 58, 1 mM glycoside donor in a total volume of 100 μL with Tris-HCl buffer (50 mM, pH 8.5) at 25° C. for 24 hour and were subsequently analyzed by HPLC. For the representative reaction: (i) control reaction lacking TDP-16; (ii) control reaction lacking UDP; (iii) full reaction where 37 is donor, 58 is acceptor, 59d is desired product and ⋄ represents 2-chloro-4-nitrophenolate. (b) The scheme for a double enzyme (TDP-16 and GtfE) coupled system with vancomycin aglycon (60) as the final acceptor (left) and a representative HPLC analysis (right) using the donor for 6-azido-6-deoxy-D-glucose (37). Reactions contained 1 mM glycoside donor, 0.1 mM 60, 1 mM UDP, 11 μM TDP-16, and 11 μM GtfE in a total volume of 100 μL with Tris-HCl buffer. (50 mM, pH 8.5) at 25° C. for 24 hour and were subsequently analyzed by HPLC. For the representative reaction: (i) control reaction lacking TDP-16; (ii) control reaction lacking GtfE; (iii) full reaction where 37 is donor, 60 is acceptor, 61e is desired product and ⋄ represents 2-chloro-4-nitrophenolate.

FIG. 17 (a) Scheme for colorimetric screen using the single enzyme (TDP-16) coupled format. (b) Evaluation of the colorimetric assay with 58 as the final acceptor. The reactions contained 0.5 mM 9 as donor, 0.5 mM 58 as acceptor, 5 μM UDP, and 11₃ M TDP-16 in a final total volume of 100 μl with Tris-HCl buffer (50 mM, pH 8.5) in a 96-well plate incubated at 25° C. for one hour. (i) Qualitative color change after one hour for the full reaction (square), a control lacking the final acceptor 58 (circle), and a control lacking UDP (triangle). (ii) Δ410 nm over one hour for the full reaction (squares), a control lacking the final acceptor 58 (circles), and a control reaction lacking UDP (triangles). (iii) HPLC chromatograms of full reaction at 1, 5, and 60 min where 1 is desired product, 9 is the donor, 58 is the target aglycon and ⋄ represents 2-chloro-4-nitrophenolate. (c) The absorbance data and HPLC chromatograms of three representative hits [(i) 62 (genistein), (ii) 79 (tyrphostin), or (iii) 92 (ciprofloxacin)] from the broad 50 compound panel screen using the single enzyme (TDP-16) coupled format. In HPLC chromatograms 9 indicates donor; 62, 79 or 92 represent target aglycon; ⋄ indicates 2-chloro-4-nitrophenolate; and depicts glucosylated product(s).

### DETAILED DESCRIPTION OF THE INVENTION

### In General.

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### The Invention.

The present invention provides materials and methods for exploiting glycosyltransferase reversibility for nucleotide diphosphate (NDP) sugar synthesis. The present invention provides engineered glycosyltransferase enzymes characterized by improved reaction reversibility and expanded sugar donor specificity as compared to corresponding non-mutated glycosyltransferase enzymes. Such reagents provide advantageous routes to NDP sugars for subsequent use in a variety of biomedical applications, including enzymatic and chemo-enzymatic glycorandomization.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

A “host cell” is a cell which has been transformed or transfected, or is capable of transformation or transfection by an exogenous polynucleotide sequence. A host cell that has been transformed or transfected may be more specifically referred to as a “recombinant host cell”. Preferred host cells for use in methods of the invention include bacterial cells, particularly E. coli.

The polypeptide sequence for the wild type OleD protein is provided below as SEQ ID NO: 1:MTTQTTPAHIAMFSIAAHGHVNPSLEVIRELVARGHRVTYAIPPVFADKVAATGARPVLYHSTLPGPDADPEAWGST LLDNVEPFLNDAIQALPQLADAYADDIPDLVLHDITSYPARVLARRWGVPAVSLSPNLVAWKGYEEEVAEPMWREPRQ TERGRAYYARFEAWLKENGITEHPDTFASHPPRSLVLIPKALQPHADRVDEDVYTFVGACQGDRAEEGGWQRPAGAE KVVLVSLGSAFTKQPAFYRECVRAFGNLPGWHLVLQIGRKVTPAELGELPDNVEVHDWVPQLAILRQADLFVTHAGAG GSQEGLATATPMIAVPQAVDQFGNADMLQGLGVARKLATEEATADLLRETALALVDDPEVARRLRRIQAEMAQEGG TRRAADLIEAELPARHERQEPVGDRPNGG (SEQ ID NO: 1).

An exemplary nucleotide sequence which encodes the wild type OleD protein is set forth below as SEQ ID NO: 2: atgaccaccc agaccactcc cgcccacatc gccatgttct ccatcgccgc ccacggccatgtgaacccca gcctggaggt gatccgtgaa ctcgtcgccc gcggccaccg ggtcacgtacgccattccgc ccgtcttcgc cgacaaggtg gccgccaccg gcgcccggcc cgtcctctaccactccaccc tgcccggccc cgacgccgac ccggaggcat ggggaagcac cctgctggacaacgtcgaac cgttcctgaa cgacgcgatc caggcgctcc cgcagctcgc cgatgcctacgccgacgaca tccccgatct cgtcctgcac gacatcacct cctacccggc ccgcgtcctggcccgccgct ggggcgtccc ggcggtctcc ctctccccga acctcgtcgc ctggaagggttacgaggagg aggtcgccga gccgatgtgg cgcgaacccc ggcagaccga gcgcggacgggcctactacg cccggttcga ggcatggctg aaggagaacg ggatcaccga gcacccggacacgttcgcca gtcatccgcc gcgctccctg gtgctcatcc cgaaggcgct ccagccgcacgccgaccggg tggacgaaga cgtgtacacc ttcgtcggcg cctgccaggg agaccgcgccgaggaaggcg gctggcagcg gcccgccggc gcggagaagg tcgtcctggt gtcgctcggctcggcgttca ccaagcagcc cgccttctac cgggagtgcg tgcgcgcctt cgggaacctgcccggctggc acctcgtcct ccagatcggc cggaaggtga cccccgccga actgggggagctgccggaca acgtggaggt gcacgactgg gtgccgcagc tcgcgatcct gcgccaggccgatctgttcg tcacccacgc gggcgccggc ggcagccagg aggggctggc caccgcgacgcccatgatcg ccgtaccgca ggccgtcgac cagttcggca acgccgacat gctccaagggctcggcgtcg cccggaagct ggcgaccgag gaggccaccg ccgacctgct ccgcgagaccgccctcgctc tggtggacga cccggaggtc gcgcgccggc tccggcggat ccaggcggagatggcccagg agggcggcac ccggcgggcg gccgacctca tcgaggccga actgcccgcgcgccacgagc ggcaggagcc ggtgggcgac cgacccaacg gtgggtga (SEQ ID NO: 2).

A polypeptide “substantially identical” to a comparative polypeptide varies from the comparative polypeptide, but has at least 80%, preferably at least 85%, more preferably at least 90%, and yet more preferably at least 95% sequence identity at the amino acid level over the complete amino acid sequence, and, in addition, it possesses the ability to improve conversion of NDP to NDP sugars.

The term “substantial sequence homology” refers to DNA or RNA sequences that have de minimus sequence variations from, and retain substantially the same biological functions as the corresponding sequences to which comparison is made. In the present invention, it is intended that sequences having substantial sequence homology to the nucleic acid of SEQ ID NO:2 are identified by: (1) their encoded gene product possessing the ability to improve conversion of NDP to NDP sugar; and (2) their ability to hybridize to the sequence of SEQ ID NO: 2, respectively, under stringent conditions.

As used herein, “hybridizes under stringent conditions” is intended to describe conditions for hybridization and washing under which nucleotide sequences that are significantly identical or homologous to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chlorine/sodium citrate (SSC), at about 65-70° C. (or hybridization in 4×SSC plus 50% formamide at about 42-50° C.) followed by one or more washes in 1×SSC, at about 65-70° C.

A preferred, non-limiting example of highly stringent hybridization conditions includes hybridization in 1×SSC, at about 65-70° C. (or hybridization in 4×SSC plus 50% formamide at about 42-50° C.) followed by one or more washes in 0.3×SSC, at about 65-70° C. A preferred, non-limiting example of highly stringent hybridization conditions includes hybridization in 4×SSC, at about 50-60° C. (or alternatively hybridization in 6×SSC plus 50% formamide at about 40-45° C.) followed by one or more washes in'2×SSC, at about 50-60° C. Ranges intermediate to the above-recited values, e.g., at 65-70° C. or at 42-50° C. are also intended to be encompassed by the present invention. SSPE (1×SSPE is 0.15 M NaCl, 10 mM NaH₂PO₄, and 1.25 mM EDTA, pH 7.4) can be substituted for SSC (1×SSPE is 0.15 M NaC and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6(log₁₀[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to the hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS) chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washed at 0.02M NaH₂PO₄, 1% SDS at 65° C., see e.g., Church and Gilbert (1984) Proc. Natl. Acad. Sci. USA 81: 1991-1995, (or alternatively 0.2×SSC, 1% SDS).

“Polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine, as described in a preceding definition.

The term “operably linked” means that the linkage (e.g., DNA segment) between the DNA segments so linked is such that the described effect of one of the linked segments on the other is capable of occurring. “Linked” shall refer to physically adjoined segments and, more broadly, to segments which are spatially contained relative to each other such that the described effect is capable of occurring (e.g., DNA segments may be present on two separate plasmids but contained within a cell such that the described effect is nonetheless achieved). Effecting operable linkages for the various purposes stated herein is well within the skill of those of ordinary skill in the art, particularly with the teaching of the instant specification.

As used herein the term “gene product” shall refer to the biochemical material, either RNA or protein, resulting from expression of a gene.

The term “heterologous” is used for any combination of DNA sequences that is not normally found intimately associated in nature (e.g., a green fluorescent protein (GFP) reporter gene operably linked to a SV40 promoter). A “heterologous gene” shall refer to a gene not naturally present in a host cell (e.g., a luciferase gene present in a retinoblastoma cell line).

As used herein, the term “homolog” refers to a gene related to a second gene by descent from a common ancestral DNA sequence. The term, homolog, may apply to the relationship between genes separated by the event of speciation (i.e., orthologs) or to the relationship between genes separated by the event of genetic duplication (i.e., paralogs). “Orthologs” are genes in different species that evolved from a common ancestral gene by speciation. Normally, orthologs retain the same function in the course of evolution. Identification of orthologs is important for reliable prediction of gene function in newly sequenced genomes. “Paralogs” are genes related by duplication within a genome. Orthologs retain the same function in the course of evolution, whereas paralogs evolve new functions, even if these are related to the original one.

The nucleotides that occur in the various nucleotide sequences appearing herein have their usual single-letter designations (A, G, T, C or U) used routinely in the art. In the present specification and claims, references to Greek letters may either be written out as alpha, beta, etc. or the corresponding Greek letter symbols (e.g., α, β, etc.) may sometimes be used.

The following abbreviations are used herein: GT, glycosyltransferase; NTP, nucleotide-5′-triphosphate; ATP, adenosine-5′-triphosphate; CTP, cytidine-5′-triphosphate; GTP, guanosine-5′-triphosphate; UTP, uridine-5′-triphosphate; dATP, 2′-deoxyadenosine-5′-triphosphate; dCTP, 2′-deoxycytidine-5′-triphosphate; dGTP, 2′-deoxyguanosine-S′-triphosphate; dTTP, 2′ deoxythymidine-5′-triphosphate; NDP-sugar, nucleotide diphosphosugar; IPTG, isopropyl-β-D-thiogalactopyranoside; and WT, wild-type.

In one embodiment, the present invention teaches that GTs catalyze readily reversible reactions, allowing sugars and aglycons to be exchanged. Thus, the reversibility of GT-catalyzed reactions is useful for the rapid synthesis of exotic nucleotide sugars, establishing in vitro GT activity in complex systems, and enhancing natural product diversity.

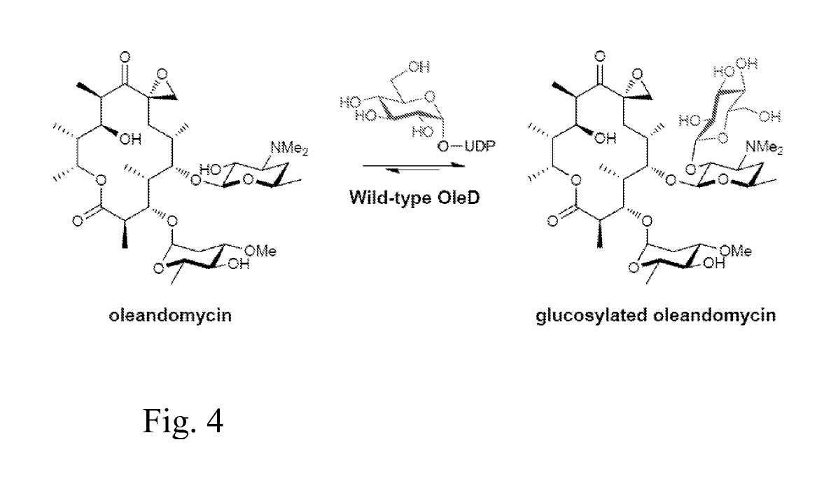

The present invention is based on the inventors' success in broadening the promiscuity of a natural product GT—the oleandomycin GT (OleD) from Streptomyces antibioticus. The native macrolide GT reaction catalyzed by OleD was previously characterized and is shown in FIG. 4.

Using a high throughput screen based upon a fluorescent glycoside donor, the inventors have identified from a small library of random OleD mutants a number of OleD variants with improved activities toward a range of alternative donor substrates. This work provides a template for engineering other natural product GTs, and highlights variant GTs for the glycorandomization of a range of therapeutically important acceptors including aminocoumarins, flavonoids and macrolides.

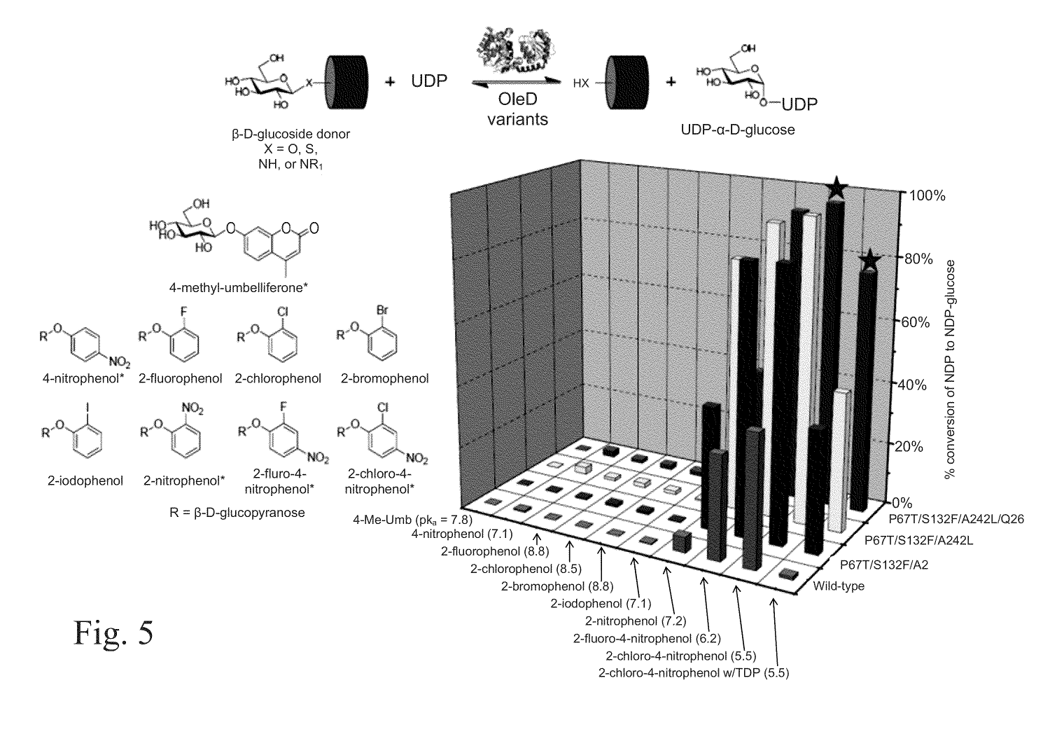

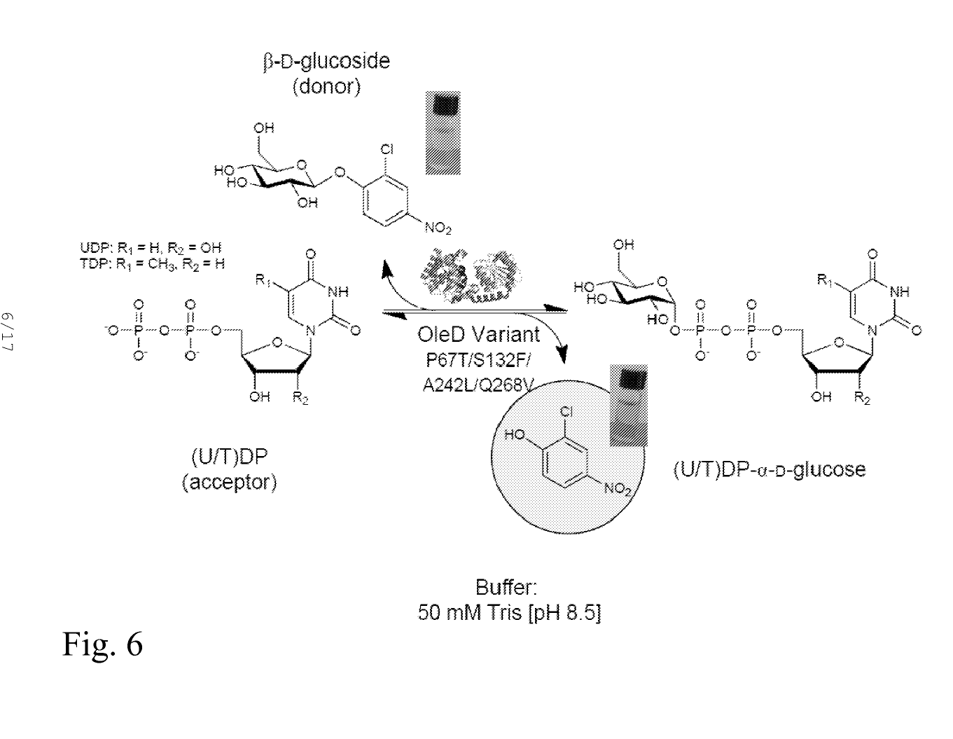

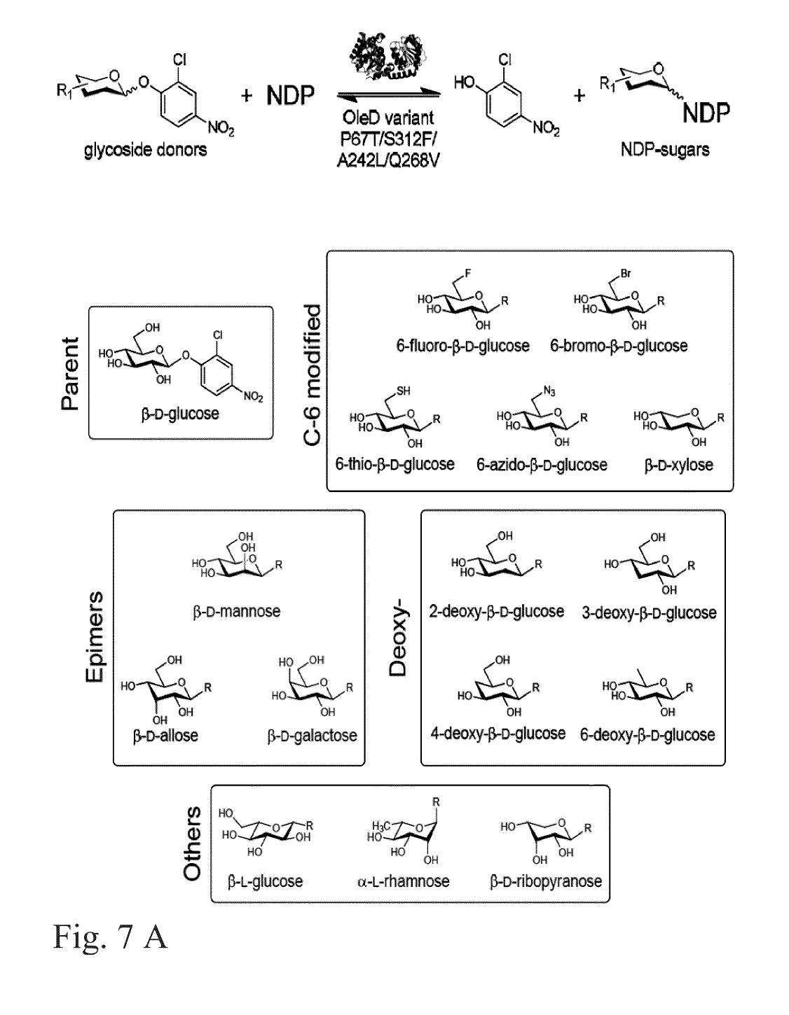

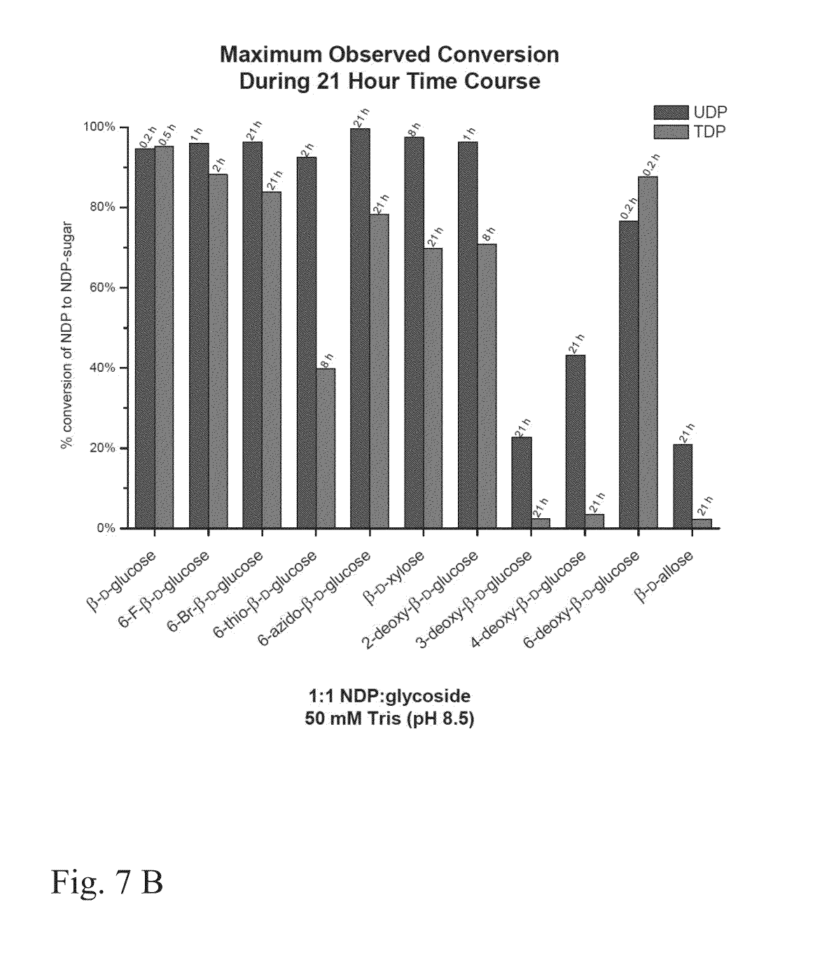

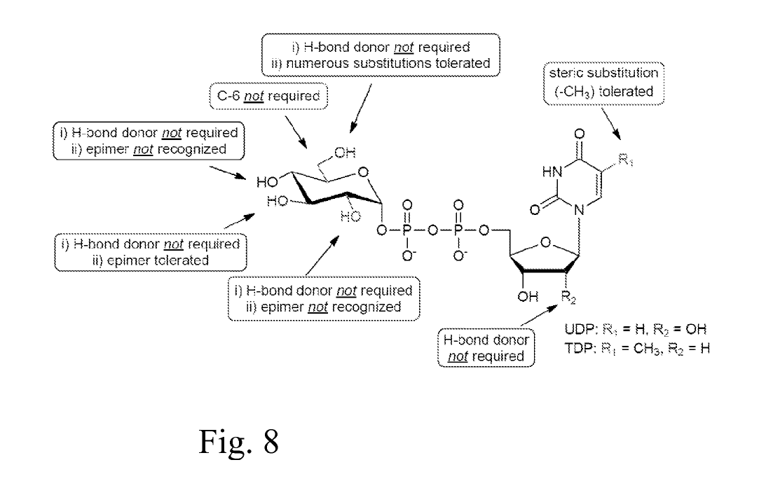

Another aspect of the invention is a versatile method for optimizing glycosyltransferases such as OleD toward non-natural donors through a comprehensive program of ‘hot spot’ saturation mutagenesis of functional positions. The method comprises a general enzyme optimization strategy (hot spot saturation mutagenesis) applicable to reactions limited by amenable high throughput screens using the macrolide glycosyltransferase OleD as a non-limiting model. Specifically, a high throughput screen based on synthetic glycoside donors is used to identify key amino acid ‘hot spots’ that contribute to GT conversion of NDP to NDP sugar. FIG. 5 illustrates the inventors' experimental strategy and results for three OleD variants assayed in combination with various synthetic glycoside donors designed to transfer a β-D-glucopyranose moiety. FIG. 6 depicts further optimization of the reaction conditions for the OleD variant P67T/S132F/A242L/Q268V. FIG. 7 illustrates conversion of NDP to NDP sugar for the same respective quadruple mutant utilizing a variety of 2-chloro-4-nitrophenol glycosyl donors, including donors for transferring exemplary sugar moieties such as β-D-glucose, C-6 modified versions of β-D-glucose, epimers of β-D-glucose, deoxy-variants of β-D-glucose, and other related sugars. Using this approach, the inventors generated 22 NDP sugars in a matter of hours. FIG. 8 depicts the inventors'conclusions regarding the structure activity relationship (SAR) between the present OleD variants and NDP sugar structures.

Accordingly, the invention provides in a first aspect an isolated mutant glycosyltransferase comprising: (a) the amino acid sequence of OleD glycosyltransferase set forth in SEQ ID NO:1, wherein proline at position 67 has been replaced with threonine, serine at position 132 has been replaced with phenylalanine, alanine at position 242 has been replaced with leucine, and glutamine at position 268 has been replaced with valine; or (b) an amino acid sequence substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine; wherein the isolated mutant exhibits an improved conversion of nucleotide diphosphate (NDP) to NDP sugar as compared to a corresponding non-mutated glycosyltransferase. In preferred embodiments, the isolated mutant glycosyltransferase is encoded by a nucleotide that hybridizes under stringent conditions to the nucleotide sequence set forth in SEQ ID NO:2.

In a second aspect, the invention provides a method of providing an isolated mutant glycosyltransferase with improved conversion of nucleotide diphosphate (NDP) to NDP sugar as compared to a corresponding non-mutated glycosyltransferase. Such a method includes steps of: (a) mutating an isolated nucleic acid sequence encoding an amino acid sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine; (b) expressing said isolated nucleic acid in a host cell; and (c) isolating from the host cell a mutant glycosyltransferase that is characterized by improved conversion of nucleotide diphosphate (NDP) to NDP sugar as compared to a corresponding non-mutated glycosyltransferase.

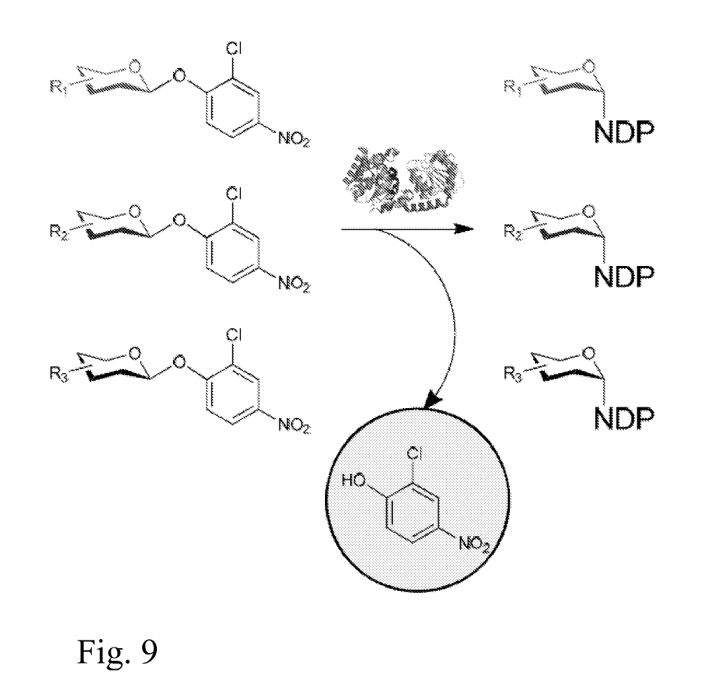

In another aspect, the invention encompasses a method of providing a nucleotide diphosphate (NDP) sugar. Such a method includes steps of incubating a nucleotide diphosphate and a glycoside donor in the presence of an isolated mutant glycosyltransferase described and claimed herein to provide an NDP sugar. FIG. 9 illustrates a general schematic for such a method using one of the synthetic glycoside donors explicitly described below.

In certain methods, the glycoside donor has the structure:

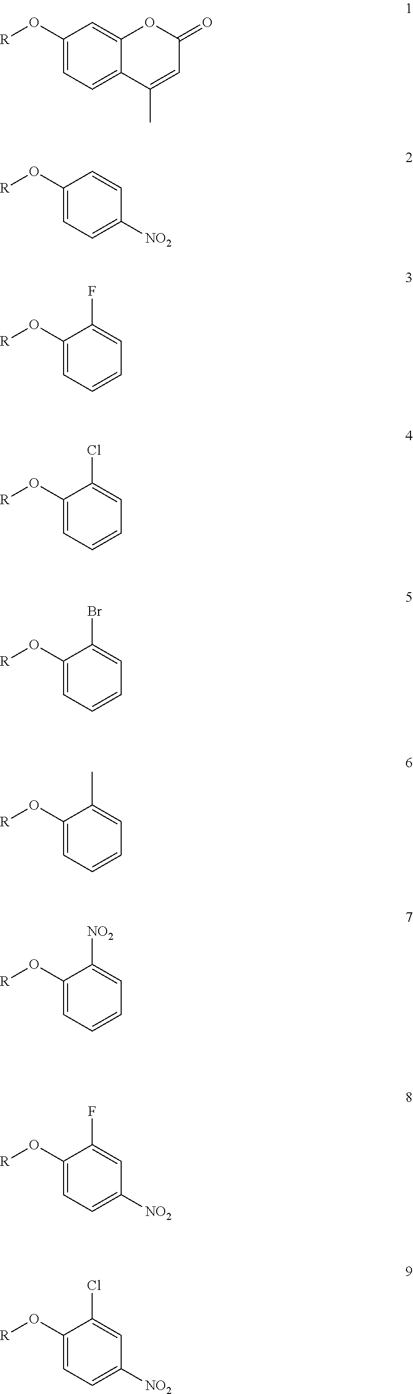

wherein R is β-D-glucopyranose.

In other embodiments, the glycoside donor has the structure:

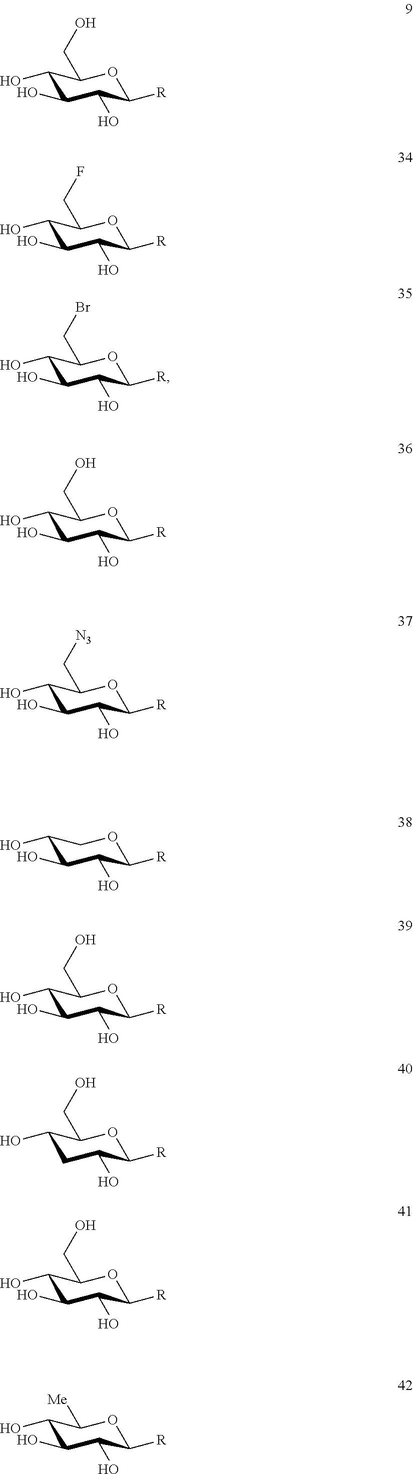

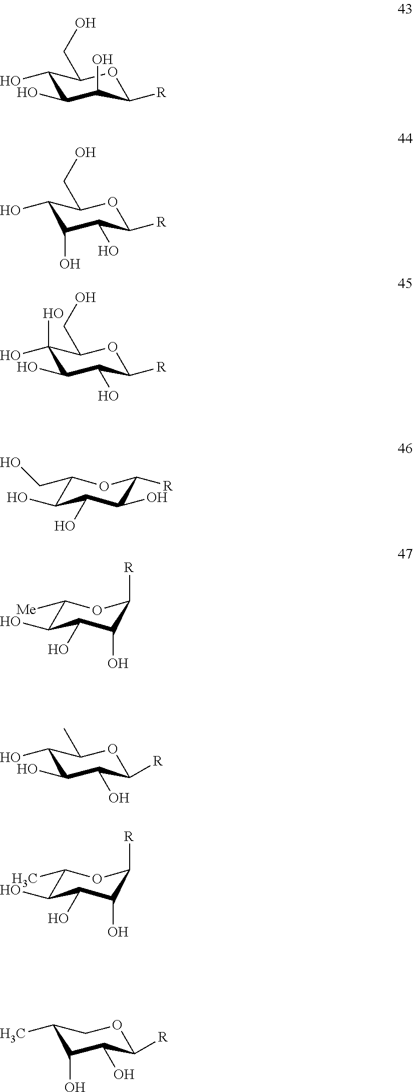

wherein R is:

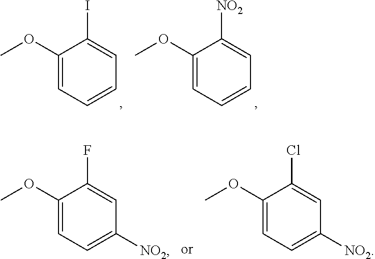

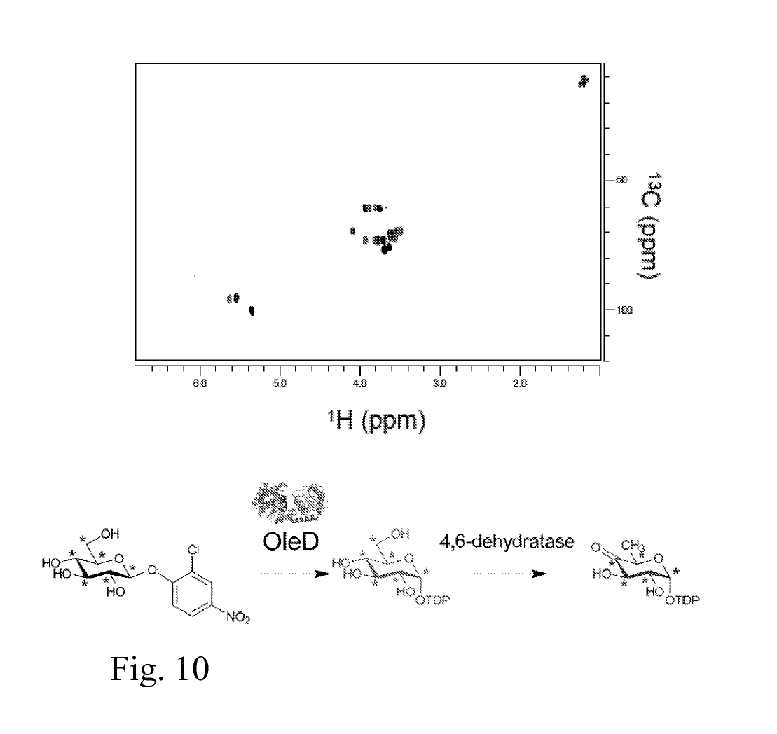

The NDP is preferably uridine or thymidine diphosphate. In alternative embodiments, the NDP sugar includes a ¹³C atom. Such labeled compounds are particularly useful for bioimaging studes, specifically nuclear magnetic resonance (NMR) imaging. FIG. 10 depicts an NMR study utilizing a ¹³C-labeled NDP sugar.

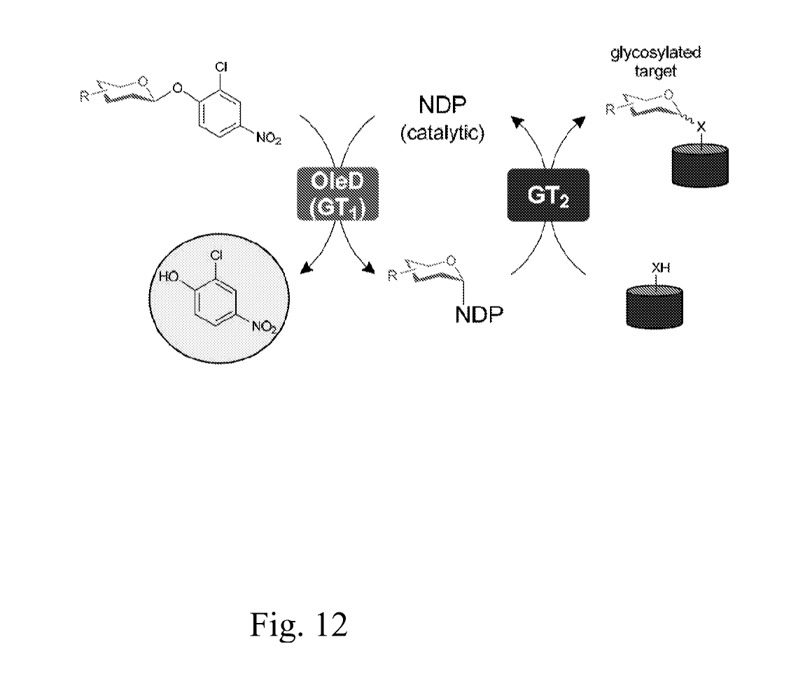

Yet another aspect of the invention is directed to a method of providing a glycosylated target molecule. Such a method includes steps of: (a) incubating a nucleotide diphosphate and a glycoside donor in the presence of an isolated mutant glycosyltransferase as described and claimed herein to provide a nucleotide diphosphate (NDP) sugar; and (b) further incubating the NDP sugar with a second glycosyltransferase and a target molecule to provide a glycosylated target molecule. FIG. 12 depicts a general scheme in which an OleD variant according to the invention is coupled with a second different GT to provide a glycosylated target molecule.

In certain embodiments, the glycoside donor has the structure:

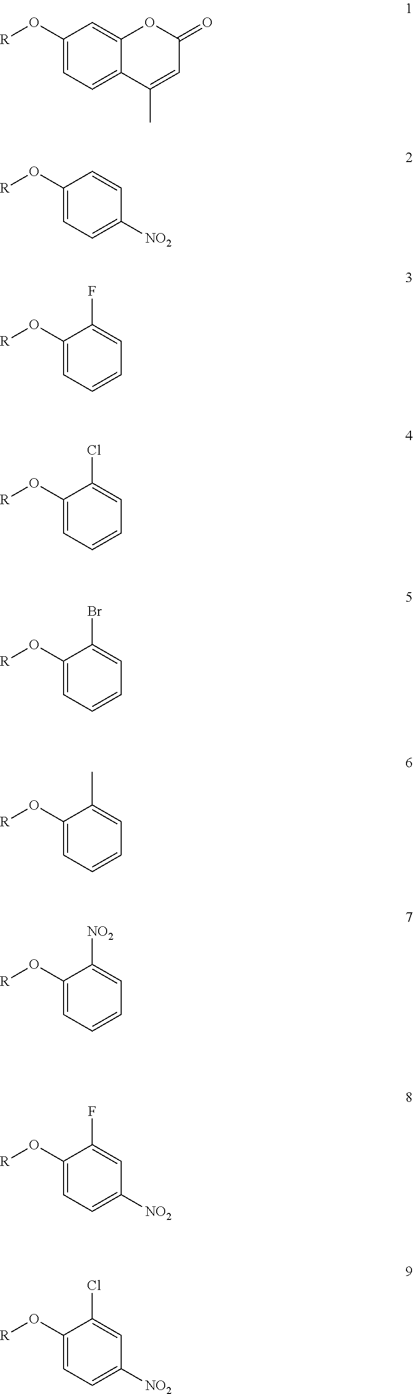

wherein R is β-D-glucopyranose.

In other embodiments, the glycoside donor has the structure:

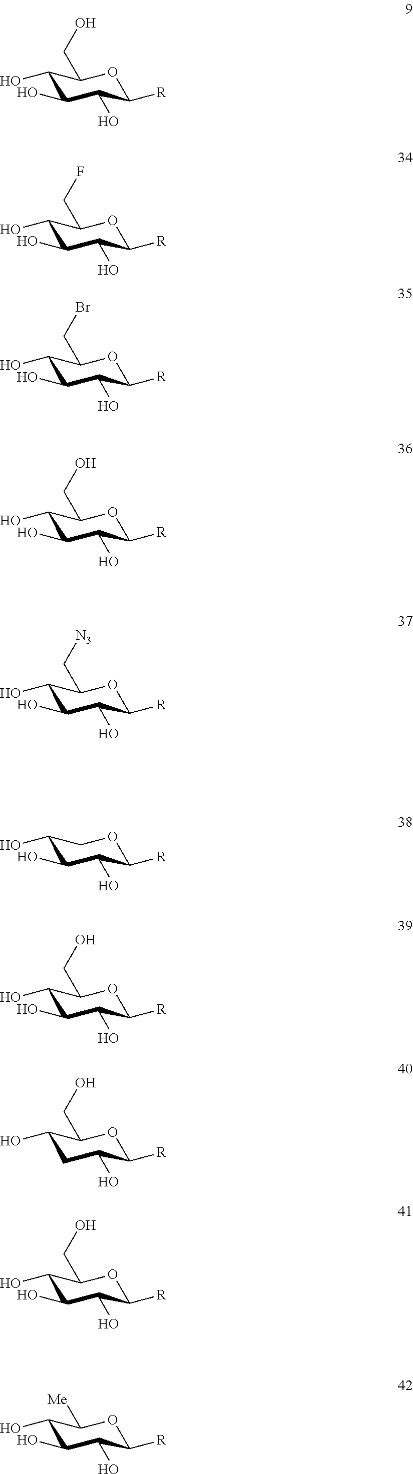

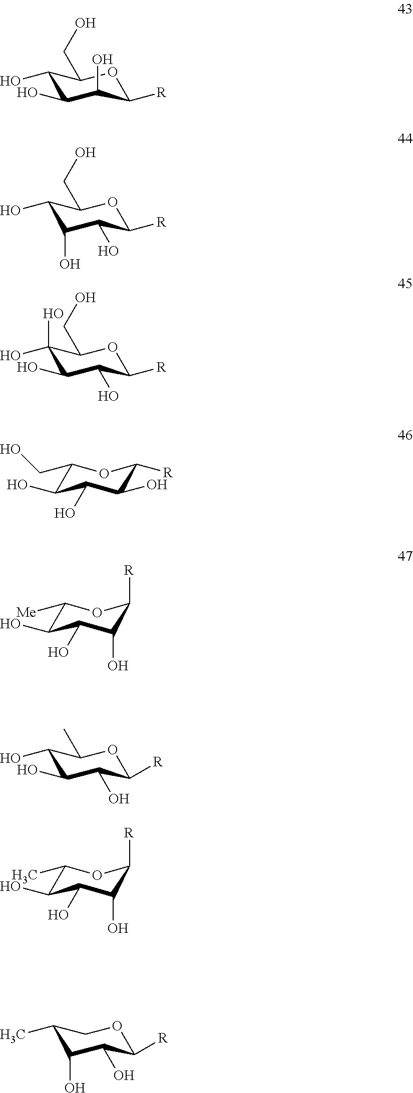

wherein R is:

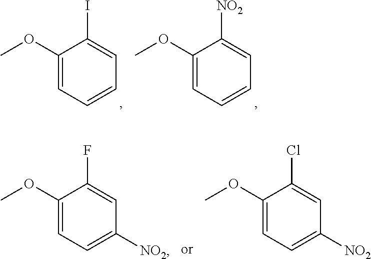

Suitable target molecules for use in the present method include, but are not limited to, natural or synthetic pyran rings, furan rings, enediynes, anthracyclines, angucyclines, aureolic acids, orthosomycins, macrolides, aminoglycosides, non-ribosomal peptides, polyenes, steroids, lipids, indolocarbazoles, bleomycins, amicetins, benzoisochromanequinones, flavonoids, isoflavones, coumarins, aminocoumarins, coumarin acids, polyketides, pluramycins, aminoglycosides, oligosaccharides, nucleosides, peptides and proteins.

In alternative embodiment, the method is carried out in vitro, preferably in a single reaction vessel.

In other embodiments, more than one type of target molecule is incubated with the second glycosyltransferase to produce a diverse population of glycosylated target molecules. As well, more than one type of NDP may be incubated with the isolated mutant glycosyltransferase to produce a diverse population of NDP sugars.

The invention further provides an isolated nucleic acid encoding a mutant glycosyltransferase having a polypeptide sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine, wherein the isolated mutant glycosyltransferase exhibits an improved conversion of nucleotide diphosphate (NDP) to NDP sugar as compared to a corresponding non-mutated glycosyltransferase.

In a preferred embodiment, the isolated nucleic acid hybridizes under stringent conditions to the nucleotide sequence set forth in SEQ ID NO:2.

In various related aspects, a recombinant vector comprising the isolated nucleic acid and a host cell comprising same are provided by the invention.

Another aspect of the invention is a fluorescent-based assay for identifying a mutant glycosyltransferase exhibiting an improved conversion of nucleotide diphosphate (NDP) to NDP sugar as compared to a corresponding non-mutated glycosyltransferase. Such a method includes steps of: (a) providing a mutant glycosyltransferase; (b) incubating the mutant glycosyltransferase with an NDP and a fluorescent glycoside donor; and (c) measuring a change in fluorescence intensity of the fluorescent glycoside donor incubated with the mutant glyscosyltransferase, the mutant glycosyltransferase's ability to transfer a sugar from said fluorescent glycoside donor to the NDP to form an NDP sugar indicated by an increase in the fluorescence of the fluorescent glycoside donor incubated with the mutant glycosyltransferase; wherein the mutant glycosyltransferase exhibits an improved conversion of NDP to NDP sugar by displaying an increase in the fluorescent glycoside donor fluorescence as compared to a corresponding non-mutated glycosyltransferase.

In certain embodiments, the glycoside donor has the structure:

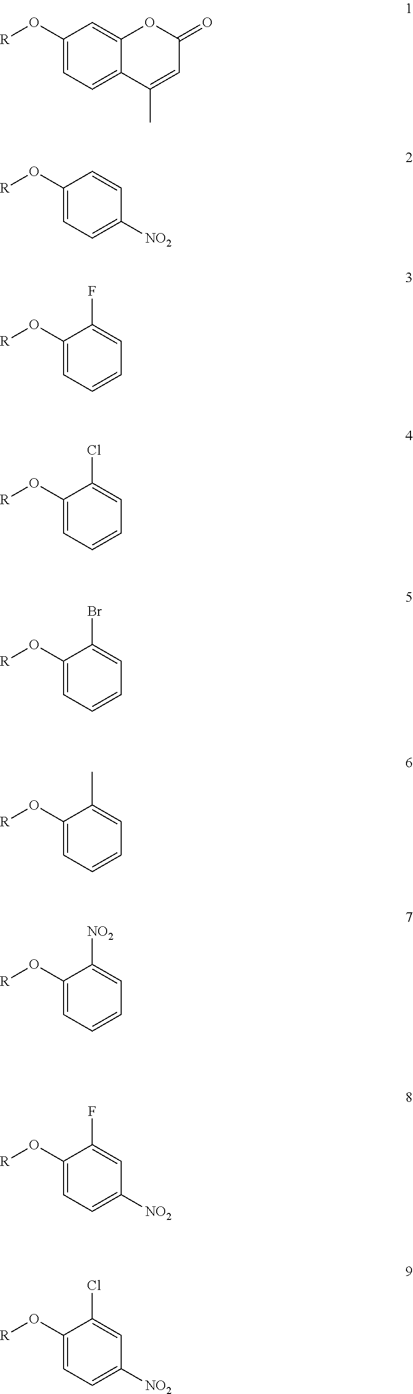

wherein R is β-D-glucopyranose.

In other embodiments, the glycoside donor has the structure:

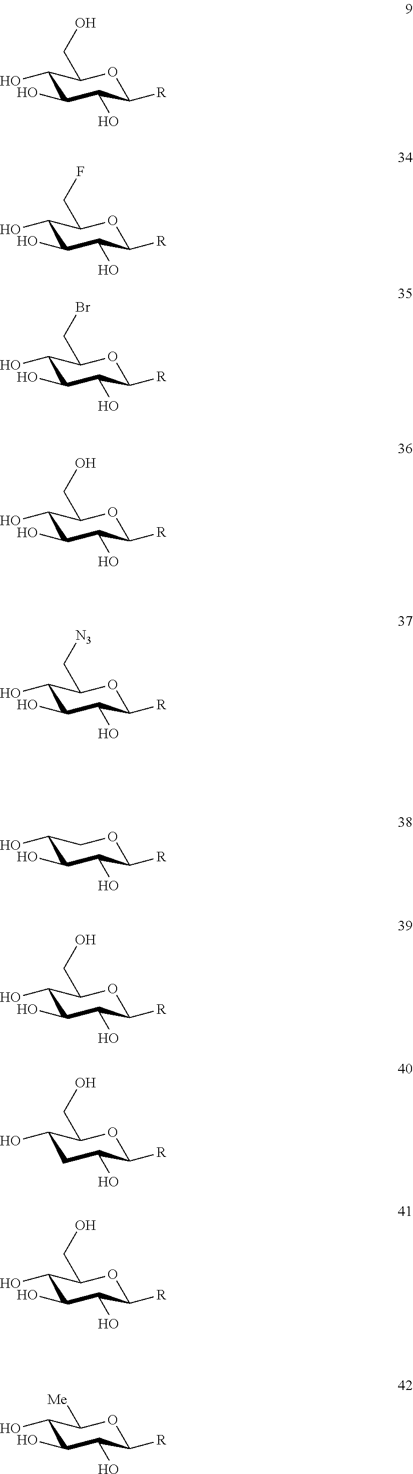

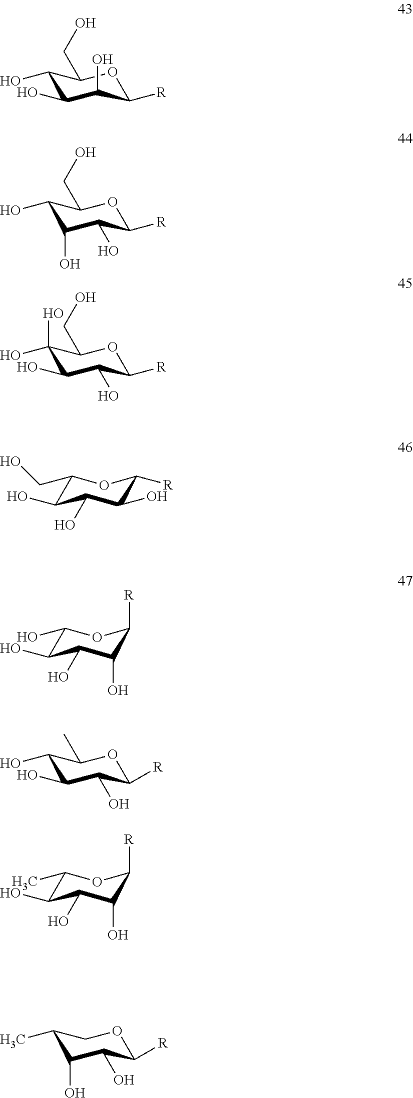

wherein R is:

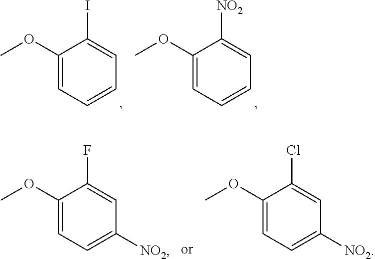

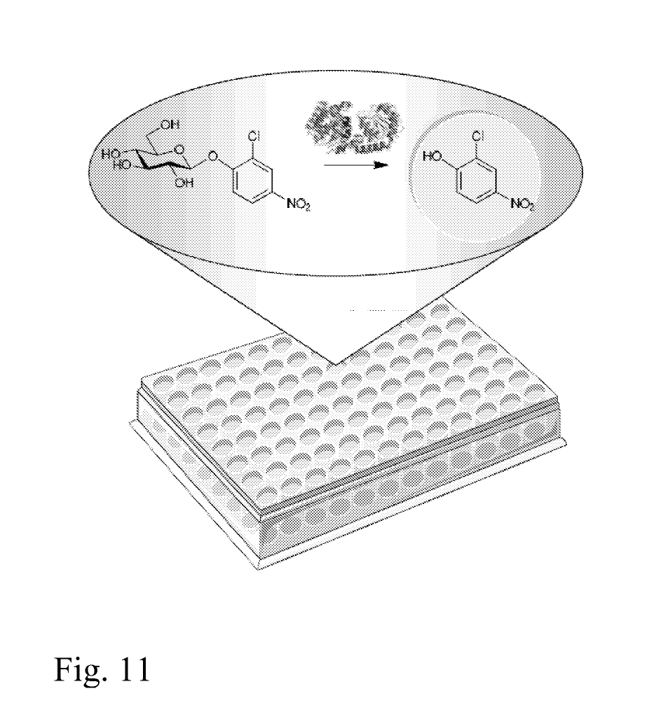

In preferred embodiments, the assay is carried out in parallel on a plurality of mutant glycosyltransferases. FIG. 11 illustrates an assay according to the invention carried out in a multiwall format.

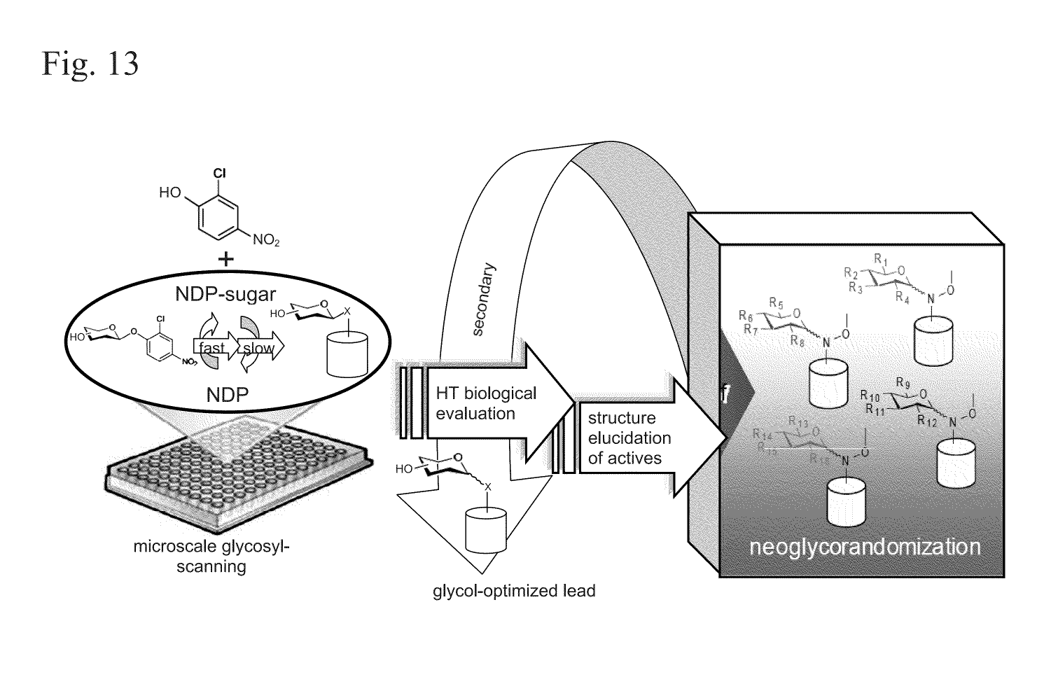

As can be appreciated, the invention provides a systematic strategy for the development of integrated high throughput pipelines to rapidly synthesize and evaluate sugar-drug conjugates—referred to as microscale glycosyl-scanning (shown generally in FIG. 13). The core innovation of this study is an unparalleled one-step glycosyltransferase (GT)-catalyzed transglycosylation reaction from simple activated (2-chloro-4-nitrophenol) glycosyl donors that ultimately drives subsequent target scaffold glycosylation while providing a convenient colorimetric readout amenable to high throughput screening. The development of this strategy utilized a novel evolved GT, a highly proficient GT for TTP/TDP, and one that lacks unwanted ‘hydrolytic’ activity) and the screening of wide array of potential donor glycosides (focusing upon those capable of presenting a fluorescent or colorimetric signal upon sugar nucleotide formation). Based upon this initial analysis, 2-chloro-4-nitrophenol glycosyl donors were found to heavily favor sugar nucleotide formation and the utility of this GT-catalyzed reaction subsequently was demonstrated with 11 diverse donors using both UDP and TDP. This study clear sets the stage for microscale scanning (toward either diverse drug and/or sugar scaffolds).

Glycosyltransferases (GTs) constitute a predominant enzyme superfamily responsible for the attachment of carbohydrate moieties to a wide array of acceptors that include nucleic acids, polysaccharides, proteins, lipids, carbohydrates and medicinally relevant secondary metabolites (1, 2). The majority of GTs are LeLoir (sugar nucleotide-dependent) enzymes and utilize nucleoside diphosphate sugars (NDP-sugars) as ‘activated’ donors for glycosidic bond formation. Recent studies have revealed certain GT-catalyzed reactions from bacterial secondary metabolism to be reversible, presenting new GT-catalyzed methods for NDP-sugar synthesis as well as the GT-catalyzed exchange of sugars attached to complex natural products including glycopeptides, enediynes (3), macrolides (4), macrolactams (5), (iso)flavonoids (6) and polyenes (7). Yet, consistent with the general perception of NDP-sugars as ‘activated’ sugar donors, the thermodynamics of reactants/products in such reactions heavily disfavor NDP-sugar formation (i.e., with NDP-sugar as product, Keq<1) (3, 8, 9), severely restricting the synthetic utility of GT-catalyzed reactions run in reverse.

To address these limitations, herein we report the incorporation of simple aromatic glycosides in such reactions dramatically alters the equilibrium of GT-catalyzed reactions and thereby enables a variety of novel transformations including: i) the syntheses of NDP-sugars, ii) a coupled GT-catalyzed platform for the differential glycosylation of small molecules (including natural products and synthetic drugs/targets), and iii) a colorimetric readout upon glycosyltransfer amenable to high throughput formats for glycodiversification and glycoengineering which can be coupled to nearly any downstream sugar nucleotide-utilizing enzyme.

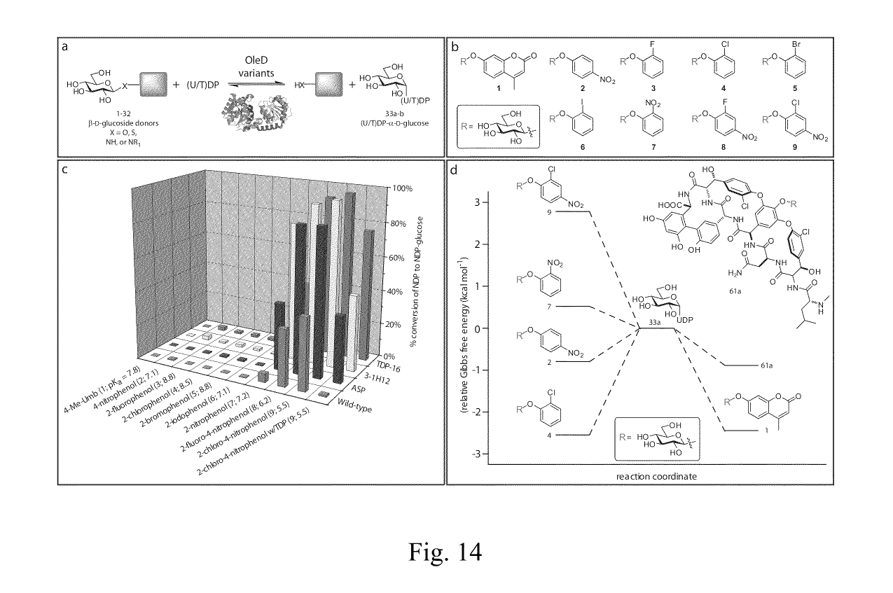

The availability of a GT capable of utilizing a wide array of both simple aromatic acceptors and sugar nucleotides set the stage for this systematic study. With the aid of a crystal structure (10), previous directed evolution and engineering of an inverting macrolide-inactivating GT from S. antibioticus (OleD) identified several highly permissive variants for both sugar nucleotides (14 known sugar substrates) and acceptors (>70 structurally diverse known substrates) in the context of the forward reaction (11-16). Utilizing the aglycons recognized in forward reactions as a template, a set of 32 putative ü-D-glucopyranosides donors were synthesized and tested against a series of OleD variants in reverse reactions for production of UDP-J-D-glucose (UDP-Glc, 33a) in the presence of UDP (FIG. 14a). The syntheses of these putative donors required 1-3 steps (37% average overall yield) and, in all but one case (19), provided the desired E-anomer exclusively. Of the 32 putative donors evaluated, 9 (1-9) led to UDP-Glc (33a) formation with all variants examined (FIG. 14b). This systematic analysis revealed a clear correlation between the leaving group ability of the sugar donor and the production of desired sugar nucleotide wherein the combination of OleD variant TDP-16 (containing the mutations P67T/S132F/A242L/Q268V) (15) and 2-chloro-4-nitrophenyl ü-D-glucopyranoside (9) provided the best overall yields of UDP-Glc (33a) or TDP-Glc (33b) (FIG. 14c).

Using this preferred donor, maximal turnover was observed at pH 7.0-8.5, a range consistent with the previously reported pH-rate profile for the wt OleD in the forward direction (8). NDP-sugar formation was also observed in the presence of ADP and GDP, albeit with much lower efficiency than with UDP or TDP. Thus, four of the five standard nucleotide moieties utilized by all LeLoir GTs (including) not only natural product GTs but also those which catalyze the formation of glycoproteins (17-19), oligosaccharides (19-23), glycolipids (24), glycoconjugates (1), etc.) are accessible via this method. To demonstrate preparative scale and provide material for full characterization, this reaction was conducted with a 1:1 molar ratio of glucoside donor to NDP using 9 mg of UDP or TDP to provide 6.9 (55% isolated yield) and 7.7 mg (61% isolated yield), of UDP-Glc (33a) and TDP-Glc (33b).

Under saturating donor 9, kinetic analysis revealed the kcat/Km of TDP-16 to be improved by a factor of 25 or 315 (varied UDP or TDP, respectively) compared to wild-type OleD, consistent with this mutant's enhanced proficiency toward TDP-sugars (15). Equilibrium constants (Keq, pH8.5) were also determined for 1, 2, 4, 7, and 9 and utilized to calculate the corresponding Gibbs free energy according to equation (1).

ΔG°_{pH8.5}=−RT ln(_{Keq,pH8.5})

In agreement with the typically observed thermodynamics for these reactions (3, 8, 9), the 4-, 1-, or 2-(ΔG°_{pH8.5}=+2.55, +2.44, and +0.92 kcal mol-1, respectively) UDP-Glc transformations were endothermic. In stark contrast, 7- or 9-(ΔG°_{pH8.5}=−0.52 and −2.78 kcal mol-1, respectively) UDP-Glc transformations were notably exothermic (FIG. 14d) and thereby correspond to a dramatic shift of GT-catalyzed reaction Keq which markedly favors NDP-sugar formation.

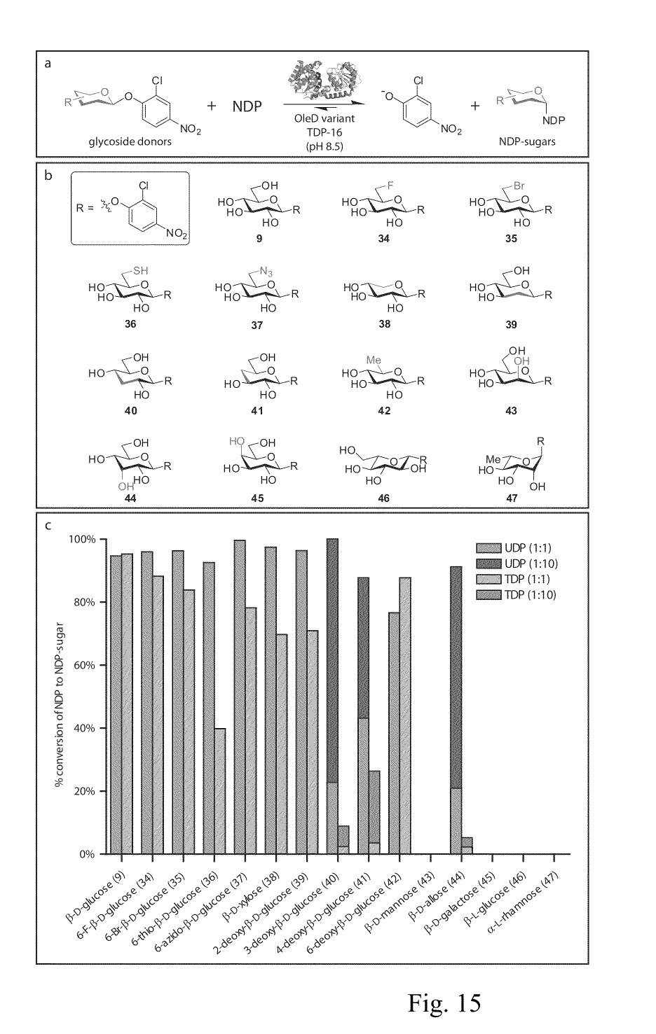

To further assess the utility of this reaction toward novel sugar nucleotide synthesis, 15 additional 2-chloro-4-nitrophenyl glycosides (34-47) were synthesized and evaluated for production of the corresponding sugar nucleotides in presence of UDP and TDP (FIG. 15a-b). This set of putative donors represents a series of uniquely functionalized gluco-configured sugars as well as corresponding epimers (C2, C3, C4), deoxy (C2, C3, C4 and C6) analogues and even L-sugars. The syntheses of these putative donors required 2-7 steps (35% average overall yield) and, in all cases, exclusively provided the desired anomeric stereochemistry.

Of the 15 glycoside donors evaluated (9, 34-47) with TDP-16, 11 (9, 34-42, 44) resulted in the formation of the desired sugar nucleotide with both UDP and TDP (FIG. 15c). With a 1:1 molar ratio of (U/T)DP to glycoside donor in these 11 reactions, an average conversion of 66% was observed, once again highlighting the thermodynamic driving force provided by the aromatic sugar donor. For a small subset of donors (40, 41, 44), a shift of the ratio to 1:10 of UDP to glycoside donor drastically increased yields of UDP-sugars (54a, 55a, 57a) to >85%, while yields of TDP-sugars (54b, 55b, 57b) remained low (<25%) (FIG. 15c).

In notable contrast to the prior use of GT-catalyzed reactions for the synthesis of single sugar nucleotides (wherein a molar ratio of up to 1:100 sugar donor to NDP was required and, in all cases, <50% desired sugar nucleotide product was observed) (3-5, 7, 9), this study reveals a truly useful synthetic transformation (wherein a 1:1 molar ratio of sugar donor to UDP provides >70% yield desired sugar nucleotide product for 8 out of 11 examples examined). In the context of sugar nucleotide synthesis, this GT-catalyzed method offers a noteworthy alternative to both conventional chemoenzymatic approaches (requiring 1 to 11 enzymes with typical overall yields from unprotected sugars ranging from 10% to 35%) (25-30) and multi-step chemical syntheses (requiring 3-11 total steps for coupling sugar-1-phosphates and activated NMPs with typical overall yields from peracetylated sugars ranging from 9-66%) (31-33).

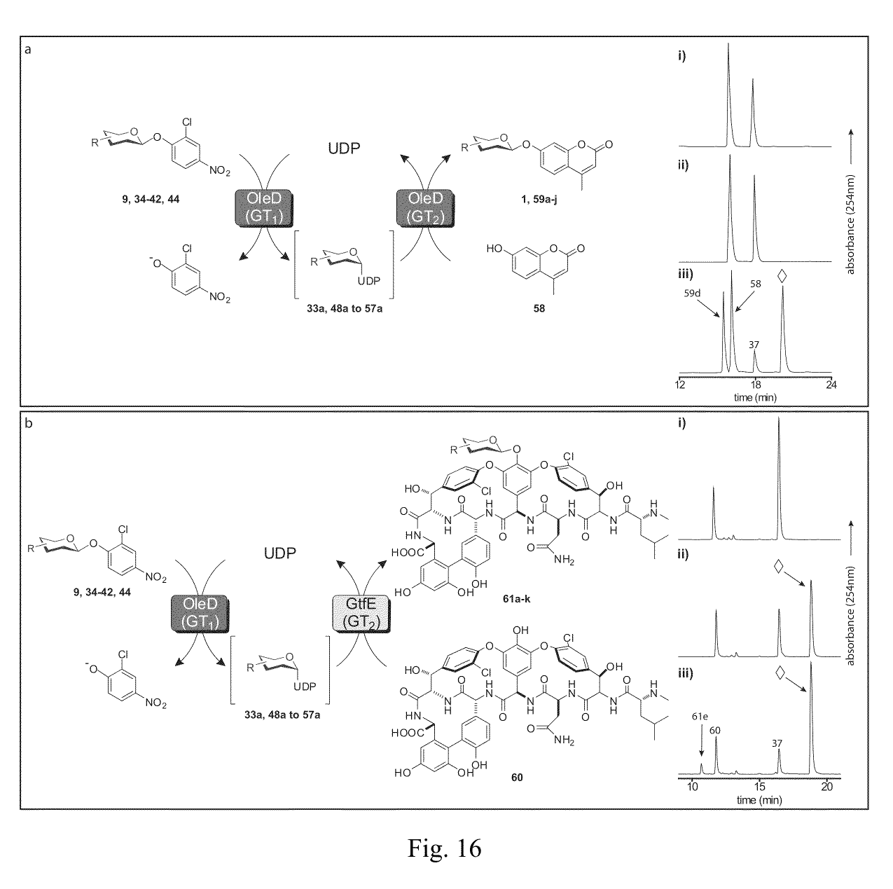

The previously reported promiscuity of OleD variants in forward reactions (11-16), coupled with the newly demonstrated ability to synthesize large numbers of NDP-sugars in situ, raised the question of whether TDP-16 could enable a one-pot transglycosylation wherein the sugar nucleotide formed from 9 (via the ‘reverse reaction’) could serve as a donor for a subsequent glycoside-forming reaction (via a simultaneous ‘forward reaction’) (FIG. 16a). Such single and double enzyme ‘aglycon exchange’ reactions have been previously reported in the context of complex natural products (3-5, 7, 9), but again, the Keq of such reactions has restricted their general utility. To assess the potential of a single GT-catalyzed transglycosylation, a series of model reactions, each containing the aglycon acceptor 4-methylumbelliferone (58; 1 mM), one member of the 2-chloro-4-nitrophenyl glycoside donor series (9, 34-42, 44; 1 mM), UDP (1 mM) and OleD variant TDP-16 (11 ₃M), revealed all 11 expected products (1, 59a-59j) with an average yield of 45%.

For comparison, the yield of 1 in the single GT coupled reaction was 62% (n=1), while the average yield of 1 via a standard OleD catalyzed forward reaction (using 1 equivalent of UDP-Glc donor) was 60%±3% (n=3). Given the established ability of OleD variants to glycosylate a wide array of structurally-diverse small molecules, drugs and natural products (11-16), the extension of this OleD-catalyzed single pot transglycosylation (or ‘aglycon exchange’) reaction is anticipated to offer a variety of opportunities for the glycodiversification of bioactive molecules including a number of clinically-approved drugs and complex natural products.

To further probe the potential of in situ NDP-sugars from synthetic donors to ultimately serve as donors for GTs other than OleD variants, a series of dual GT-catalyzed model reactions were performed. For this set of model reactions, GtfE was selected because of its known NDP-sugar promiscuity (34, 35) as well as the clinical potential of glycodiversified vancomycin analogues (36, 37). Typical reactions for this assessment contained a NDP-sugar generating component consisting of a 2-chloro-4-nitrophenyl glycoside donor (9, 34-42 or 44; 1 mM), UDP (1 mM) and OleD variant TDP-16 (11 ₃M) coupled to a glycoside-forming component with vancomycin aglycon (60; 0.1 mM) and the vancomycin aglycon glucosyltransferase GtfE (11 ₃M). Remarkably, this series of dual GT-catalyzed transglycosylation (or ‘aglycon exchange’) reactions also led to the formation of all 11 expected products (61a-61k) with an average yield of 36%. As a comparison, the yield of 61a in the dual-GT coupled reaction was 53% (n=1), while the average yield of 61a via a standard GtfE catalyzed forward reaction (using 10 equivalents of native substrate UDP-Glc) was 53%±0.5% (n=3). Thus, this convenient 2-chloro-4-nitrophenyl glycoside donor-driven coupled format presents synthetically useful novel sugar nucleotides in situ without the need for the tedious a priori sugar nucleotide synthesis and/or purification. Furthermore, the formation of the colorimetric product 2-chloro-4-nitrophenolate (Omax=398 nm; H410=2.4×104 M-1 cm-1; pH 8.5) upon GT-catalyzed glycoside formation in these single or dual GT-catalyzed coupled reactions also offers a unique opportunity for high throughput screening as described below.

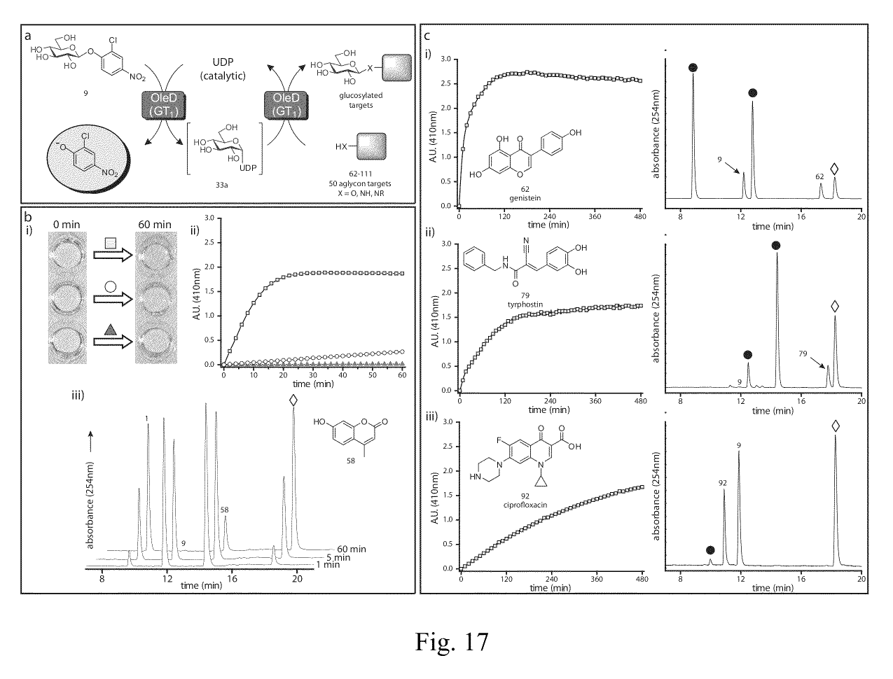

The 2-chloro-4-nitrophenolate released during the course of GT-catalyzed NDP-sugar formation directly, or in the context of the coupled reactions formats presented, can be followed spectrophotometrically at 410 nm in real-time (FIG. 17). The ability to do so presents one of the first truly general continuous GT assays as the colorimetric read-out directly correlates to NDP-sugar usage in such reactions and thereby avoids the need for additional manipulations or specialized probes commonly associated with conventional assays for glycosidic bond formation (38-40). To demonstrate this approach, a set of 50 (62-111) medicinally relevant compounds were screened with the single GT-catalyzed reaction in a high throughput format.

Specifically, each 100 PI reaction in the 96-well plate contained the sugar donor 9 (0.5 mM), a putative aglycon acceptor (0.5 mM), a catalytic amount of UDP (5 ₃M) and TDP-16 (11 ₃M) and reaction progress was monitored at 410 nm over 480 min. Notably, the use of UDP as a limiting reagent within this coupled system reduces the potential for the various types of inhibition commonly observed in forward GT-catalyzed reactions with NDP and NDP analogues (1, 8). Based upon this cumulative rapid analysis, 43 compounds (62-103) led to a positive response (designated as three standard deviations above the mean for control reactions), 37 of which (62-93, 96, 97, 99, 101, 103) were subsequently confirmed by HPLC and/or LC/MS to lead to products consistent with glucoside formation. While this example clearly illustrates the utility of this high throughput assay to identify novel acceptors that can be glycosylated by a given GT, given this demonstrated ability to couple this assay to essentially any downstream sugar-utilizing enzyme/process, this assay is also anticipated to have a broad range of fundamental applications including screens for GT inhibitors, GT engineering/evolution (toward utilization of novel NDPs, glycoside donors, and/or acceptors) (38, 39, 41-43) and/or engineering/evolution/investigations of additional NDP-sugar utilizing enzymes (17-24, 44, 45).

In summary, this study directly challenges the general notion that NDP-sugars are ‘high-energy’ sugar donors when taken out of their traditional biological context, revealing the equilibria of GT-catalyzed reactions to be highly substrate-dependent and adaptable. The flexibility of the GT thermodynamic landscape, in turn, enabled general NDP-sugar syntheses, in situ formation of NDP-sugars to drive coupled LeLoir GT-catalyzed reactions for glycoconjugate formation, and the first general high throughput colorimetric assay for glycosyltransfer. Given the power of the screen presented, these preliminary data suggest both the ability to enable the rapid optimization (via directed evolution) of new OleD prodigy for nearly any desired NDP/sugar pair as well as the ability to couple this screen to nearly any downstream sugar-utilizing for engineering/evolution or biochemical analysis. While substrates providing the greatest thermodynamic advantage may not always provide an equivalent kinetic advantage, this study also highlights the merit of optimizing enzyme-catalyzed reactions based upon thermodynamic constraints. We anticipate future attempts to exploit and/or engineer other novel enzyme-catalyzed reactions may benefit from similar considerations.

### EXAMPLES

The following experimental data are provided to illustrate the invention. It is to be understood that a person skilled in the art who is familiar with the methods may use other yeast strains, recombinant vectors, and methodology which can be equally used for the purpose of the present invention. These alterations are included in the scope of the invention.

### Example 1

Materials.

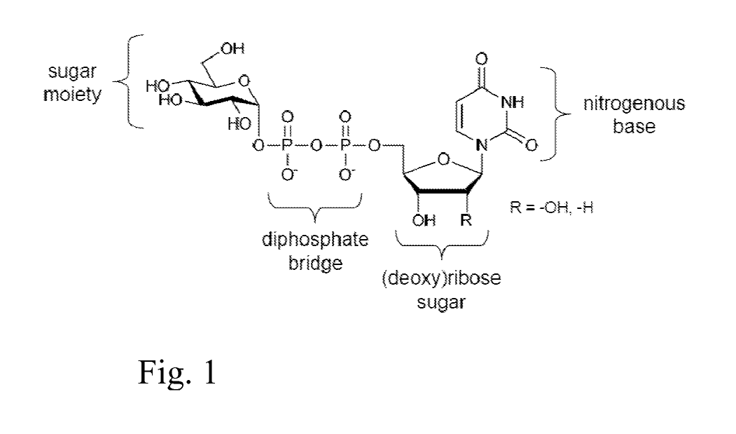

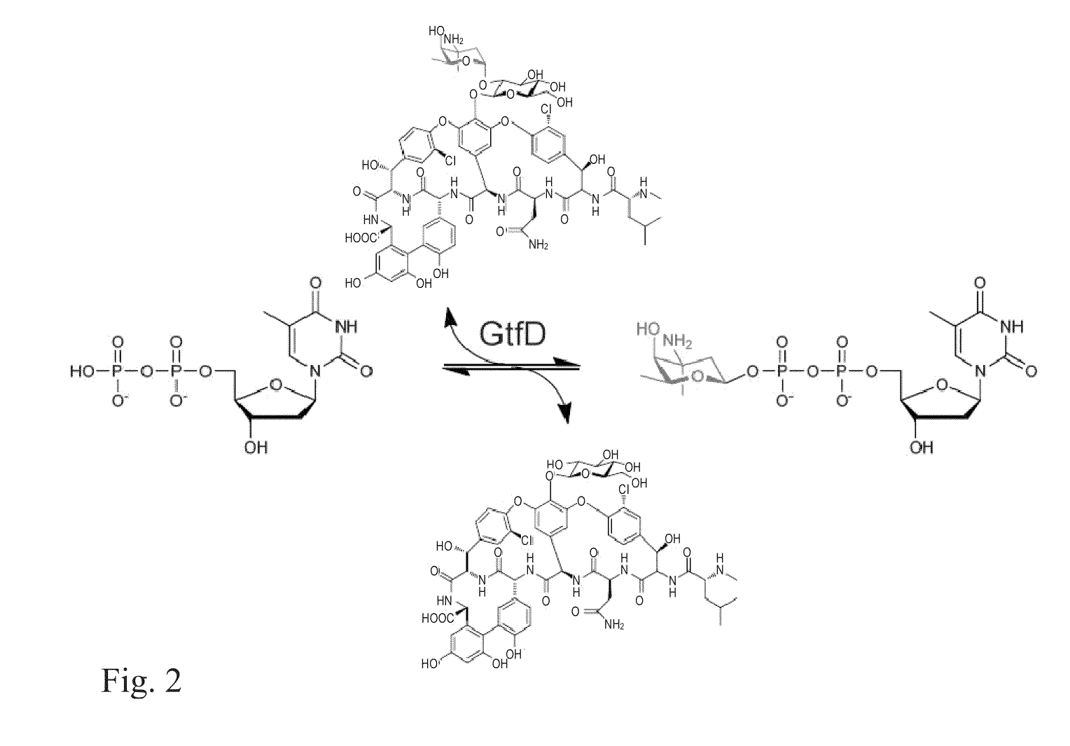

Bacterial strain E. coli BL21(DE3)pLysS was from Stratagene. NovaBlue was from Novagen. Plasmid pET28/OleD was a generous gift from Prof Hung-Wen Liu (University of Texas-Austin, Austin, USA) and pET28a was from Novagen. All other chemicals were reagent-grade purchased from Fluka, New England Biolabs, or Sigma, unless otherwise stated. Primers were ordered from Integrated DNA Technologies (Coralville, Iowa). Oleandomycin was purchased from MP Biomedicals Inc. (Ohio, USA). Phenolic substrates (Table 1: 27, 28, 30-32) were from Indofine Chemical Company Inc. (Hillsborough, N.J., USA). Novobiocic acid (Table 1: 29) was prepared as previously described from Novobiocin. Albermann C, et al. (2003) Org Lett 5: 933-6. Product standard 4-Me-umb-7-O-beta-D-glucoside (FIG. 1: 4-glc) was from Sigma, daidzein-7-O-beta-D-glucoside (Table 1: 31-glc), and genistein-7-O-beta-D-glucoside (Table 1: 32-glc) standards were from Fluka. Analytical HPLC was performed on a Rainin Dynamax SD-2/410 system connected to a Rainin Dynamax UV-DII absorbance detector.

Mass spectra were obtained using electrospray ionization on an Agilent 1100 HPLC-MSD SL quadrapole mass spectrometer connected to a UV/Vis diode array detector.

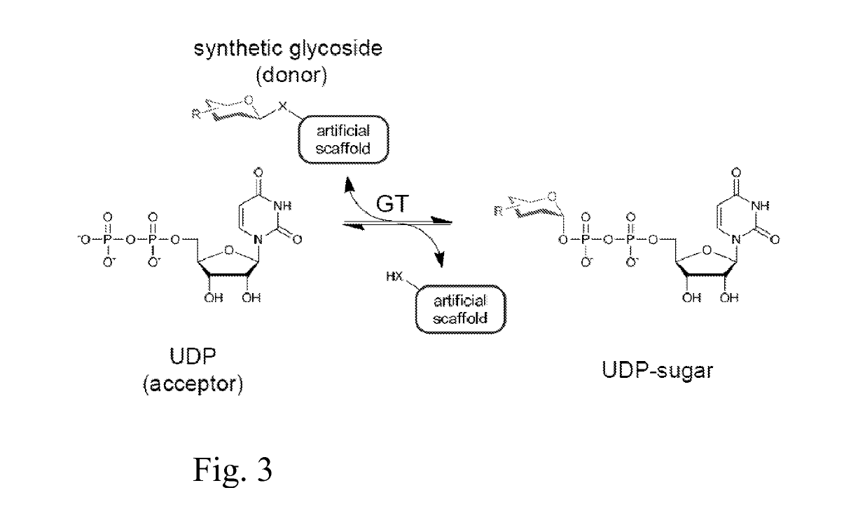

For LC-MS analysis, quenched reaction mixtures were analyzed by analytical reverse-phase HPLC with a 250 mm×4.6 mm Gemini 5 [symbol] C18 column (Phenomenex, Torrance, Calif.) using a gradient of 10-90% CH₃CN in 0.1% formic acid/H₂O in 20 min at 1 ml/min, with detection at 254 nm. The enzymatic and/or chemical syntheses sugar nucleotides (FIG. 3: 7-9, 11-25) utilized in this study have been previously described. Borisova S A, et al. (2006) Angew Chem Int Ed Engl 45: 2748-53; Barton W A, et al. (2002) Proc Natl Acad Sci USA 99: 13397-402; Fu X, et al. (2003) Nat Biotechnol 21: 1467-9; Zhang C, et al. (2006) Science 313: 1291-4; Borisova S A, et al. (2006) Angew Chem Int Ed Engl 45: 2748-53; Jiang J, et al. (2001) Angew Chem Int Ed Engl 40: 1502-1505; Losey H C, et al. (2002) Chem Biol 9: 1305-14. Donors 2, 6, and 10 (FIG. 3) were from Sigma.

Glycosyltransferase Mutant Library Preparation.

The random mutant library was prepared via error-prone PCR using the Stratagene GeneMorph II Random Mutagenesis Kit, as described by the manufacturer using varying quantities of pET28/OleD as template. The primers used for amplification of the OleD gene were T7 FOR (5′-TAA TAC GAC TCA CTA TAG GG-3′; SEQ ID NO:3) and T7 REV (5′-GCT AGT TAT TGC TCA GCG G-3′; SEQ ID NO:4). Amplified product was digested with NdeI and HindIII, purified by agarose gel electrophoresis (0.8% w/v agarose), extracted using the QIAquick Gel Extraction Kit (QIAgen, Valenica, Calif.), and ligated into similarly treated pET28a. The ligation mixtures were transformed into chemically competent NovaBlue cells and single colonies used to prepare plasmid for DNA sequencing, which revealed that a library made with ^{˜}10 ng starting template had the desired mutation rate of 1-2 amino acid mutations per gene product. Subsequently, all the transformants from this library were pooled and cultured overnight. Plasmid was prepared from this culture and used to transform chemical competent E. coli BL21(DE3)pLysS, which was screened as described below.

Site-Directed Mutagenesis.

Site-specific OleD variants were constructed using the Stratagene QuikChange II Site-Directed Mutagenesis Kit, as described by the manufacturer. The amplified plasmid was digested with DpnI and transformed into chemical competent E. coli XL1 Blue. Constructs were confirmed to carry the correct mutation(s) via DNA sequencing.

Screening. Individual colonies were used to inoculate wells of a 96-deep well microtitre plate wherein each well contained 1 ml of LB medium supplemented with 50 micrograms/ml kanamycin. Culture plates were tightly sealed with AeraSeal™ breathable film (Research Products International Corp.). After cell growth at 37° C. for 18 h with shaking at 350 rpm, 100 microliters of each culture was transferred to a fresh deep-well plate containing 1 ml of LB medium supplemented with 50 micrograms/ml kanamycin. The original plate was sealed and stored at 4° C., or a glycerol copy made by mixing 100 microliters of each culture with 100 microliters 50% (v/v) glycerol and storing at −80° C. The freshly inoculated plate was incubated at 37° C. for 2-3 h with shaking at 350 rpm. Expression of the N-terminal His 6-tagged OleD was induced at OD600 ^{˜}0.7, and isopropyl beta-D-thiogalactoside (IPTG) was added to a final concentration of 0.4 mM and the plate incubated for 18 h at 18° C. Cells were harvested by centrifugation at 3000 g for 10 min at 4° C., the cell pellets thoroughly resuspended in chilled 50 mM Tris-HCl (pH 8.0) containing 10 mg/ml lysozyme (Sigma), and the plates were subjected to a single freeze/thaw cycle to lyse the cells. Following thawing, cell debris was collected by centrifugation at 3000 g for 20 min at 4° C. and 50 microliters of the cleared supernatant used for enzyme assay.

For the assay, cleared supernatant was mixed with an equal volume (50 microliters) of 50 mM Tris-HCl (pH 8.0) containing 10 mM MgCl2, 0.2 mM 4-Me-umb (FIG. 1: 4), and 1.0 mM UPDG (FIG. 3: 2) using a Biomek FX Liquid Handling Workstation (Beckman Coulter, Fullerton, Calif.). Upon mixing, the fluorescence at excitation 350 nm and emission 460 nm was measured using a FLUOstar Optima plate reader (BMG Labtechnologies, Durham, N.C.) and the reactions incubated for 4 h at 30° C., at which time the fluorescence measurement was repeated. Activity of the clones was expressed as the difference in fluorescence intensity between 0 h and 3 h.

Protein expression and purification. For characterization of specific OleD variants, single colonies were used to inoculate 3 ml LB medium supplemented with 50 micrograms/ml kanamycin and cultured overnight at 37° C. The entire starter culture was then transferred to 1 liter LB medium supplemented with 50 micrograms/ml kanamycin and grown at 37° C. until the OD600 was ^{˜}0.7, then IPTG to a final concentration of 0.4 mM was added and the flask incubated for 18 h at 18° C. Cell pellets were collected by centrifugation at 10,000 g and 4° C. for 20 min, resuspended into 10 ml 20 mM phosphate buffer, pH 7.4, containing 0.5M NaCl and 10 mM imidazole and were lysed by sonication. Cell debris was removed by centrifugation at 10,000 g and 4° C. for 30 min and the cleared supernatant immediately applied to 2 ml of nickel-nitrilotriacetic acid (Ni-NTA) resin (QIAgen Valencia, Calif.), pre-equilibrated with the lysis buffer. Protein was allowed to bind for 30 min at 4° C. with gentle agitation, and the resin washed 4 times with 50 ml each lysis buffer. Finally, the enzyme was eluted by incubation of the resin with 2 ml lysis buffer containing 100 mM imidazole for 10 min at 4° C. with gentle agitation. The purified enzyme was applied to a PD-10 desalting column (Amersham Biosciences AB) equilibrated with 50 mM Tris-HCl (pH 8.0) and eluted as described by the manufacturer. Protein aliquots were immediately flash frozen in liquid nitrogen and stored at −80° C. Protein purity was verified by SDS-PAGE. Protein quantification was carried out using the Bradford Protein Assay Kit from Bio-Rad.

### Example 2

General Materials and Methods.

Unless otherwise stated, all chemicals and reagents were purchased from Sigma-Aldrich (St. Louis, Mo., USA) or New England Biolabs (Ipswich, Mass., USA). Compounds 63 and 67 were obtained from Indofine Chemicals (Hillsborough, N.J., USA). Compounds 66, 71, 72, 74 and 83 were obtained from EMD Chemicals (Darmstadt Germany). 75 and 103 were obtained from Fisher Scientific (Pittsburgh, Pa., USA). Compound 76 was obtained from MP Biochemicals (Solon, Ohio, USA). Compounds 73, 79, 90, 96, 99 and 111 were obtained from LC Laboratories (Woburn, Mass., USA). Compound 80 was obtained from Selleck Chemicals (Houston, Tex., USA). Compound 87 was obtained from Toronto Research Chemicals (Toronto, ON, Canada). Compound 91 was previously synthesized. Compound 104 was isolated from fermentation.

General Methods.

High resolution mass spectra were determined on a Bruker MaX is ultra-high resolution quadrupole time of flight mass spectrometer by negative ionization electrospray with a source potential of 2800 V, drying gas at 200° C. flowing at 4 L/min and a nebulizing gas pressure of 0.4 bar. Samples were infused at 3 μL/min and spectra collected for 2 min. Routine TLC analyses were performed on aluminum TLC plates coated with 0.2 mm silica gel (from Sigma-Aldrich, St. Louis, Mo., USA) and monitored at 254 nm.

Flash column chromatography was achieved on 40-63 μm, 60 Å silica gel (from Silicycle, Quebec, Canada). Unless otherwise noted, analytical reverse-phase HPLC was conducted with a Gemini-NX C-18 (5 μm, 250×4.6 mm) column (from Phenomenex, Torrance, Calif., USA) with a gradient of 10% B to 75% B over 20 min, 75% B to 95% B over 1 min, 95% B for 5 min, 95% B to 10% B over 3 min, 10% B for 6 min (A=dH2O with 0.1% TFA; B=acetonitrile; flow rate=1 mL min-1) and detection monitored at 254 nm. Regardless of method, HPLC peak areas were integrated with Star Chromatography Workstation Software (from Varian, Palo Alto, Calif., USA) and the percent conversion calculated as a percent of the total peak area.

NMR spectra were obtained using a UNITYINOVA 400 MHz instrument (from Varian, Palo Alto, Calif., USA) in conjunction with a QN Switchable BB probe (from Varian) or UNITYINOVA 500 MHz instrument in conjunction with a qn6121 probe (from Nalorac, Martinez, Calif., USA). 1H and 13C chemical shifts were referenced to internal solvent resonances. 31P chemical shifts were not referenced. Multiplicities are indicated by s (singlet), d (doublet), t (triplet), q (quartet), qn (quintet), m (multiplet) and br (broad). Italicized elements or groups are those that are responsible for the shifts. Chemical shifts are reported in parts per million (ppm) and coupling constants (J) are given in Hz. NMR assignments were performed with the aid of gCOSY and gHSQC experiments.

Protein Production and Purification.

A single protocol based upon previously published methods for OleD and GtfE was utilized for all purifications. Specifically, single isolates of E. coli BL21(DE3)pLysS (Stratagene, La Jolla, Calif., USA) transformed, with pET28a/oleD, pET28a/oleD[P67T/S132F/A242V] (produces OleD variant ASP), pET28a/oleD[P67T/S132F/A242L] (produces OleD variant 3-1H12), pET28a/oleD[P67T/S132F/A242L/Q268V] (produces OleD variant TDP-16), or pET22b/gtfE vector were utilized for protein production and purification. Briefly, single colonies were used to inoculate 5 mL starter cultures with 50 μg mL-1 kanamycin (for pET28a) or 100 μg mL-1 ampicillin (for pET22b) and incubated overnight at 37° C. and 250 rpm. 4 mL of saturated starter culture was transferred to 1 L cultures of Luria-Bertani medium supplemented with 50 μmL-1 kanamycin (for pET28a) or 100 μg mL-1 ampicillin (for pET22b) and grown at 37° C. until the OD600 reached ^{˜}0.7. Isopropyl β-D-thiogalactoside (0.4 mM final concentration) was added and cultures were incubated at 28° C. for approximately 18 hours at 250 rpm. Cell pellets were collected by centrifugation (6,000 g at 4° C. for 20 min), resuspended in 10 mL of chilled lysis buffer (20 mM phosphate buffer, pH 7.4, 0.5 M NaCl, 10 mM imidazole), and lysed by sonication (5 pulses of 30 seconds each) in an ice bath. Cell debris was removed by centrifugation (10,000 g at 4° C. for 20 min) and the cleared supernatant was incubated with alkaline phosphatase (4 U ml-1; from Roche, Basel, Switzerland) on ice for 2.5 hours with occasional agitation to degrade contaminating nucleotide diphosphates. Following, the supernatant was applied to 2 mL of nickel nitrilotriacetic acid resin (from QIAgen Valencia, Calif., USA) preequilibrated with wash buffer (20 mM phosphate buffer, pH 7.4, 0.3 M NaCl, 10 mM imidazole).

Protein was allowed to bind for 30 min at 4° C. and the resin washed with 4×50 mL wash buffer. Finally, the enzyme was eluted with 2 mL of chilled wash buffer containing an additional 240 mM imidazole for 10 min at 4° C. Purified protein was applied to a PD-10 desalting column (from Amersham Biosciences, Piscataway, N.J., USA), equilibrated with 50 mM Tris-HCl (pH 8.0), and eluted as described by the manufacturer to typically provide 2 mL of desired protein at typical concentrations ranging from 5-12 mg/mL. Final purified proteins were flash frozen drop-wise in liquid nitrogen and stored at −80° C. Protein purity was confirmed by SDS-PAGE to be >95% and protein concentration was determined using the Bradford Protein Assay Kit (from Bio-Rad, Hercules, Calif., USA). Small aliquots of protein were thawed for experiments as required and did not undergo multiple freeze/thaw cycles.

### Syntheses of β-D-glucosides (1-32)

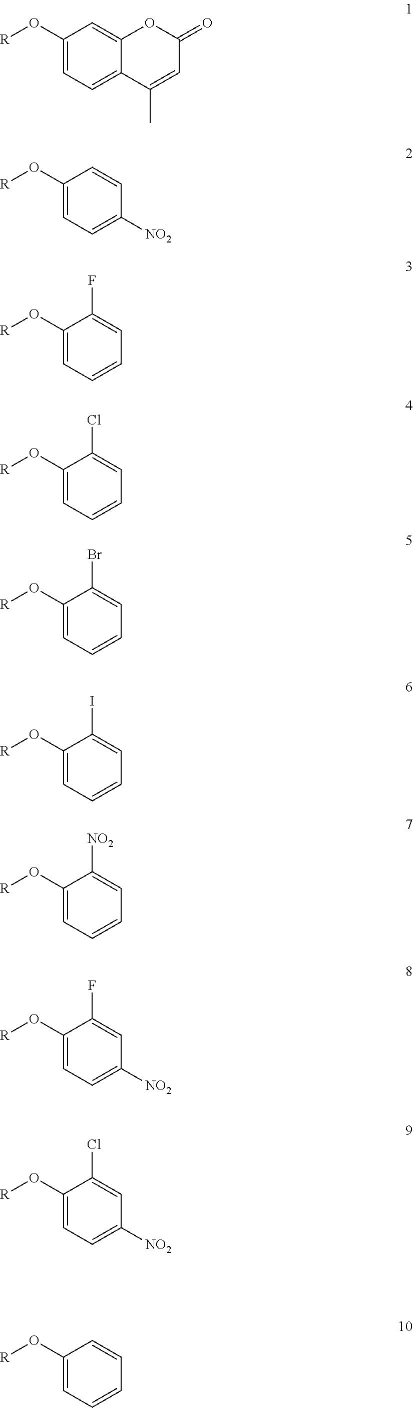

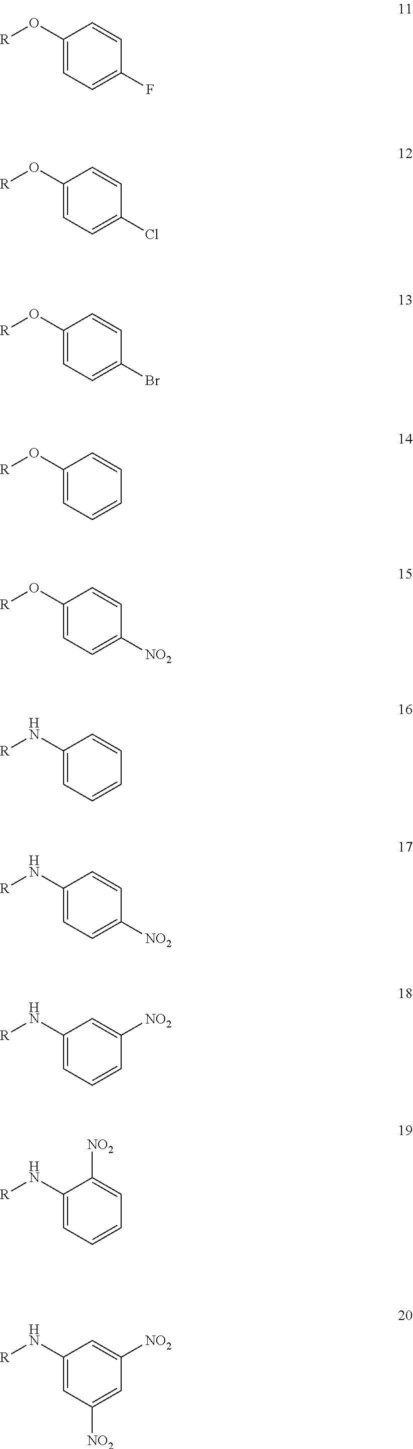

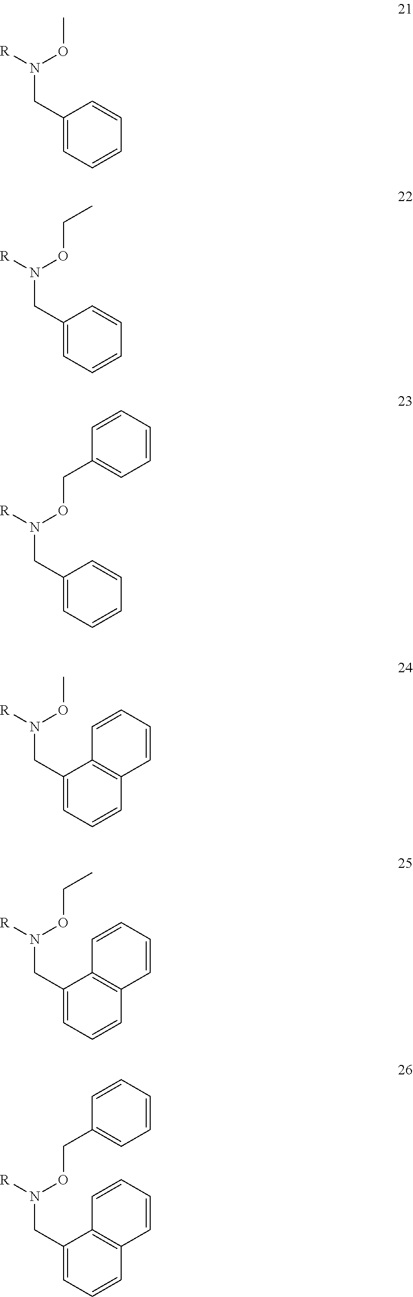

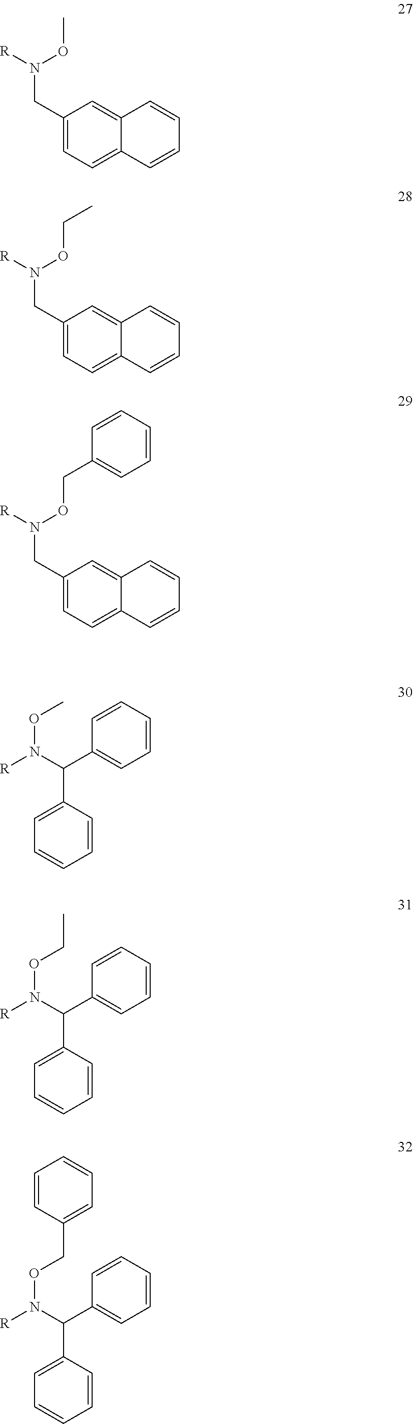

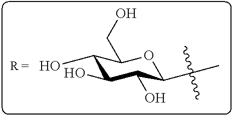

Substituted O-phenyl-β-D-glucosides (3-6, 8, 11-13).

According to a procedure from Lee, et al., penta-O-acetyl-β-Dglucose (1 equiv.) and substituted phenol (2 equiv.) were added to a round bottom flask flushed with argon. Triethylamine (1 equiv.) in 9 mL of anhydrous CH₂Cl₂ was added. Boron trifluoride diethyl etherate (5 equiv.) in 1 mL of anhydrous CH₂Cl₂ was added dropwise to the reaction over 30 minutes. The mixture was kept under argon and allowed to proceed at room temperature. After the reaction was determined to be complete by TLC, an equal volume of saturated aqueous NaHCO3 was added and the reaction was stirred until the evolution of gas halted. The organic layer was recovered and the aqueous layer was extracted 2× with an equal volume of CH₂Cl₂. The combined organic layers were dried over sodium sulfate and concentrated with reduced pressure. The peracetylated intermediate was purified by flash chromatography with EtOAc/Hexanes (1:2).

General Deprotection Procedure.

The purified peracetylated glucoside intermediate was dissolved in MeOH (20 mL mmol-1), a catalytic amount of sodium methoxide powder was added, and the reaction was allowed to proceed overnight with stirring at room temperature. Neutralization was then performed by adding Amberlite IR-120 (H+ form) ion-exchange resin (from Sigma Aldrich, St. Louis, Mo., USA). The resin was filtered off using a small column of Celite 545 (from Fisher Scientific, Pittsburgh, Pa., USA), and then concentrated with reduced pressure to yield the final product without further purification.

### Synthesis of 2-fluorophenyl-β-D-glucopyranoside (3)

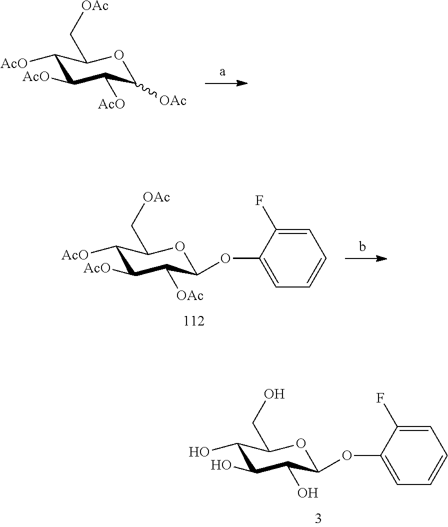

a) 2-fluorophenol, BF3.OEt2, Et3N in CH2Cl2, rt, 36 h; b) NaOMe 0.1M in MeOH, rt, 18 h.

(2-fluorophenyl)-2,3,4,6-tetra-O-acetyl-β-D-glucopyranoside (112).

General procedure above with 2-fluorophenol (0.22 g, 2.0 mmol) yielded 112 (0.37 g, 83% yield) as a white solid in a 36 hour reaction. TLC Rf=0.15 (EtOAc/Hexanes, 1:3); MS-ESI (m/z): [M−H]− calcd for C₂₀H₂₂FO₁₀, 441.1; found 441.1.

2-fluorophenyl-β-D-glucopyranoside (3). General procedure with 112 (0.37 g, 0.83 mmol) yielded 3 (0.21 g, 93% yield) as white crystals. TLC Rf=0.22 (CH2Cl2/MeOH, 9:1); 1H NMR (400 MHz, CD₃OD) δ 7.27 (dt, J_{Ph, Ph}=1.6 Hz, J_{Ph, Ph}=8.3 Hz, 1H, Ph), 7.14-7.05 (m, 2H, Ph), 7.02-6.95 (m, 1H, Ph), 4.96 (d, J_{H1, H2}=7.6 Hz, 1H, H-1), 3.87 (dd, JH5, H6a=1.8 Hz, J_{H6a, H6b}=12.1 Hz, 1H, H-6a), 3.70 (dd, J_{H5, H6b}=5.2 Hz, 1H, H-6b), 3.53-3.37 (m, 4H, H-2, H-3, H-4, H-5); 13C NMR (100 MHz, CD₃OD) δ 155.0 (d, 1JC, F=244.0 Hz), 146.6 (d, 2JC, F=10.3 Hz), 125.6 (d, 3JC, F=4.0 Hz), 123.9 (d, 3JC, F=7.4 Hz), 119.3, 117.2 (d, 2JC, F=19.2 Hz), 102.7, 78.3, 78.0, 74.9, 71.3, 62.5; HRMS (m/z): [M+Na]+ calcd for C₁₂H₁₅FNaO₆ 297.0745; found 297.0751.

### Synthesis of 2-chlorophenyl-β-D-glucopyranoside (4)

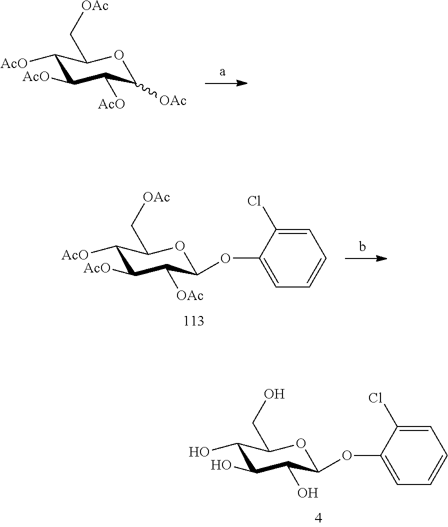

a) 2-chlorophenol, BF3.OEt2, Et3N in CH2Cl2, rt, 36 h; b) NaOMe 0.1M in MeOH, rt, 18 h.

(2-chlorophenyl)-2,3,4,6-tetra-O-acetyl-β-D-glucopyranoside (113). General procedure with 2-chlorophenol (0.26 g, 2.0 mmol) yielded 113 (0.29 g, 62% yield) as a white powder in a 36 hour reaction. TLC Rf=0.13 (EtOAc/Hexanes, 1:3); MS-ESI (m/z): [M−H]−calcd for C20H22ClO10, 457.1; found 457.2.

2-chlorophenyl-β-D-glucopyranoside (4).

General procedure with 113 (0.29 g, 0.62 mmol) yielded 4 (0.18 g, 99% yield) as white crystals. TLC Rf=0.20 (CH₂Cl₂/MeOH, 9:1); 1H NMR (400 MHz, CD₃OD) δ 7.35 (dd, J_{Ph, Ph}=1.5 Hz, J_{Ph, Ph}=8.0 Hz, 1H, Ph), 7.29-7.21 (m, 2H, Ph), 7.01-6.95 (m, 1H, Ph), 4.99 (d, J_{H1, H2}=7.5 Hz, 1H, H-1), 3.88 (dd, JH5, H6a=2.0 Hz, J_{H6a, H6b}=12.1 Hz, 1H, H-6a), 3.70 (dd, J_{H5, H6b}=5.2 Hz, 1H, H-6b), 3.56-3.50 (m, 1H, H-2), 3.50-3.40 (m, 3H, H-3, H-4, H-5); 13C NMR (100 MHz, CD₃OD) δ 153.2, 130.0, 127.8, 123.2, 122.8, 116.7, 101.0, 77.1, 76.9, 73.6, 70.0, 61.2; HRMS (m/z): [M+Na]+ calcd for C12H15CINaO6; 313.0450; found 313.0456.

### Synthesis of 2-bromophenyl-β-D-glucopyranoside (5)

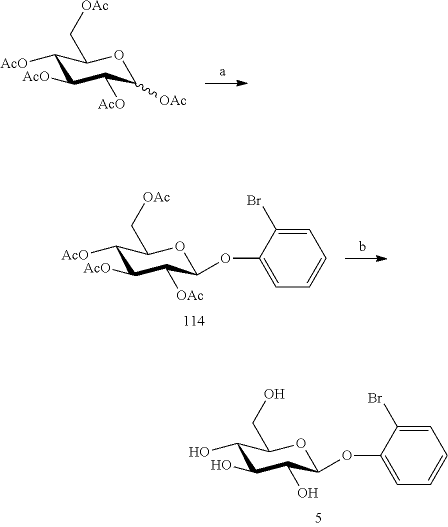

a) 2-bromophenol, BF3.OEt2, Et3N in CH2Cl2, rt, 48 h; b) NaOMe 0.1M in MeOH, rt, 18 h.

(2-bromophenyl)-2,3,4,6-tetra-O-acetyl-β-D-glucopyranoside (114).

General procedure with 2-bromophenol (0.35 g, 2.0 mmol) yielded 114 (0.24 g, 47% yield) as a white powder in a 48 hour reaction. TLC Rf=0.10 (EtOAc:Hexanes, 1:3); MS-ESI (m/z): [M−H]− calcd for C20H22BrO10, 501.0; found 501.1.

2-bromophenyl-β-D-glucopyranoside (5).

General procedure with 114 (0.24 g, 0.47 mmol) yielded 5 (0.16 g, 99% yield) as white crystals. TLC Rf=0.25 (CH₂Cl₂/MeOH, 9:1); 1H NMR (400 MHz, CD₃OD) δ 7.52 (dd, J_{Ph, Ph}=1.5 Hz, J_{Ph, Ph}=8.0 Hz, 1H, Ph), 7.31-7.20 (m, 2H, Ph), 6.94-6.87 (m, 1 H, Ph), 4.99 (d, J_{H1, H2}=7.6 Hz, 0.93H, H-1), 3.87 (dd, JH5, H6a=2.0 Hz, J_{H6a, H6b}=12.1 Hz, 1H, H-6a), 3.69 (dd, J_{H5, H6b}=5.1 Hz, 1H, H-6b), 3.53 (dd, JH2, H3=8.8 Hz, 1H, H-2), 3.49-3.35 (m, 3H, H-3, H-4, H-5); 13C NMR (100 MHz, CD₃OD) δ 154.2, 133.1, 128.5, 123.2, 116.5, 112.2, 101.0, 77.1, 76.9, 73.6, 70.0, 61.2; HRMS (m/z): [M+NH4]+ calcd for C₁₂H₁₉BrNO₆; 352.0391; found 352.0386.

### Synthesis of 2-iodophenyl-β-D-glucopyranoside (6)

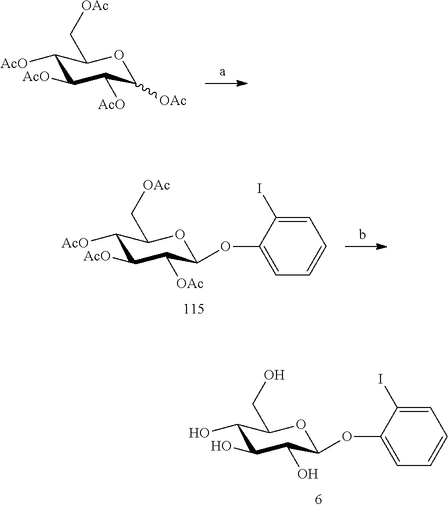

a) 2-iodophenol, BF3.OEt2, Et3N in CH2Cl2, rt, 36 h; b) NaOMe 0.1M in MeOH, rt, 18 h.

(2-iodophenyl)-2,3,4,6-tetra-O-acetyl-β-D-glucopyranoside (115).

General procedure with 2-iodophenol (0.30 g, 2.0 mmol) yielded 115 (0.27 g, 53% yield) as a white powder in a 36 hour reaction. TLC Rf=0.15 (EtOAc/Hexanes, 1:3); MS-ESI (m/z): [M−H]−calcd for C₂₀H₂₂IO₁₀, 549.0; found 549.0.

2-iodophenyl-β-D-glucopyranoside (6).

General procedure with 115 (0.27 g, 0.49 mmol) yielded 6 (0.15 g, 84% yield) as white crystals. TLC Rf=0.27 (10% MeOH in CH₂Cl₂); 1H NMR (400 MHz, CD₃OD) δ 7.76 (dd, J_{Ph, Ph}=1.6 Hz, J_{Ph, Ph}=8.0 Hz, 1H, Ph); 7.33-7.28 (m, 1H, Ph), 7.16 (dd, J_{Ph, Ph}=1.6 Hz, J_{Ph, Ph}=8.0 Hz, 1H, Ph), 6.78-6.74 (m, 1H, Ph), 5.00 (d, JH-1b, H-2=7.6 Hz, 0.9H, H-1), 3.88 (dd, JH-6a, H-5=2.0 Hz, JH-6a, H-6b=12.1 Hz, 1H, H-6a), 3.69 (dd, JH-6b, H-5=5.4 Hz, JH-6b, H-6a=12.1 Hz, 1H, H-6b), 3.55 (m, 1H, H-2), 3.49-3.37 (m, 3H, H-3, H-4, H-5); 13C NMR (100 MHz, CD₃O) δ 156.6, 139.4, 129.4, 123.7, 115.3, 101.1, 86.0, 77.1, 77.0, 73.7, 70.0, 61.3; HRMS (m/z): [M+NH₄]+ calcd for C₁₂H₁₉INO₆, 400.0252; found 400.0246. 1 Less than 10% of α-anomer was observed.

### Synthesis of 2-fluoro-4-nitrophenyl-β-D-glucopyranoside (8)

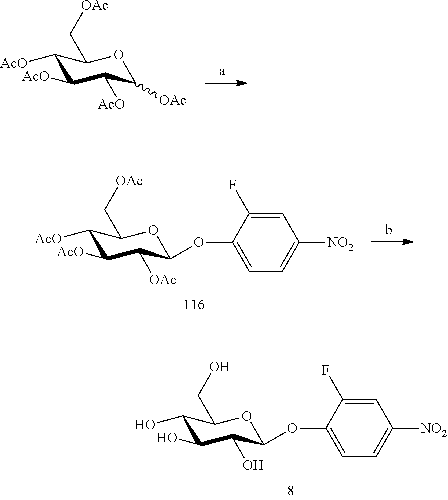

a) 2-fluoro-4-nitrophenol, BF3.OEt2, Et3N in CH2Cl2, rt, 36 h; b) NaOMe 0.1M in MeOH, rt, 18 h.

(2-fluoro-4-nitrophenyl)-2,3,4,6-tetra-O-acetyl-β-D-glucopyranoside (116).

General procedure with 2-fluoro-4-nitrophenol (0.31 g, 2.0 mmol) yielded 116 (0.35 g, 72% yield) as a white solid in a 36 hour reaction. TLC Rf=0.19 (EtOAc/Hexanes, 1:3); MS-ESI (m/z): [M]⁻ calcd for C₂₀H₂₂FNO₁₂, 487.1; found 487.1.

2-fluoro-4-nitrophenyl-β-D-glucopyranoside (8).

General procedure with 116 (0.35 g, 0.73 mmol) yielded 8 (0.13 g, 40% yield) as yellow crystals. TLC Rf=0.16 (CH₂Cl₂/MeOH, 9:1); 1H NMR (400 MHz, CD₃OD) δ 8.10-8.03 (m, 2H, Ph), 7.49-7.43 (m, 1H, Ph), 5.15 (d, J_{H1, H2}=7.4 Hz, 1H, H-1), 3.90 (dd, JH5, H6a=3.9 Hz, J_{H6a, H6b}=12.1 Hz, 1H, H-6a), 3.70 (dd, J_{H5, H6b}=5.7 Hz, 1H, H-6b), 3.57-3.37 (m, 4H, H-2, H-3, H-4, H-5); 13C NMR (100 MHz, CDCl₃) δ 153.0 (d, 1JC, F=250.8 Hz), 152.2 (d, 2JC, F=10.5 Hz), 143.4 (d, 3JC, F=7.5 Hz), 121.8 (d, 3JC, F=3.5 Hz), 117.9, 113.2 (d, 2JC, F=23.4 Hz), 102.0, 78.6, 80.0, 74.6, 71.1, 62.4; HRMS (m/z): [M+Na]+ calcd for C₁₂H₁₄FNNaO₈, 342.0596; found 342.0606.

### Synthesis of 4-fluorophenyl-β-D-glucopyranoside (11)

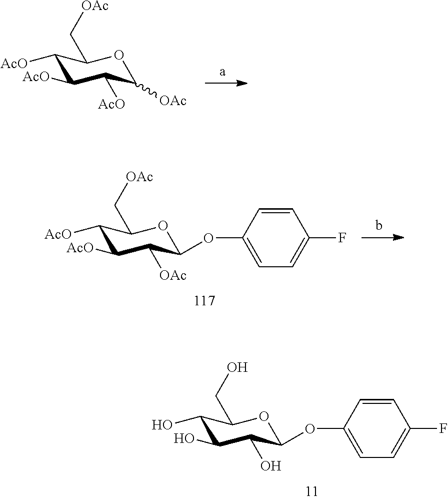

a) 4-fluorophenol, BF3.OEt2, Et3N in CH2Cl2, rt, 30 h; b) NaOMe 0.1M in MeOH, rt, 18 h.

(4-fluorophenyl)-2,3,4,6-tetra-O-acetyl-β-D-glucopyranoside (117).

General procedure with 4-fluorophenol (0.22 g, 2.0 mmol) yielded 117 (0.38 g, 87% yield) as a white powder in a 30 hour reaction. TLC Rf=0.17 (EtOAc:Hexanes, 1:3); MS-ESI (m/z): calcd for C20H22FO10, 441.1; found 441.1.

4-fluorophenyl-β-D-glucopyranoside (11).

General procedure with 117 (0.38 g, 0.87 mmol) yielded 11 (0.23 g, 96% yield) as pale yellow crystals. TLC Rf=0.23 (MeOH/CH₂Cl₂, 1:9); 1H NMR (400 MHz, CD₃OD) δ 7.15-7.07 (m, 2H, Ph), 7.05-6.97 (m, 2H, Ph), 4.83 (d, J_{H1, H2}=7.6 Hz, 1H, H-1), 3.90 (dd, J_{H5, H6a}=2.0 Hz, J_{H6a, H6b}=12.1 Hz, 1H, H-6a), 3.71 (dd, J_{H5, H6b}=5.3 Hz, 1H, H-6b), 3.51-3.36 (m, 4 H, H-2, H-3, H-4, H-5); 13C NMR (100 MHz, CD₃OD) δ 159.5 (d, 1JC, F=237.2 Hz), 155.4 (d, 4JC, F=2.3 Hz), 119.4 (d, 3JC, F=8.1 Hz), 116.7 (d, 2JC, F=23.4 Hz), 103.1, 78.2, 78.0, 74.9, 71.4, 62.6; HRMS-ESI (m/z): [M+NH₄]+ calcd for C₁₂H₁₉FNO₆, 292.1191; found 292.1193.

### Synthesis of 4-chlorophenyl-β-D-glucopyranoside (12)

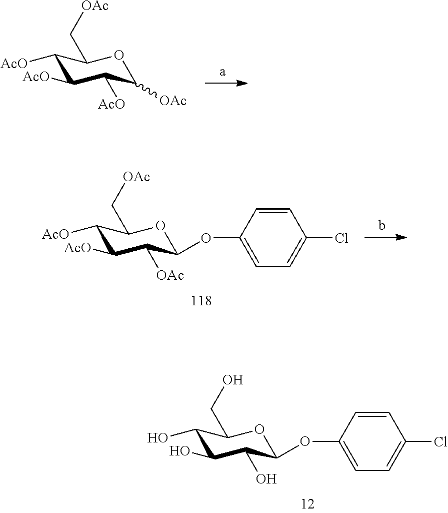

a) 4-chlorophenol, BF3.OEt2, Et3N in CH2Cl2, rt, 30 h; b) NaOMe 0.1M in MeOH, rt, 18 h.

(4-chlorophenyl)-2,3,4,6-tetra-O-acetyl-β-D-glucopyranoside (118).

General procedure with 4-chlorophenol (0.26 g, 2.0 mmol) yielded 118 (0.37 g, 82% yield) as white crystals in a 30 hour reaction. TLCRf=0.13 (EtOAc/Hexanes, 1:3); MS-ESI (m/z): [M−H]−calcd for C₂₀H₂₂ClO₁₀, 457.0; found 457.0.

4-chlorophenyl-β-D-glucopyranoside (12).

General procedure with 118 (0.37 g, 0.82 mmol) yielded 12 (0.23 g, 99% yield) as white crystals. TLC Rf=0.24 (CH₂Cl₂/MeOH, 9:1); 1H NMR (400 MHz, CD₃OD) δ 7.30-7.24 (m, 2H, Ph), 7.11-7.05 (m, 2H, Ph), 4.88 (d, J_{H1, H2}=7.2 Hz, 1H, H-1), 3.90 (dd, J_{H5, H6a}=2.0 Hz, J_{H6a, H6b}=12.1 Hz, 1H, H-6a), 3.70 (dd, J_{H5, H6b}=5.4 Hz, 1H, H-6b), 3.52-3.36 (m, 4H, H-2, H-3, H-4, H-5); 13C NMR (100 MHz, CD₃OD) δ 157.9, 130.3, 128.3, 119.4, 102.5, 78.2, 78.0, 74.9, 71.4, 62.5; HRMS (m/z):[M+NH₄]+ calcd for C₁₂H₂₉ClNO₆, 308.0896; found 308.0902.

### Synthesis of 4-bromophenyl-β-D-glucopyranoside (13)

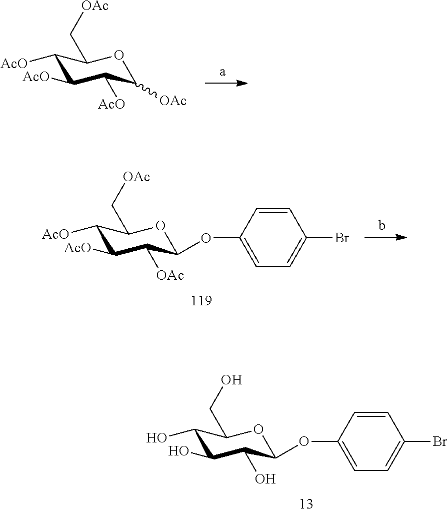

a) 4-bromophenol, BF₃.OEt₂, Et₃N in CH₂Cl₂, rt, 17 h; b) NaOMe 0.1M in MeOH, rt, 18 h.

(4-bromophenyl)-2,3,4,6-tetra-O-acetyl-β-D-glucopyranoside (119).

General procedure with 4-bromophenol (0.35 g, 2.0 mmol) yielded 119 (0.09 g, 20.3% yield) as a white powder in a 17 hour reaction. TLC Rf=0.13 (EtOAc:Hexanes, 1:3); MS-ESI (m/z): [M−H]− calcd for C₂₀H₂₂BrO₁₀, 501.0; found 501.1.

4-bromophenyl-β-D-glucopyranoside (13).

General procedure with 119 (0.09 g, 0.2 mmol) yielded 13 (0.07 g, 99% yield) as pale yellow crystals. TLC Rf=0.24 (CH₂Cl₂/MeOH, 9:1); 1H NMR (400 MHz, CD₃OD) δ 7.44-7.38 (m, 2H, Ph), 7.06-7.00 (m, 2H, Ph), 4.88 (d, J_{H1, H2}=7.6 Hz, 1H, H-1), 3.90 (dd, J_{H5, H6a}=2.1 Hz, J_{H6a, H6b}=12.1 Hz, 1H, H-6a), 3.70 (dd, J_{H5, H6b}=5.4 Hz, 1H, H-6b), 3.53-3.36 (m, 4H, H-2, H-3, H-4, H-5); 13C NMR (100 MHz, CD₃OD) δ 158.37, 133.3, 119.8, 115.6, 102.4, 78.3, 78.0, 74.9, 71.4, 62.5; [M+NH₄]+ calcd for C₁₂H₂₉BrNO₆, 352.0391; found 352.0393.

### Synthesis of S-phenyl-1-thio-β-D-glucopyranoside (14)

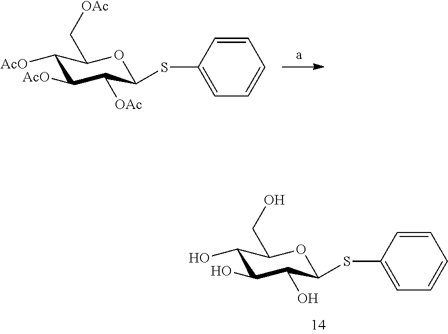

### a) NaOMe 0.1M in MeOH, rt, 90 min.

S-phenyl-1-thio-β-D-glucopyranoside tetracetate (1.02 g, 2.31 mmol) was dissolved in MeOH (7.57 mL) to a final concentration of 300 mM. The solution was stirred on an ice bath and then sodium methoxide powder (1.25 g, 23.1 mmol) dissolved in 7.57 mL of MeOH was added to the reaction and allowed to proceed for 1.5 hours. Compound 14 (0.51 g, 81% yield) was purified by flash chromatography with a 1% to 20% gradient of MeOH in CH₂Cl₂. TLC Rf=0.12 (CH₂Cl₂/MeOH, 9:1); 1H NMR (500 MHz, CD₃OD) δ 7.60-7.57 (m, 2H, Ph), 7.35-7.30 (m, 2H, Ph), 7.29-7.26 (m, 1H, Ph), 4.63 (d, J_{H1, H2}=9.8 Hz, 1H, H-1), 3.89 (dd, J_{H6a, H6b}=12.1 Hz, JH5, H6a=1.8 Hz, 1H, H-6a), 3.70 (dd, J_{H5, H6b}=5.3 Hz, 1H, H-6b), 3.42 (dd, J_{H2, H3}=8.6 Hz, J_{H3, H4}=8.6 Hz, 1H, H-3), 3.38-3.30 (m, 2H, H-4, H-5), 3.25 (dd, 1H, H-2); 13C NMR (125 MHz, CD₃OD) δ 135.21, 132.6, 129.8, 128.3, 89.3, 82.0, 79.6, 73.7, 71.3, 62.8; HRMS-ESI (m/z): [M+Na]+ calcd for C₁₂H₁₆NaO₅S, 295.06107; found 295.06113.

### Synthesis of S-(4-nitrophenyl)-1-thio-β-D-glucopyranoside (15)

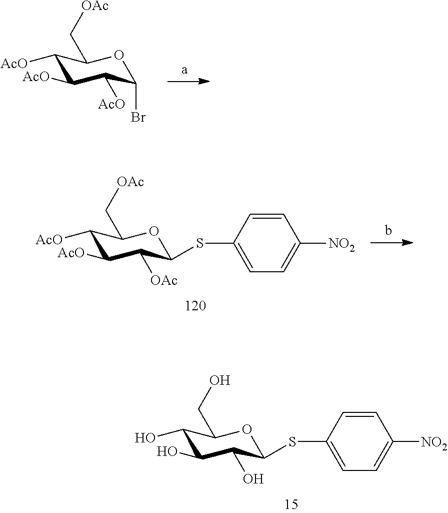

a) 4-nitrothiophenol, tetrabutylammonium bisulfate, Na₂CO₃, EtOAc, rt, 18 h; b) NaOMe 0.1M in MeOH, rt, 8 h.

S-(4-nitrophenyl)-1-thio-2,3,4,6-tetra-O-acetyl-β-D-glucopyranoside (120).

Using a modified protocol described by D. Carrière et al., 2,3,4,6-tetra-O-acetyl-α-D-bromoglucose (0.1 g, 0.24 mmol) was dissolved in 3 mL of EtOAc. Tetrabutylammonium bisulfate (83 mg, 0.24 mmol), 4-nitrothiophenol (0.19 g, 1.22 mmol) and 3 mL of a 1M solution of Na₂CO₃ were successively added, yielding 120 (0.03 g, 25% yield) as white crystals. TLC Rf=0.11 (EtOAc/Hexanes, 1:3); 1H NMR (400 MHz, CDCl₃) □ 8.16 (d, J_{Ph, Ph}=9.0 Hz, 2H, Ph), 7.60 (d, J_{Ph, Ph}=9.0 Hz, 2H, Ph), 5.28 (t, J=9.4 Hz, 1H, H-3), 5.12-5.02 (m, 2H, H-2, H-4), 4.88 (d, J_{H1, H2}=10.0 Hz, 1H, H-1), 4.26 (dd, J_{H5, H6a}=5.6 Hz, J_{H6a, H6b}=12.3 Hz, 1H, H-6a), 4.20 (dd, J_{H5, H6b}=2.4 Hz, 1H, H-6b), 3.87-3.80 (m, 1H, H-5), 2.11 (s, 3H, OCH₃), 2.09 (s, 3 H, OCH₃), 2.05 (s, 3H, OCH₃), 2.01 (s, 3H, OCH₃); 13C NMR (100 MHz, CDCl₃) □ 170.5, 170.2, 169.5, 169.3, 147.2, 141.8, 131.2, 124.0, 84.5, 76.2, 73.3, 69.7, 68.1, 62.2, 20.9, 20.8, 20.7; HRMS-ESI (m/z): [M+Na]+ calcd for C₂₀H₂₃NNaO₁₁S, 508.0885; found 508.0880.

S-(4-nitrophenyl)-1-thio-β-D-glucopyranoside (15).

Compound 120 (0.03 g, 0.06 mmol) was dissolved in MeOH (20 mL mmol-1), sodium methoxide powder (4.9 mg, 0.09 mmol) was added, and the reaction allowed to proceed for 8 hours. The reaction was filtered over a small column of Celite 545 (Fisher Scientific, Pittsburgh, Pa., USA) to yield 15 (0.017 g, 90% yield) as a yellow solid. TLC Rf=0.44 (15% MeOH in CH₂Cl₂); 1H NMR (400 MHz, DMSO-d6) δ 8.12 (d, J_{Ph, Ph}=9.0 Hz, 2H, Ph), 7.62 (d, J_{Ph, Ph}=9.0 Hz, 2H, Ph), 5.68 (br s, 1H, OH), 5.50 (br s, 1H, OH), 5.28 (br s, 1H, OH), 4.92 (d, J_{H1, H2}=9.6 Hz, 1H, H-1), 4.66 (br s, 1H, OH), 3.71 (d, J_{H6a, H6b}=11.6 Hz, 1H, H-6a), 3.47 (dd, J_{H5, H6b}=5.6 Hz, 1H, H-6b), 3.42-3.24, (m, 2H, H-3, H-5), 3.22-3.12 (m, 2H, H-2, H-4); 13C NMR (100 MHz, DMSO-d6) δ 146.2, 144.9, 127.7, 123.8, 85.1, 81.1, 78.1, 72.5, 69.6, 60.8; HRMS-ESI (m/z): [M+Na]+ calcd for C12H15NNaO7S, 340.0462; found 340.0467.

### Substituted N-phenyl-β-D-glucosylamines (16-20)

### Synthesis of N-phenyl-D-glucopyranosylamine (16)

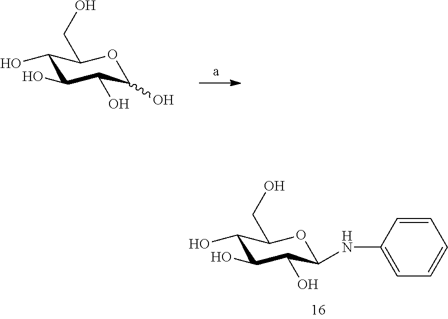

a) aniline, 100 mM sodium phosphate buffer (pH 6.5), 40° C., 5 h.

This compound was synthesized as previously described, and yielded 16 (0.43 g, 58% yield) as a white solid. TLC Rf=0.28 (CH₂Cl₂/MeOH, 8:2); 1H NMR (500 MHz, CD₃OD) δ 7.16 (dd, J_{Ph, Ph}=7.4 Hz, J_{Ph, Ph}=8.4 Hz, 2H, Ph), 6.82 (d, J_{Ph, Ph}=7.4 Hz, 2H, Ph), 6.74 (t, J_{Ph, Ph}=7.4 Hz, 1H, Ph), 4.58 (d, J_{H1, H2}=8.7 Hz, 1H, H-1), 3.88 (dd, J_{H6a, H6b}=11.7, J_{H5, H6a}=1.1 Hz, 1H, H-6a), 3.73-3.68 (m, 1H, H-6b), 3.54-3.49 (m, 1H, H-5), 3.42-3.32 (m, 3H, H-2, H-3, H-4); 13C NMR (125 MHz, CD₃OD) δ 148.0, 130.0, 119.5, 115.1, 86.8 (C-1), 79.0 (C-5), 78.3 (C-3), 74.6 (C-2), 71.7 (C-4), 62.6 (C-6). Spectral data were consistent with those previously reported (59). HRMS-ESI (m/z): [M+Na]+ calcd for C₁₂H₁₇NNaO₅, 278.09989; found 278.1001.

### Synthesis of N-(4-nitrophenyl)-β-D-glucopyranosylamine (17)

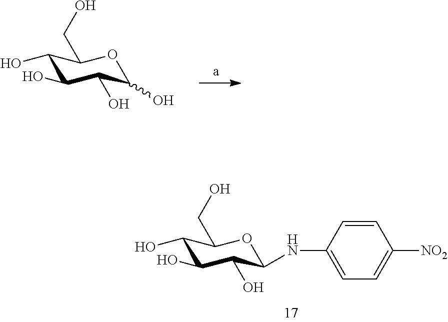

a) 4-nitroaniline, H₂SO₄ (1M) in MeOH, 50° C., 2 h.

A quantity of 4-nitroaniline (0.15 g, 1.1 mmol) was dissolved to a final concentration of 250 mM in MeOH and heated to 50° C. D-glucose (0.13 g, 0.7 mmol) and then 3 μL of concentrated sulfuric acid were added and the reaction was allowed to proceed for 2 hours. A small scoop of sodium bicarbonate was added and the reaction was filtered. Following filtration, the desired product was recrystallized from the recovered filtrate. The resulting crystals were stored at −80° C. for 4 hours, washed with diethyl ether, dissolved with MeOH, and concentrated via reduced pressure to yield 17 (0.05 g, 21% yield) as yellow crystals. TLC Rf=0.30 (15% MeOH in CH₂Cl₂); 1H NMR (400 MHz, D₂O) δ 8.13 (d, J_{Ph, Ph}=9.2 Hz, 2H, Ph), 6.89 (d, J_{Ph, Ph}=9.2 Hz, 2H, Ph), 4.86 (d, J_{H1, H2}=8.7 Hz, 1H, H-1), 3.91 (dd, J_{H5, H6a}=2.1 Hz, J_{H6a, H6b}=12.3 Hz, 1H, H-6a), 3.74 (dd, J_{H5, H6b}=5.5 Hz, 1H, H-6b), 3.66-3.58 (m, 2H, H-3, H-5), 3.54-3.44 (m, 2H, H-2, H-4); 13C NMR (100 MHz, D₂O) δ 153.2, 139.4, 127.1, 113.6, 84.0, 77.3, 73.0, 70.1, 61.2; HRMS-ESI (m/z): [M+Na]+ calcd for C12H16N2NaO7, 323.0850; found 323.0844.

### Synthesis of N-(3-nitrophenyl)-β-D-glucopyranosylamine (18)

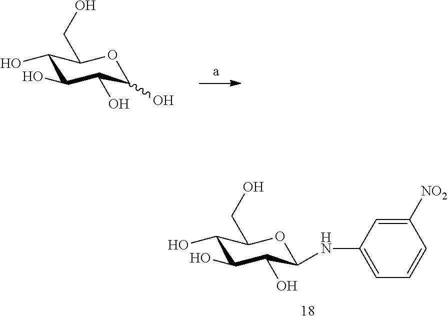

a) 3-nitroaniline, H₂SO₄ (1M) in MeOH, 70° C., 3 h.

Utilizing sulfuric acid instead of glacial acetic acid as previously described, yielded 18 (0.09 g, 17% yield) as bright yellow crystals. TLC Rf=0.32 (15% MeOH in CH₂Cl₂); 1H NMR (500 MHz, CD₃OD) δ 7.60 (t, J_{Ph, Ph}=2.2 Hz, 1H, Ph), 7.53 (ddd, J_{Ph, Ph}=0.8 Hz, J_{Ph, Ph}=2.2 Hz, J_{Ph, Ph}=8.1 Hz, 1H, Ph), 7.33 (t, J_{Ph, Ph}=8.1 Hz, 1H, Ph), 7.13 (ddd, J_{Ph, Ph}=0.8 Hz, J_{Ph, Ph}=2.2 Hz, J_{Ph, Ph}=8.1 Hz, 1H, Ph), 4.61 (d, J_{H1, H2}=8.7 Hz, 1H, H-1), 3.86 (dd, J_{H5, H6a}=2.3 Hz, J_{H6a, H6b}=12.0 Hz, 1H, H-6a), 3.69 (dd, J_{H5, H6b}=5.3 Hz, 1H, H-6b), 3.51-3.47 (m, 1H, H-3), 3.46-3.40 (m, 1H, H-5), 3.39 (m, 2H, H-4, H-2); 13C NMR (125 MHz, CD₃OD) δ 150.5, 149.6, 130.8, 120.8, 113.5, 109.0, 86.2, 79.1, 78.6, 74.5, 71.6, 62.6; HRMS-ESI (m/z): [M+Na]+ calcd for C₁₂H₁₆N₂NaO₇, 323.0850; found 323.0844.

### Synthesis of N-(2-nitrophenyl)-D-glucopyranosylamine (19)

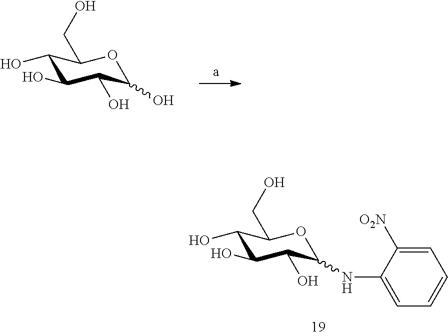

a) 2-nitroaniline, H₂SO₄ (1M) in MeOH, 40° C., 1 h.

A quantity of 2-nitroaniline (0.15 g, 1.1 mmol) was dissolved to a final concentration of 250 mM in MeOH and heated to 40° C. D-glucose (0.130 g, 0.72 mmol) was added and then 36 μL of 1 M H₂SO₄ in MeOH (36 mmol) was added over 1 hour. The reaction was concentrated and then purified by flash chromatography with a 10% to 15% gradient of MeOH in CH₂Cl₂ to yield 19 (0.04 g, 17% yield) as yellow crystals with α- and β-anomers present in a 1:2 ratio. TLC Rf=0.48 (10% MeOH in CH₂Cl₂); 1H NMR (400 MHz, CD₃OD) δ 8.16 (t, J_{Ph, Ph}=8.0 Hz, 1H, Ph), 7.55 (t, J_{Ph, Ph}=8.0 Hz, 1H, Ph), 7.23 (d, J_{Ph, Ph}=8.0 Hz, 1H, Ph), 6.84 (t, J_{Ph, Ph}=8.0 Hz, 1H, Ph), 5.30 (d, J_{H1α, H2}=4.6 Hz, 1H, H1a), 4.72 (d, J_{H1β, H2}=8.4 Hz, 1H, H-1P), 3.89 (dd, J_{H5, H6a}=1.9 Hz, J_{H6a, H6b}=11.9 Hz, 1H, H-6a), 3.82-3.66 (m, 2H, H-6b, H-3), 3.54-3.35 (m, 3H, H-2, H-4, H-5); HRMS-ESI (m/z): [M+Na]+ calcd for C₁₂H₁₆N₂NaO₇, 323.0850; found 323.0858.

### Synthesis of N-(3,5-dinitrophenyl)-β-D-glucopyranosylamine (20)

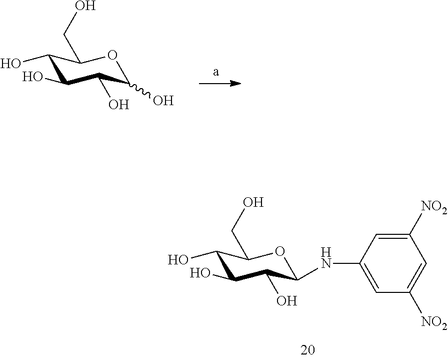

a) 3,5-dinitroaniline, H2SO4 (1M) in MeOH, 50° C., 2 h.

A quantity of 3,5-dinitroanline (0.4 g, 2.2 mmol) was dissolved to a final concentration of 250 mM in MeOH and heated to 50° C. D-glucose (0.39 g, 2.2 mmol) and then 3 μL of concentrated sulfuric acid were added and the reaction was allowed to proceed for 2 hours. The desired product recrystallized from solution as the reaction proceeded. The resulting crystals were stored at −80° C. for 4 hours, washed with diethyl ether, dissolved with MeOH, and concentrated with reduced pressure to yield 20 (0.21 g, 28% yield) as yellow crystals. TLC Rf=0.36 (15% MeOH in CH₂Cl₂); 1H NMR (400 MHz, CD₃OD) δ 8.25 (t, J_{Ph, Ph}=2.0 Hz, 1H, Ph), 7.92 (d, J_{Ph, Ph}=2.0 Hz), 2H, Ph), 4.67 (d, J_{H1, H2}=8.6 Hz, 1H, H-1), 3.88 (dd, J_{H5, H6a}=2.4 Hz, J_{H6a, H6b}=12.0 Hz, 1H, H-6a), 3.68 (dd, J_{H5, H6b}=5.8 Hz, 1H, H-6b), 3.53-3.45 (m, 2 H, H-3, H-5), 3.43-3.34 (m, 2H, H-2, H-4); 13C NMR (100 MHz, CD₃OD) δ 150.74, 150.65, 114.0, 107.6, 85.7, 79.1, 78.9, 74.5, 71.6; HRMS-ESI (m/z): [M+Na]+ calcd for C₁₂H₁₅N₃NaO₉, 368.07005; found 368.06973.

O-substituted oxyamines (122, 124, 126, 128, 130, 132, 134, 136, 138, 140, 142, 144).

General Reductive Amination Procedure.

Aldehyde was dissolved in CH₂Cl₂ to a final concentration of 0.45 M. Unless noted, to this was added 1.5 equivalents of the appropriate O-substituted oxyamine hydrochloride salt and 2.2 equivalents of pyridine. The mixture was stirred for 2 hours at room temperature. TLC analysis revealed the substrate to be completely consumed with two products (presumably E- and Z-oximes) being formed. The reaction mixture was subsequently washed with 5% aqueous HCl (3×50 mL) and saturated NaCl (2×50 mL). The resulting organic layer was dried over Na₂SO₄ and concentrated under reduced pressure to provide the crude oxime which was used in subsequent reactions without further purification.

Crude oxime was dissolved in EtOH to a final concentration of 1.5 M. The reaction mixture was cooled to 0° C., 3 equivalents of NaBH₃CN were added, and the solution was stirred for 15 min. An equal volume of 20% HCl in EtOH chilled to 0° C. was subsequently added in a drop-wise fashion over 10 min. The reaction was then allowed to warm to RT and stirred overnight. TLC analysis revealed complete consumption of substrate in all cases. The reaction was neutralized with the addition of Na₂CO₃ until the evolution of gas halted, concentrated under reduced pressure, and CH₂Cl₂ (20 mL) was added. The resulting mixture was washed with saturated Na_{H}CO₃ (2×50 mL), dried over Na₂SO₄, and the collected organics concentrated under reduced pressure. The concentrate was purified by flash chromatography to yield the desired Osubstituted oxyamine product.

### Synthesis of N-methoxybenzylamine (122)

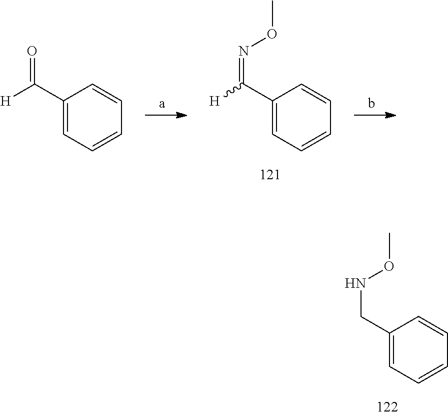

a) MeONH₂.HCl, CH₂Cl₂, pyridine, rt, 2 h; b) NaBH₃CN, 20% HCl in EtOH, 0° C., 18 h.

benzaldehyde-O-methyloxime (121).

According to general procedure 3.4.1.a, benzaldehyde (4.8 g, 49.2 mmol) afforded oxime 121 (6.1 g, 91% crude yield) as a colorless oil. TLC Rf=0.82 (EtOAc/hexanes, 1:8); 1H NMR (400 MHz, CDCl₃) δ 8.03 (s, 1H, NCH), 7.58-7.51 (m, 2H, Ph), 7.37-7.32 (m, 3H, Ph), 3.95 (s, 3H, OCH₃); 13C NMR (100 MHz, CDCl3) δ 148.8, 132.5, 130.0, 129.0, 127.3, 62.2; HRMS-ESI (m/z): [M]⁺. calcd for C8H9NO, 135.0679; found 135.0684.

N-methoxybenzylamine (122).

According to general procedure 3.4.1.b, oxime 121 (6.1 g, 44.9 mmol) provided desired product 122 (3.3 g, 53% yield) as a colorless oil. TLC Rf=0.31 (EtOAc:hexanes, 1:8); 1H NMR (400 MHz, CDCl₃) δ 7.38-7.22 (m, 5H, Ph), 5.71 (br s, 1H, NH), 4.04 (s, 2 H, CH₂NH), 3.50 (d, J=0.4 Hz, 3H, OCH₃); 13C NMR (100 MHz, CDCl₃) δ 137.9, 129.1, 128.7, 127.7, 62.1, 56.5; HRMS-ESI (m/z): [M+H]+ calcd for C₈H₁₂NO, 138.0914; found 138.0916.

### Synthesis of N-ethoxybenzylamine (124)

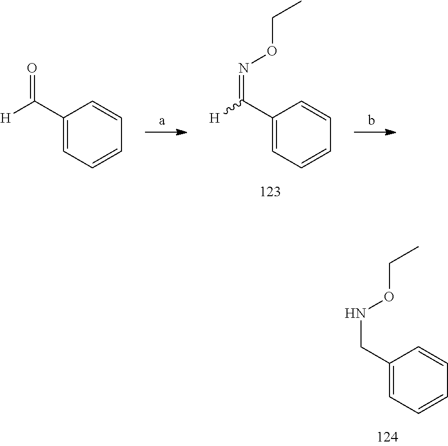

a) EtONH₂.HCl, CH₂Cl₂, pyridine, rt, 2 h; b) NaBH₃CN, 20% HCl in EtOH, 0° C., 18 h.

benzaldehyde-O-ethyloxime (123).

According to general procedure 3.4.1.a, benzaldehyde (1.0 g, 9.4 mmol) afforded oxime 123 (1.32 g, 94% crude yield) as a colorless oil. TLC Rf=0.52, 0.62 (EtOAc/hexanes, 1:8); 1H NMR (400 MHz, CDCl₃): δ 8.07 (s, 1H, NCH), 7.62-7.53 (m, 2H, Ph), 7.42-7.23 (m, 3H, Ph), 4.23 (q, 3J=7.2 Hz, 2H, OCH₂), 1.32 (t, 3J=7.0 Hz, 3H, OCH₂CH₃); 13C NMR (100 MHz, CDCl₃): δ 148.6, 132.8, 130.0, 129.0, 127.3, 70.1, 14.9; HRMS-ESI (m/z): [M+H]⁺ calcd for C₉H₁₂NO, 150.0914; found 150.0919.

N-ethoxybenzylamine (124).

According to general procedure, oxime 123 (1.32 g, 8.8 mmol) provided desired product 124 (0.886 g, 66% yield) as a colorless oil. TLC Rf=0.37 (EtOAc/hexanes, 1:8); 1H NMR (400 MHz, CDCl₃): δ 7.37-7.22 (m, 5H, Ph), 5.59 (br s, 1H, NH), 4.03 (s, 2 H, NHCH₂), 3.69 (q, 3J=7.0 Hz, 2H, OCH₂), 1.13 (t, 3J=7.0 Hz, 3H, OCH₂CH₃); 13C NMR (100 MHz, CDCl₃): δ 137.9, 129.2, 128.6, 127.7, 69.5, 56.9, 14.5; HRMS-ESI (m/z): [M+H]+ calcd for C₉H₁₄NO, 152.1070; found 152.1066.

### Synthesis of N-benzoxybenzylamine (126)

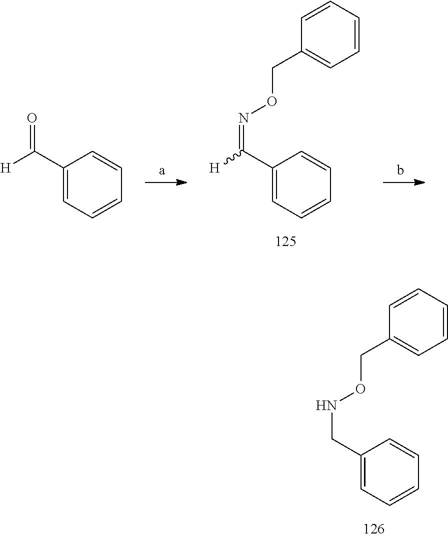 [SMILES: O=Cc1ccccc1 ; C(=NOCc1ccccc1)c1ccccc1 ; c1ccc(CNOCc2ccccc2)cc1]

a) BnONH₂.HCl, CH₂Cl₂, pyridine, rt, 2 h; b) NaBH₃CN, 20% HCl in EtOH, 0° C., 18 h.

benzaldehyde-O-benzyloxime (125).

According to general procedure, benzaldehyde (1.5 g, 14.1 mmol) afforded oxime 125 (2.51 g, 84% crude yield) as a colorless oil. TLC Rf=0.47, 0.56 (EtOAc/hexanes, 1:8); 1H NMR (400 MHz, CDCl₃): δ 8.13 (d, JNCH, Ph=0.8 Hz, 1H, NCH), 7.63-7.52 (m, 2 H, Ph), 7.46-7.25 (m, 8H, Ph), 5.21 (s, 2H, OCH₂); 13C NMR (100 MHz, CDCl₃): δ 149.4, 137.8, 132.6, 130.2, 129.0, 128.8, 128.7, 128.3, 127.4, 76.7; HRMS-ESI (m/z): [M+H]+ calcd for C₁₄H₁₄NO, 212.1070; found 212.1073.

N-benzoxybenzylamine (126).

According to general procedure 3.4.1.b, oxime 125 (2.45 g, 11.6 mmol) provided desired product 126 (0.60 g, 24% yield) as a colorless oil. TLC Rf=0.25 (EtOAc/hexanes, 1:8); 1H NMR (400 MHz, CDCl₃): δ 7.37-7.22 (m, 10H, Ph), 5.71 (br s, 1H, NH), 4.65 (t, JOCH₂, Ph=1.4 Hz, 2H, OCH₂), 4.04 (d, JNHCH₂, Ph=0.4 Hz, 2H, NHCH₂); 13C NMR (100 MHz, CDCl₃): δ 138.2, 137.9, 129.3, 128.8, 128.7, 128.7, 128.1, 127.7, 76.6, 56.8; HRMS-ESI (m/z): [M+H]+ calcd for C₁₄H₁₆NO, 214.1227; found 214.1219.

### Synthesis of N-methoxy-1-naphthalenemethanamine (128)

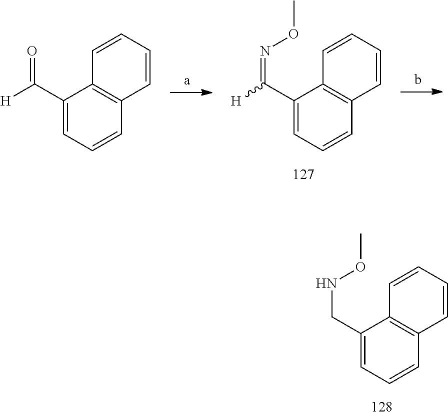

a) MeONH₂.HCl, CH₂Cl₂, pyridine, rt, 2 h; b) NaBH3CN, 20% HCl in EtOH, 0° C., 18 h.

1-naphthaldehyde-O-methyloxime (127).

According to general procedure, 1-naphthaldehyde (2.0 g, 12.7 mmol) gave oxime 127 (2.0 g, 83% crude yield) as a yellow oil. TLC Rf=0.56, 0.65 (EtOAc/hexanes, 1:4); 1H NMR (400 MHz, CDCl₃) δ 8.71 (s, 1H, NCH), 8.52 (m, 1H, Ph), 7.84 (m, 2 H, Ph), 7.74 (m, 1H, Ph), 7.58-7.42 (m, 3H, Ph), 4.06 (s, 3H, OCH3); 13C NMR (100 MHz, CDCl₃) δ 148.5, 133.9, 130.8, 130.5, 128.8, 128.1, 127.4, 127.1, 126.2, 125.4, 124.6, 62.2; HRMS-ESI (m/z): [M]⁺. calcd for C₁₂H₁₁NO, 185.0836; found 185.0842.

N-methoxy-1-naphthalenemethanamine (128).

According to general procedure, oxime 127 (2.0 gm, 10.5 mmol) yielded 128 (1.2 g, 63% yield) as a yellow oil. TLC Rf=0.41 (EtOAc/hexanes, 1:4); 1H NMR (400 MHz, CDCl₃) δ 8.15 (d, J_{Ph, Ph}=8.8 Hz, 1H, Ph), 7.84 (d, J_{Ph, Ph}=8.4 Hz, 1H, Ph), 7.77 (d, J_{Ph, Ph}=8.4 Hz, 1H, Ph), 7.56-7.36 (m, 4H, Ph), 5.77 (br s, 1H, NH), 4.51 (s, 2H, NHCH₂), 3.53 (d, J=0.4 Hz, 3H, OCH₃); 13C NMR (100 MHz, CDCl₃) δ 134.1, 133.0, 132.3, 129.0, 128.7, 127.8, 126.5, 126.0, 125.7, 124.0, 62.1, 54.1; HRMS-ESI (m/z): [M+H]⁺ calcd for C12H14NO, 188.1070; found 188.1070.

### Synthesis of N-ethoxy-1-naphthalenemethanamine (130)

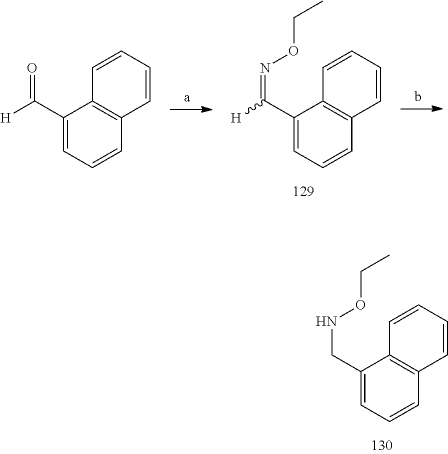

a) EtONH2.HCl, CH₂Cl₂, pyridine, rt, 2 h; b) NaBH₃CN, 20% HCl in EtOH, 0° C., 18 h.

1-naphthaldehyde-O-ethyloxime (129).

According to general procedure, 1-naphthaldehyde (0.5 g, 3.2 mmol) gave oxime 129 (0.57 g, 90% crude yield) as a yellow oil. TLC Rf=0.60, 0.65 (EtOAc/hexanes, 1:8); 1H NMR (400 MHz, CDCl₃): δ 8.77-8.74 (m, 1H, NCH), 8.58 (d, J_{Ph, Ph}=8.3 Hz, 1 H, Ph), 7.90-7.86 (m, 2H, Ph), 7.79 (d, J_{Ph, Ph}=7.2 Hz, 1H, Ph), 7.62-7.46 (m, 3H, Ph), 4.40-4.32 (m, 2H, OCH₂), 1.45-1.39 (m, 3H, OCH₂CH₃); 13C NMR (100 MHz, CDCl₃): δ 148.3, 134.0, 130.8, 130.4, 128.8, 128.4, 127.3, 127.1, 126.2, 125.4, 124.7, 70.0, 14.9; HRMS-ESI (m/z): [M]⁺. calcd for C₁₃H₁₃NO, 199.0992, found 199.0993.

N-ethoxy-1-naphthalenemethanamine (130).

According to general procedure, oxime 129 (0.45 gm, 2.2 mmol) yielded 130 (0.28 g, 62% yield) as a slightly yellow oil. TLC Rf=0.38 (EtOAc/hexanes, 1:8); 1H NMR (400 MHz, CDCl₃) δ 8.17 (d, J_{Ph, Ph}=8.0 Hz, 1H, Ph), 7.84 (dd, J_{Ph, Ph}=0.8, J_{Ph, Ph}=8.0 Hz, 1H, Ph), 7.78 (d, J_{Ph, Ph}=8.0 Hz, 1H, Ph), 7.56-7.36 (m, 4H, Ph), 5.65 (br s, 1H, NH), 4.51 (s, 2H, NHCH₂) 3.73 (q, 3J=7.0 Hz, 2H, OCH₂), 1.14 (t, 3J=7.0 Hz, 3H, OCH₂CH₃); 13C NMR (100 MHz, CDCl₃): δ 134.1, 133.2, 132.4, 129.0, 128.7, 127.9, 126.5, 126.0, 125.7, 124.1, 69.6, 54.5, 14.6; HRMS-ESI (m/z): [M+H]+ calcd for C₁₃H₁₆NO, 202.1227; found 202.1217.

### Synthesis of N-benzoxy-1-naphthalenemethanamine (132)

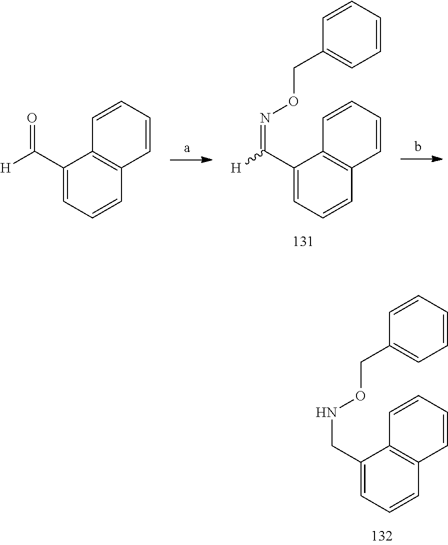 [SMILES: O=Cc1cccc2ccccc12 ; C(=NOCc1ccccc1)c1cccc2ccccc12 ; c1ccc(CONCc2cccc3ccccc23)cc1]

a) BnONH₂.HCl, CH₂Cl₂, pyridine, rt, 2 h; b) NaBH₃CN, 20% HCl in EtOH, 0° C., 18 h.

1-naphthaldehyde-O-benzyloxime (131).

According to general procedure, 1-naphthaldehyde (0.5 g, 3.2 mmol) gave oxime 131 (0.64 g, 77% crude yield) as a yellow oil. TLC Rf=0.54, 0.61 (EtOAc/hexanes, 1:8); 1H NMR (400 MHz, CDCl₃): δ 8.77 (s, 1H, NCH), 8.52 (dd, J_{Ph, Ph}=0.7 Hz, J_{Ph, Ph}=8.3 Hz, 1H, Ph), 7.84 (dd, J_{Ph, Ph}=0.6 Hz, J_{Ph, Ph}=8.2 Hz, 2H, Ph), 7.73 (d, J_{Ph, Ph}=7.1 Hz, 1H, Ph), 7.56-7.29 (m, 8H, Ph), 5.30 (s, 2H, OCH₂); 13C NMR (100 MHz, CDCl₃): δ 149.2, 137.8, 134.1, 131.0, 130.7, 129.0, 128.8, 128.8, 128.3, 128.3, 127.8, 127.3, 126.4, 125.5, 124.9, 76.8; HRMS-ESI (m/z): [M+Na] calcd for C₁₈H₁₅NaNO, 284.1046; found 284.1053.

N-benzoxy-1-naphthalenemethanamine (132).

According to general procedure 3.4.1.b, oxime 131 (0.52 gm, 2.0 mmol) yielded 132 (0.29 g, 55% yield) as a slightly yellow oil. TLC Rf=0.40 (1:8::EtOAc:hexanes); 1H NMR (400 MHz, CDCl₃) δ 8.04-7.98 (m, 1H, Ph), 7.86-7.72 (m, 2H, Ph), 7.56-7.14 (m, 9H, Ph), 5.75 (br s, 1H, NH), 4.66 (s, 2H, OCH₂), 4.48 (s, 2H, NHCH₂); 13C NMR (100 MHz, CDCl₃): δ 138.3, 134.1, 133.0, 132.4, 128.94, 128.89, 128.7, 128.6, 128.1, 128.0, 126.4, 126.0, 125.6, 124.3, 76.6, 54.6; HRMS-ESI (m/z): [M+H]+ calcd for C₁₈H₁₈NO, 264.1283; found 264.1389.

### Synthesis of N-methoxy-2-naphthalenemethanamine (134)

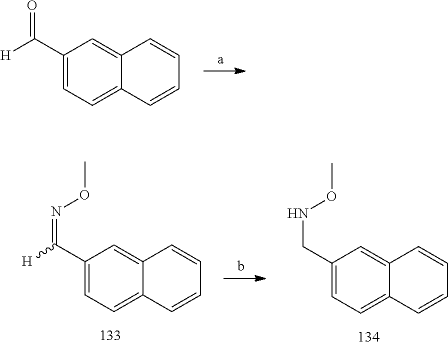

a) MeONH₂.HCl, CH₂Cl₂, pyridine, rt, 2 h; b) NaBH₃CN, 20% HCl in EtOH, 0° C., 18 h.

2-naphthaldehyde-O-methyloxime (133).

According to general procedure, 2-naphthaldehyde (2.0 g, 12.8 mmol) provided the desired oxime 133 (2.3 g, 99% crude yield) as a white solid. TLC Rf=0.48, 0.60 (EtOAc/hexanes, 1:8); 1H NMR (400 MHz, CDCl₃) δ 8.24 (s, 1H, NCH), 7.92-7.80 (m, 5H, Ph), 7.56-7.48 (m) 2H, Ph), 4.058 (s, 3H, OCH₃), 4.055 (s, 3H, OCH₃); 13C NMR (100 MHz, CDCl₃) δ 148.0, 134.4, 133.5, 130.2, 128.9, 128.6, 128.6, 128.2, 127.2, 126.9, 123.3, 62.4; HRMS-ESI (m/z): [M+H]+ calcd for C₁₂H₁₂NO, 186.0841; found 186.0920.

N-methoxy-2-naphthalenemethanamine (134).

According to general procedure, oxime 133 (2.3 g, 12.4 mmol) yielded 134 (1.5 g, 63% yield) as an orange oil. TLC Rf=0.36 (EtOAc/hexanes, 1:4); 1H NMR (400 MHz, CDCl₃) δ 7.84-7.74 (m, 4H, Ph), 7.52-7.39 (m, 3H, Ph), 5.80 (br s, 1H, NH), 4.18 (s, 2H, NHCH₂), 3:50 (t, J=0.4 Hz, 3H, OCH₃); 13C NMR (100 MHz, CDCl₃) δ 135.5, 133.7, 133.1, 128.4, 128.1, 128.0, 127.9, 127.2, 126.3, 126.1, 62.2, 56.6; HRMS-ESI (m/z): [M+H]+ calcd for C₁₂H₁₄NO, 188.1070; found 188.1069.

### Synthesis of N-ethoxy-2-naphthalenemethanamine (136)

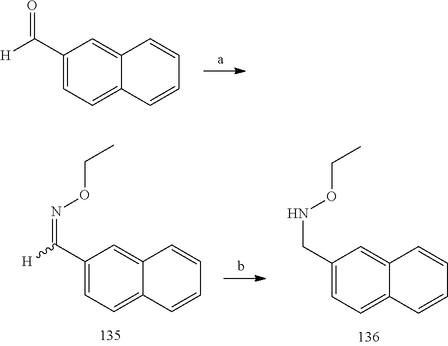

a) EtONH₂.HCl, CH₂Cl₂, pyridine, rt, 2 h; b) NaBH3CN, 20% HCl in EtOH, 0° C., 18 h.

2-naphthaldehyde-O-ethyloxime (135). According to general procedure, 2-naphthaldehyde (2.0 g, 12.8 mmol) provided the desired oxime 135 (2.46 g, 85% crude yield) as an off-white solid. TLC Rf=0.50, 0.60 (EtOAc/hexanes, 1:8); 1H NMR (400 MHz, CDCl₃): δ 8.21 (s, 1H, NCH), 7.89-7.75 (m, 5H, Ph), 7.52-7.42 (m, 2H, Ph), 4.272 (q, 3J=7.0 Hz, 2H, OCH₂), 4.269 (q, 3J=7.0 Hz, 2H, OCH₂), 1.355 (t, 3J=7.0 Hz, 3H, OCH₂CH₃), 1.353 (t, 3J=7.0 Hz, 3H, OCH₂CH₃); 13C NMR (100 MHz, CDCl₃): δ 148.8, 134.4, 135.5, 130.5, 128.8, 128.6, 128.5, 128.2, 127.1, 126.8, 123.3, 70.2, 15.0; HRMS-ESI (m/z): [M+H]+ calcd for C₃₃H₃₄NO, 200.1075; found 200.1080.

N-ethoxy-2-naphthalenemethanamine (136).

According to general procedure, oxime 135 (2.46 g, 12.4 mmol) yielded 136 (1.52 g, 59% yield) as a yellow oil. TLC Rf=0.43 (EtOAc/hexanes, 1:8); 1H NMR (400 MHz, CDCl₃): δ 7.84-7.75 (m, 4H, Ph), 7.52-7.40 (m, 3H, Ph), 5.66 (br s, 1H, NH), 4.19 (s, 2H, NHCH₂), 3.70 (q, 3/=6.8 Hz, 2H, OCH₂), 1.12 (t, 3J=6.8 Hz, 3H, OCH2CH3); 13C NMR (100 MHz, CDCl3): δ 135.5, 133.7, 133.1, 128.3, 128.1, 128.0, 127.4, 129.3, 126.1, 69.7, 57.1, 14.5; HRMS-ESI (m/z): [M+H]+ calcd for C₁₃H₁₆NO, 202.1227; found 202.1222.

### Synthesis of N-benzoxy-2-naphthalenemethanamine (138)

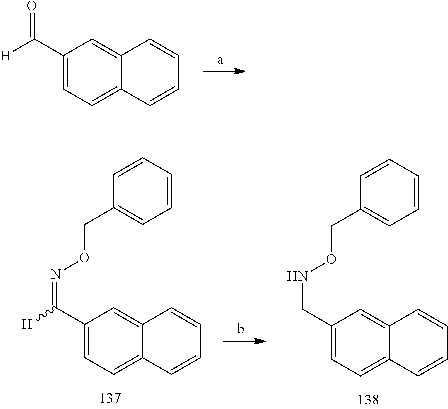 [SMILES: O=Cc1ccc2ccccc2c1 ; C(=NOCc1ccccc1)c1ccc2ccccc2c1 ; c1ccc(CONCc2ccc3ccccc3c2)cc1]

a) BnONH₂.HCl, CH₂Cl₂, pyridine, rt, 2 h; b) NaBH₃CN, 20% HCl in EtOH, 0° C., 18 h.

2-naphthaldehyde-O-benzyloxime (137).

According to general procedure, 2-naphthaldehyde (2.0 g, 12.8 mmol) provided of the desired oxime 137 (3.48 g, 87% crude yield) as an off-white solid. TLC Rf=0.61, 0.74 (EtOAc/hexanes, 1:8); 1H NMR (400 MHz, CDCl₃): δ 8.29 (s, 1H, NCH), 7.89-7.76 (m, 5H, Ph), 7.54-7.43 (m, 4H, Ph), 7.42-7.29 (m, 3H, Ph), 5.26 (s, 2H, OCH₂); 13C NMR (100 MHz, CDCl₃): δ 149.5, 137.8, 134.5, 133.5, 130.3, 128.9, 128.8, 128.8, 128.8, 128.6, 128.3, 128.2, 127.2, 126.9, 123.4, 76.9; HRMS-ESI (m/z): [M+H]+ calcd for C₁₈H₁₆NO, 262.1227; found 262.1234.

N-benzoxy-2-naphthalenemethanamine (138).

According to general procedure, oxime 137 (3.48 g, 13.3 mmol) yielded 138 (0.58 g, 17% yield) as a yellow solid. TLC Rf=0.51 (EtOAc/hexanes, 1:8); 1H NMR (400 MHz, CDCl3): δ 7.87-7.72 (m, 4H, Ph), 7.52-7.40 (m, 3H, Ph), 7.35-7.21 (m, 5H, Ph), 5.81 (br s, 1H, NH), 4.65 (dd, JOCH₂, Ph=2.2 Hz, J=3.0 Hz, 2H, OCH₂), 4.18 (br s, 2H, NHCH2); 13C NMR (100 MHz, CDCl3): δ 138.2, 135.5, 133.7, 133.2, 128.8, 128.8, 128.6, 128.3, 128.1, 128.0, 128.0, 127.4, 126.3, 126.1, 76.7, 57.0; HRMS-ESI (m/z): [M+H]+ calcd for C₁₈H₃₈NO, 264.1383; found 264.1393.

### Synthesis of N-methoxybenzhydrylamine (140)

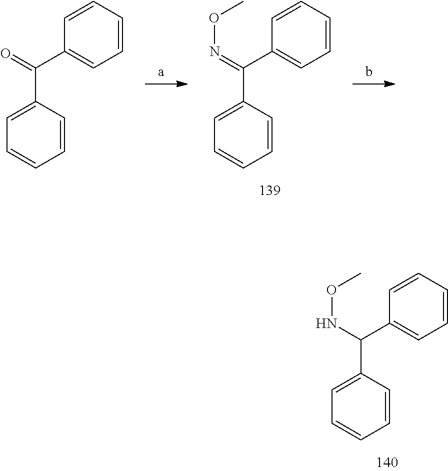 [SMILES: O=C(c1ccccc1)c1ccccc1 ; CON=C(c1ccccc1)c1ccccc1 ; CONC(c1ccccc1)c1ccccc1]

a) MeONH₂.HCl, CH₂Cl₂, pyridine, rt, 2 h; b) NaBH₃CN, 20% HCl in EtOH, 0° C., 18 h.

benzophenone-O-methyloxime (139).

According to general procedure, benzophenone (0.36 g, 2.0 mmol) with 25 equiv. of pyridine provided the desired oxime 139 (0.33 g, 78% crude yield) as a white solid. TLC Rf=0.71 (EtOAc/hexanes, 1:8); 1H NMR (400 MHz, CDCl3): δ 7.56-7.32 (m, 10H, Ph), 4.02-4.01 (m, 3H, OCH₃); 13C NMR (100 MHz, CDCl₃): δ 157.0, 136.7, 133.6, 129.6, 129.5, 129.1, 128.6, 128.4, 128.2, 62.7; HRMS-ESI (m/z): [M+H]+ calcd for C₁₄H₁₄NO, 212.1070; found 212.1079.

N-methoxybenzhydrylamine (140).

According to general procedure, oxime 139 (0.32 g, 1.49 mmol) with 6 equivalents of NaBH3CN yielded 140 (0.17 g, 54% yield) as an oil. TLC Rf=0.43 (EtOAc/hexanes, 1:8); 1H NMR (400 MHz, CDCl₃) δ 7.40-7.16 (m, 10H, Ph), 5.84 (br s, 1H, NH), 5.20 (s, 1 H, NHCH), 3.48 (s, 3H, OCH₃); 13C NMR (100 MHz, CDCl₃) δ 141.4, 128.7, 127.9, 127.7, 69.6, 62.62; HRMS-ESI (m/z): [M+Na]+ calcd for C₁₄H₁₅NaNO, 236.1046; found 236.1057.

### Synthesis of N-ethoxybenzhydrylamine (142)

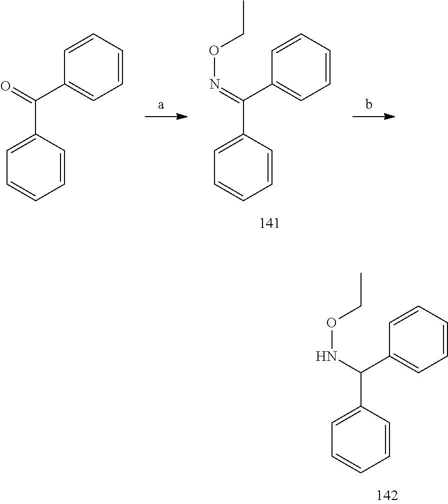 [SMILES: O=C(c1ccccc1)c1ccccc1 ; CCON=C(c1ccccc1)c1ccccc1 ; CCONC(c1ccccc1)c1ccccc1]

a) EtONH₂.HCl, CH₂Cl₂, pyridine, rt, 2 h; b) NaBH3CN, 20% HCl in EtOH, 0° C., 18h.

benzophenone-O-ethyloxime (141).

According to general procedure, benzophenone (0.36 g, 2.0 mmol) with 25 equiv. of pyridine provided the desired oxime 141 (0.41 g, 91% crude yield) as a colorless oil. TLC Rf=0.68 (EtOAc/hexanes, 1:8); 1H NMR (400 MHz, CDCl₃): δ 7.52-7.26 (m, 10H, Ph), 4.24 (q, 3J=7.0 Hz, 2H, OCH₂), 1.30 (t, 3J=7.0 Hz, 3H, OCH₂CH₃); 13C NMR (100 MHz, CDCl₃): δ 156.6, 137.1, 133.8, 129.7, 129.4, 129.0, 128.5, 128.3, 128.2, 70.4, 15.1; HRMS-ESI (m/z): [M+H]+ calcd for C₁₅H₁₆NO, 226.1227; found 226.1235.

N-ethoxybenzhydrylamine (142).

According to general procedure, oxime 141 (0.32 g, 1.49 mmol) with 6 equivalents of NaBH₃CN yielded 142 (0.17 g, 54% yield) as an oil. TLC Rf=0.50 (EtOAc/hexanes, 1:8); 1H NMR (400 MHz, CDCl₃): δ 7.44-7.17 (m, 10H, Ph), 5.78 (br s, 1H, NH), 5.21 (s, 1 H, NHCH), 3.70 (q, 3J=7.0 Hz, 2H, OCH₂CH₃), 1.07 (t, 3J=7.0 Hz, OCH₂CH₃); 13C NMR (100 MHz, CDCl₃): δ 141.6, 128.7, 128.1, 127.7, 70.0, 69.7, 14.43; HRMS-ESI (m/z): [M+Na]+ calcd for C₁₅H₁₇NaNO, 250.1203; found 250.1207.

### Synthesis of N-benzoxybenzhydrylamine (144)

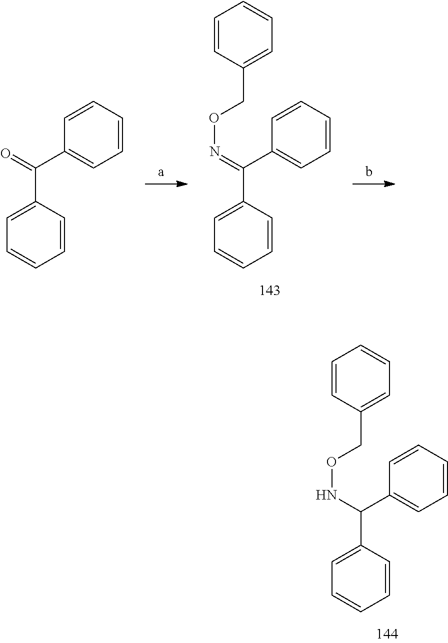 [SMILES: O=C(c1ccccc1)c1ccccc1 ; c1ccc(CON=C(c2ccccc2)c2ccccc2)cc1 ; c1ccc(CONC(c2ccccc2)c2ccccc2)cc1]

a) BnONH₂.HCl, CH₂Cl₂, pyridine, rt, 2 h; b) NaBH₃CN, 20% HCl in EtOH, 0° C., 18 h.

benzophenone-O-benzyloxime (143).

According to general procedure, benzophenone (0.36 g, 2.0 mmol) with 20 equiv. of pyridine provided the desired oxime 143 (0.55 g, 96% crude yield) as a white solid. TLC Rf=0.53 (EtOAc/hexanes, 1:8); 1H NMR (400 MHz, CDCl₃): δ 7.52-7.18 (m, 15H, Ph), 5.23 (s, 2H, OCH₂); HRMS-ESI (m/z): [M+H]+ calcd for C20H18NO, 288.1388; found 288.1398.

N-benzoxybenzhydrylamine (144).

According to general procedure, oxime 143 (0.52 g, 1.8 mmol) with 6 equivalents of NaBH₃CN yielded 144 (0.086 g, 17% yield) as a colorless oil. TLC Rf=0.51 (1:8 EtOAc:hexanes); 1H NMR (400 MHz, CDCl₃): δ 7.46-7.16 (m, 15H, Ph), 5.87 (br s, 1H, NH), 5.24 (br s, 1H, NHCH), 4.65 (s, 2H, OCH2); 13C NMR (100 MHz, CDCl3): δ 141.4, 138.0, 128.9, 128.8, 128.6, 128.1, 128.1, 127.8, 77.0, 69.8; HRMS-ESI (m/z): [M+H]+ calcd for C₂₀H₂₀NO, 290.1540; found 290.1543.

N,N-disubstituted-β-D-glucopyranosylamines (21-32).

General neoglycosylation Procedure.

According to a modified procedure from Goff et al., Osubstituted oxyamine was dissolved in MeOH to a final concentration of 100 mM. 3 equivalents of D-glucose and 1.5 equivalents of acetic acid were added (unless otherwise noted). Reactions were allowed to proceed with stirring at 40° C. and monitored by TLC. Compounds were purified utilizing Extract-Clean SPE SI columns (from Alltech) pre-equilibrated with 1% MeOH in CH₂Cl₂. A gradient of 4 column volumes of 1% MeOH in CH₂Cl₂, 8 column volumes with 5% MeOH in CH₂Cl₂, and 12 column volumes of 10% MeOH in CH₂Cl₂ was sufficient for all purifications. Desired fractions were concentrated with reduced pressure to yield the final β-Dglucoside product.

### Synthesis of N—(N-benzyl-N-methoxy)-β-D-glucopyranosylamine (21)

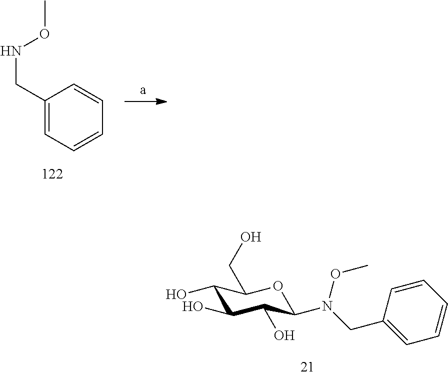

### a) D-glucose, AcOH, MeOH, 40° C., 36 h.

N—(N-benzyl-N-methoxy)-β-D-glucopyranosylamine (21).

According to general procedure, a reaction time of 36 hours with 122 (76 mg, 0.55 mmol) yielded 21 (90 mg, 54% yield) as a colorless syrup. TLC Rf=0.31 (10% MeOH in CH₂Cl₂); 1H NMR (400 MHz, CD₃OD) δ 7.38-7.31 (m, 2H, Ph), 7.25-7.13 (m, 3H, Ph), 4.08 (d, 2J=12.8 Hz, 1H, NCH2), 3.95 (d, 2J=12.8 Hz, 1H, NCH₂), 3.85 (d, J_{H1, H2}=9.2 Hz, 1H, H-1), 3.79 (dd, J_{H5, H6a}=1.6 Hz, J_{H6a, H6b}=12.2 Hz, 1H, H-6a), 3.62 (dd, J_{H5, H6b}=5.4 Hz, 1H, H-6b), 3.48-3.40 (m, 1H, H-2), 3.31 (s, 3H, OCH₃), 3.28-3.18 (m, 2H, H-3, H-4), 3.12-3.02 (m, 1H, H-5); 13C NMR (100 MHz, CD₃OD) δ 138.4, 131.0, 129.2, 128.4, 93.2, 79.6, 79.4, 71.5, 71.2, 62.8, 62.5, 57.6; HRMS-ESI (m/z): [M+Na]+ calcd for C₁₄H₂₁NaNO₆, 322.1262; found 322.1268.

### Synthesis of N—(N-benzyl-N-ethoxy)-β-D-glucopyranosylamine (22)

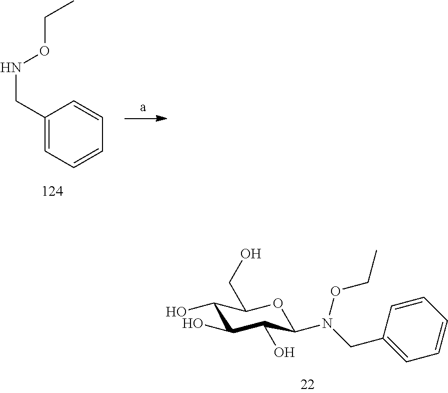

### a) D-glucose, AcOH, MeOH, 40° C., 36 h.

N—(N-benzyl-N-ethoxy)-β-D-glucopyranosylamine (22).

According to general procedure, a reaction time of 36 hours with 124 (73 mg, 0.48 mmol) yielded 22 (114 mg, 75% yield) as a colorless syrup. TLC Rf=0.36 (10% MeOH in CH₂Cl₂); 1H NMR (400 MHz, CD₃OD) δ 7.46-7.38 (m, 2H, Ph), 7.34-7.20 (m, 3H, Ph), 4.15 (d, 2J=12.6 Hz, 1H, NCH₂), 4.04 (d, 2J=12.6 Hz, 1H, NCH₂), 3.94 (d, J_{H1, H2}=8.8 Hz, 1H, H-1), 3.88 (dd, J_{H5, H6a}=2.0 Hz, J_{H6a, H6b}=12.0 Hz, 1H, H-6a), 3.76-3.62 (m, 2H, H-6b, OCH2), 3.60-3.46 (m, 2H, OCH₂, H-2), 3.36-3.28 (m, 2H, H-3, H-4), 3.20-3.12 (m, 1H, H-5), 0.96 (t, 3J=7.2 Hz, 3H, OCH₂CH₃); 13C NMR (100 MHz, CD₃OD) δ 138.5, 131.1, 129.2, 128.4, 93.3, 79.5, 97.4, 71.5, 71.1, 70.8, 62.7, 58.0, 14.0; HRMSESI (m/z): [M+Na]+ calcd for C₁₅H₂₃NaNO₆, 336.1418; found 336.1431.

### Synthesis of N—(N-benzoxy-N-benzyl)-β-D-glucopyranosylamine (23)

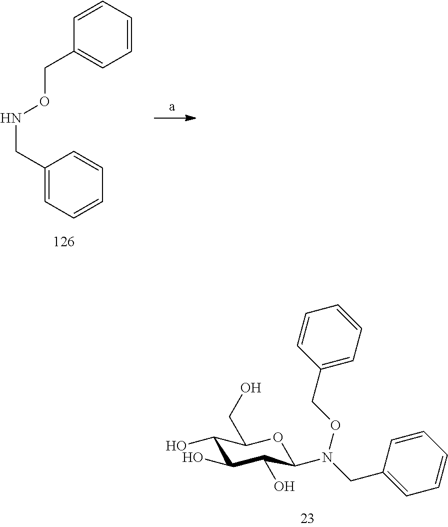

### a) D-glucose, AcOH, MeOH, 40° C., 36 h.

N—(N-benzoxy-N-benzyl)-β-D-glucopyranosylamine (23).

According to general procedure, a reaction time of 36 hours with 126 (92 mg, 0.43 mmol) yielded 23 (123 mg, 76% yield) as a colorless syrup. TLC Rf=0.50 (10% MeOH in CH₂Cl₂); 1H NMR (400 MHz, CD₃OD) δ 7.48-7.40 (m, 2H, Ph), 7.36-7.20 (m, 6H, Ph), 7.16-7.08 (m, 2H, Ph), 4.61 (d, 2J=9.8 Hz, 1H, OCH₂), 4.40 (d, 2J=9.8 Hz, 1H, OCH₂), 4.17 (d, 2J=12.8 Hz, 1H, NCH₂), 4.05 (d, 2J=12.8 Hz, 1H, NCH₂), 4.00 (d, J_{H1, H2}=8.8 Hz, 1H, H-1), 3.84 (dd, J_{H5, H6a}=2.2 Hz, J_{H6a, H6b}=12.2 Hz, 1H, H-6a), 3.72-3.60 (m, 2H, H-6b, H-2), 3.40-3.30 (m, 2H, H-3, H-4), 3.20-3.10 (m, 1H, H-5); 13C NMR (100 MHz, CD₃OD) δ 138.4, 137.5, 131.3, 130.6, 129.3, 129.3, 129.3, 128.5, 93.4, 79.5, 79.5, 78.0, 71.6, 71.0, 62.6, 58.1; HRMS-ESI (m/z): [M+Na]+ calcd for C₂₀H₂₅NaNO₆, 398.1575; found 398.1569.

### Synthesis of N—(N-methoxy-N-naphthalen-1-yl-methyl)-β-D-glucopyranosylamine (24)

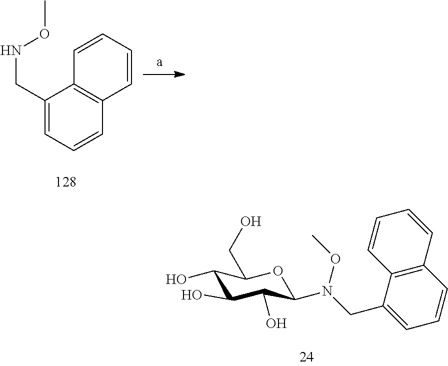

### a) D-glucose, AcOH, MeOH, 40° C., 36 h.

N—(N-methoxy-N-naphthalen-1-yl-methyl)-β-D-glucopyranosylamine (24).

According to general procedure, a reaction time of 36 hours with 128 (82 mg, 0.44 mmol) yielded 24 (113 mg, 74% yield) as a colorless syrup. TLC Rf=0.28 (10% MeOH in CH₂Cl₂); 1H NMR (400 MHz, CD₃OD) δ 8.39 (d, J_{Ph, Ph}=8.4 Hz, 1H, Ph), 7.81 (dd, J_{Ph, Ph}=8.0 Hz, JPh, Ph=15.6 Hz, 2H, Ph), 7.59-7.37 (m, 4H, Ph), 4.64 (d, 2J=12.6 Hz, 1H, NCH₂), 4.53 (d, 2J=12.6 Hz, 1H, NCH₂), 3.97 (dd, J_{H5, H6a}=2.4 Hz, J_{H6a, H6b}=12.2 Hz, 1H, H-6a), 3.96 (d, J_{H1, H2}=8.8 Hz, 1H, H-1), 3.79 (dd, J_{H5, H6b}=5.6 Hz, 1H, H-6b), 3.60 (t, J_{H2, H3}=8.8 Hz, 1 Hz, H-2), 3.38 (s, 3H, OCH₃), 3.42-3.27 (m, 21-1, H-3, H-4), 3.22-3.14 (m, 1H, H-5); 13C NMR (100 MHz, CD₃OD) δ 135.2, 133.9, 133.7, 130.0, 129.5, 129.4, 127.0, 126.6, 126.3, 125.8, 93.0, 79.6, 79.4, 71.5, 71.2, 62.8, 62.5, 55.2; HRMSESI (m/z): [M+Na]+ calcd for C₁₈H₂₃NaNO₆, 372.1418; found 372.1420.

### Synthesis of N—(N-ethoxy-N-naphthalen-1-yl-methyl)-β-D-glucopyranosylamine (25)

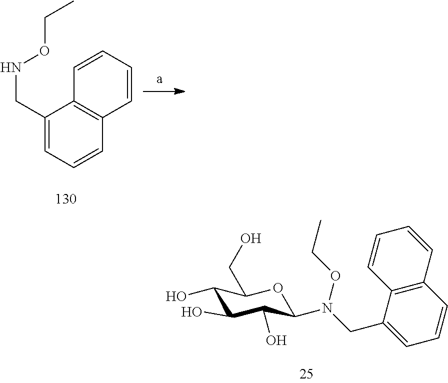

### a) D-glucose, AcOH, MeOH, 40° C., 36 h.

N—(N-ethoxy-N-naphthalen-1-yl-methyl)-O-D-glucopyranosylamine (25). According to general procedure, a reaction time of 36 hours with 130 (77 mg, 0.38 mmol) yielded 25 (71 mg, 52% yield) as a colorless syrup. TLC Rf=0.33 (10% MeOH in CH₂Cl₂); 1H NMR (400 MHz, CD₃OD) δ 8.38 (d, J_{Ph, Ph}=8.4 Hz, 1H, Ph), 7.81 (dd, J_{Ph, Ph}=8.2 Hz, JPh, Ph=14.6 Hz, 2H, Ph), 7.59-7.38 (m, 4H, Ph), 4.62 (d, 2J=12.6 Hz, 1H, NCH₂), 4.53 (d, 2J=12.6 Hz, 1H, NCH₂), 3.97 (d, J_{H1, H2}=8.8 Hz, 1H, H-1), 3.96 (dd, J_{H5, H6a}=2.4 Hz, J_{H6a, H6b}=12.2 Hz, 1H, H-6a), 3.79 (dd, J_{H5, H6b}=5.2 Hz, 1H, H-6b), 3.83-3.75 (m, 1H, OCH₂), 3.58 (t, J=9.0 Hz, 1H, H2), 3.54-3.44 (m, 1H, OCH₂), 3.42-3.27 (m, 2H, H-3, H-4), 3.20-3.13 (m, 1H, H-5), 0.92 (t, 3J=7.0 Hz, 3H, OCH₂CH₃); 13C NMR (100 MHz, CD₃OD) δ 135.2, 134.0, 134.0, 130.0, 129.4, 129.4, 127.0, 126.6, 126.2, 125.8, 93.2, 79.6, 79.5, 71.6, 71.1, 70.7, 62.8, 55.6, 14.1; HRMS-ESI (m/z):

[M+Na]+ calcd for C₁₉H₂₅NaNO₆, 386.1575; found 386.1585.

### Synthesis of N—(N-benzoxy-N-naphthalen-1-yl-methyl)-β-D-glucopyranosylamine (26)

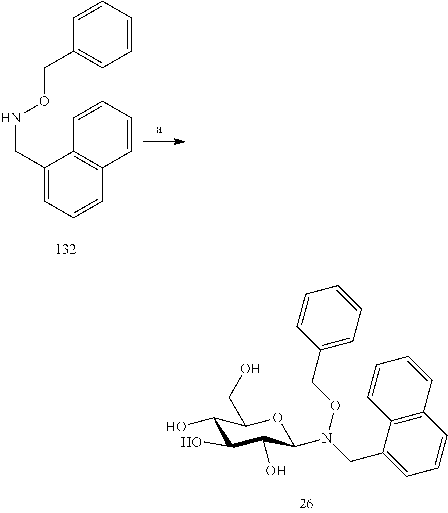

### a) D-glucose, AcOH, MeOH, 40° C., 96 h.

N—(N-benzoxy-N-naphthalen-1-yl-methyl)-β-D-glucopyranosylamine (26).

According to general procedure, a reaction time of 96 hours with 132 (88 mg, 0.33 mmol) yielded 26 (69 mg, 49% yield) as a colorless syrup. TLC Rf=0.40 (10% MeOH in CH₂Cl₂); 1H NMR (400 MHz, CD₃OD) δ 8.36-8.24 (m, 1H, Ph), 7.90-7.77 (m, 2H, Ph), 7.60-7.38 (m, 4H, Ph), 7.30-7.18 (m, 3H, Ph), 7.14-7.04 (m, 2H, Ph), 4.67 (d, 2J=12.4 Hz, 1H, NCH₂), 4.62-4.50 (m, 2H, NCH₂, OCH₂), 4.33 (d, 2J=9.6 Hz, 1H, OCH₂), 4.03 (d, J_{H1, H2}=8.8 Hz, 1H, H-1), 3.98-3.88 (m, 1H, H-6a), 3.80-3.64 (m, 2H, H-2, H-6), 3.42-3.28 (m, 2H, H-3, H-4), 3.23-3.12 (m, 1H, H-5); 13C NMR (100 MHz, CD₃OD) δ 137.7, 135.3, 134.1, 134.0, 130.7, 130.3, 129.6, 129.4, 129.3, 129.3, 127.1, 126.7, 126.3, 126.1, 93.4, 79.65, 79.57, 77.9, 71.7, 71.1, 62.7, 55.9; HRMS-ESI (m/z): [M+Na]+ calcd for C₂₄H₂₇NaNO₆, 448.1731; found 448.1746.

### Synthesis of N—(N-methoxy-N-naphthalen-2-yl-methyl)-β-D-glucopyranosylamine (27)

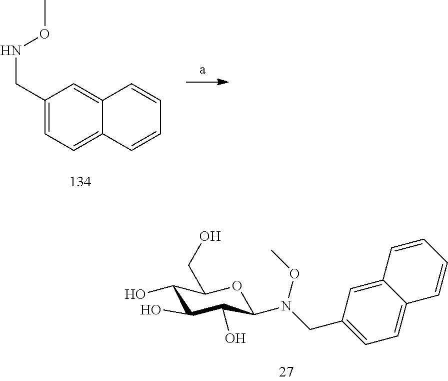

### a) D-glucose, AcOH, MeOH, 40° C., 36 h.

N—(N-methoxy-N-naphthalen-2-yl-methyl)-β-D-glucopyranosylamine (27).

According to general procedure, a reaction time of 36 hours with 134 (78 mg, 0.42 mmol) yielded 27 (49 mg, 33% yield) as a white solid. TLC Rf=0.23 (10% MeOH in CH₂Cl₂); 1H NMR (400 MHz, CD₃OD) δ 7.78-7.68 (m, 4H, Ph), 7.55-7.49 (m, H, Ph), 7.38-7.30 (m, 2H, Ph), 4.23 (d, 2J=12.6 Hz, 1H, NCH₂), 4.11 (d, 2J=12.6 Hz, 1H, NCH₂), 3.88 (d, J_{H1, H2}=9.2 Hz, 1H, H-1), 3.83 (dd, J_{H5, H6a}=2.0 Hz, J_{H6a, H6b}=12.0 Hz, 1H, H-6a), 3.64 (dd, J_{H5, H6b}=5.6 Hz, 1H, H-6b), 3.50-3.42 (m, 1H, H-2), 3.26-3.18 (m, 2H, H-3, H-4), 3.12-3.04 (m, 1 H, H-5); 13C NMR (100 MHz, CD₃OD) δ 136.0, 134.8, 134.4, 134.0, 129.8, 129.0, 128.8, 128.6, 127.0, 126.8, 93.4, 79.8, 79.5, 71.6, 71.3, 62.9, 62.6, 57.8; HRMS-ESI (m/z): [M+Na]+ calcd for C₁₈H₂₃NaNO₆, 372.1418; found 372.1408.

### Synthesis of N—(N-ethoxy-N-naphthalen-2-yl-methyl)-β-D-glucopyranosylamine (28)

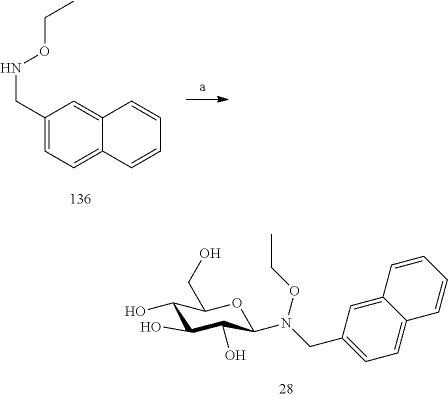

### a) D-glucose, AcOH, MeOH, 40° C., 36 h.

N—(N-ethoxy-N-naphthalen-2-yl-methyl)-β-D-glucopyranosylamine (28).

According to general procedure, a reaction time of 36 hours with 136 (90 mg, 0.45 mmol) yielded 28 (99 mg, 61% yield) as a white solid. TLC Rf=0.30 (10% MeOH in CH₂Cl₂); 1H NMR (400 MHz, CD₃OD) δ 7.84-7.74 (m, 4 H, Ph), 7.55-7.60 (m, 1H, Ph), 7.44-7.36 (m, 2H, Ph), 4.29 (d, 2J=12.8 Hz, 1H, NCH₂), 4.18 (d, 2J=12.8 Hz, 1H, NCH₂), 3.96 (d, J_{H1, H2}=9.2 Hz, 1H, H-1), 3.90 (dd, J_{H5, H6a}=2.0 Hz, J_{H6a, H6b}=12.1 Hz, 1H, H-6a), 3.73 (dd, J_{H5, H6b}=5.4 Hz, 1H, H-6b), 3.70-3.62 (m, 1H, OCH₂), 3.58-3.46 (m, 2H, OCH₂, H-2), 3.36-3.25 (m, 2H, H-3, H-4), 3.20-3.12 (m, 1H, H-5), 0.96-0.88 (m, 3H, OCH₂CH₃); 13C NMR (100 MHz, CD₃OD) δ 136.1, 134.8, 134.3, 129.8, 129.1, 128.76, 128.75, 128.6, 127.0, 126.8, 93.5, 79.6, 79.5, 71.6, 71.2, 70.9, 62.8, 58.1, 14.0; HRMS-ESI (m/z): [M+Na]+ calcd for C₁₉H₂₅NaNO₆, 386.1575; found 386.1579.

### Synthesis of N—(N-benzoxy-N-naphthalen-2-yl-methyl)-β-D-glucopyranosylamine (29)

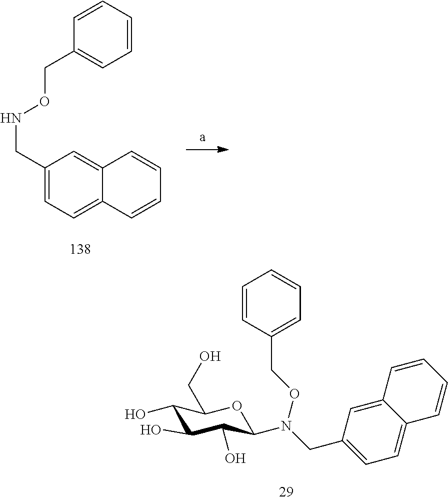

### a) D-glucose, AcOH, MeOH, 40° C., 36 h.

N—(N-benzoxy-N-naphthalen-2-yl-methyl)-β-D-glucopyranosylamine (29).

According to general procedure, a reaction time of 36 hours with 138 (68 mg, 0.26 mmol) yielded 29 (88 mg, 80% yield) as a white solid. TLC Rf=0.40 (10% MeOH in CH₂Cl₂); 1H NMR (400 MHz, DMSO-d6) δ 7.98-7.88 (m, 4H, Ph), 7.66-7.62 (m, 1H, Ph), 7.57-7.49 (m, 2H, Ph), 7.32-7.24 (m, 3H, Ph), 7.18-7.12 (m, 2H, Ph), 4.63 (d, 2J=10.0 Hz, 1H, OCH₂), 4.43 (d, 2J=10.0 Hz, 1H, OCH₂), 4.26 (s, 2H, NCH₂), 3.89 (d, J_{H1, H2}=8.8 Hz, 1H, H-1), 3.82-3.74 (m, 1H, H-6a), 3.56-3.46 (m, 2H, H-2, H-6b), 3.22-3.14 (m, 1H, H-3), 3.10-3.02 (m, 2H, H-4, H-5); 13C NMR (100 MHz, DMSO-d6) δ 146.7, 144.9, 142.8, 142.2, 138.8, 138.4, 138.2, 138.1, 137.8, 137.5, 137.4, 136.0, 135.8, 101.8, 88.5, 87.5, 87.9, 85.6, 80.0, 79.9, 71.2, 66.2, HRMS-ESI (m/z): [M+Na]+ calcd for C₂₄H₂₇NaNO₆, 448.1731; found 448.1726.

### Synthesis of N—(N-benzhydryl-N-methoxy)-β-D-glucopyranosylamine (30)

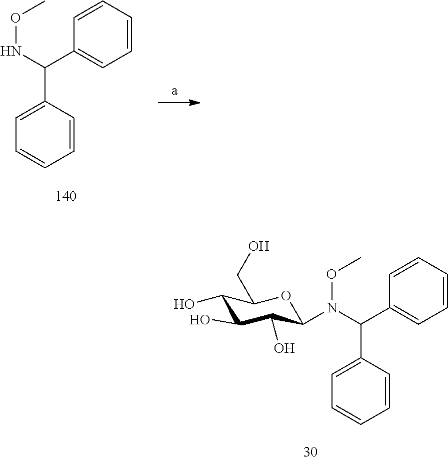

a) D-glucose, AcOH, MeOH, 40° C., 10 days.

N—(N-benzhydryl-N-methoxy)-β-D-glucopyranosylamine (30).

According to general procedure, a reaction time of 10 days with 140 (113 mg, 0.53 mmol) yielded 30 (92 mg, 46% yield) as a yellow oil. TLC Rf=0.32 (10% MeOH in CH₂Cl₂); 1H NMR (400 MHz, CD₃OD) δ 7.65 (d, JPh, Ph=7.6 Hz, 2 H, Ph), 7.55 (d, JPh, Ph=7.6 Hz, 2H, Ph), 7.44-7.16 (m, 6H, Ph), 5.44 (s, 1H, NCH), 3.88-3.83 (m, 1H, H-6a), 3.81 (d, J_{H1, H2}=9.6 Hz, 1H, H-1), 3.73-3.64 (m, 1H, H-6b), 3.63-3.55 (m, 1H, H-2), 3.39 (s, 3H, OCH₃), 3.32-3.25 (m, 1H, H-4), 3.15 (t, J=9.0 Hz, 1H, H-3), 2.76-2.70 (m, 1H, H-5); 13C NMR (100 MHz, CD₃OD) δ 145.5, 142.1, 129.9, 129.7, 129.6, 129.1, 128.6, 128.2, 91.8, 79.74, 79.71, 73.3, 71.7, 71.1, 64.2, 62.8; HRMS-ESI (m/z): [M+Na]+ calcd for C₂₀H₂₅NaNO₆, 398.1575; found 398.1584.

### Synthesis of N—(N-benzhydryl-N-ethoxy)-β-D-glucopyranosylamine (31)

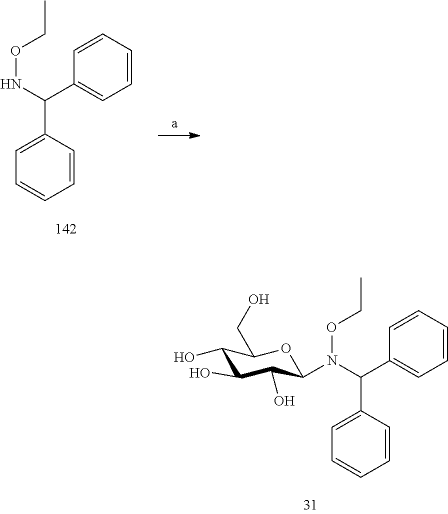

a) D-glucose, AcOH, MeOH, 40° C., 7 days.

N—(N-benzhydryl-N-ethoxy)-β-D-glucopyranosylamine (31).

According to general procedure, a reaction time of 7 days with 142 (29 mg, 0.13 mmol) yielded 31 (15 mg, 30% yield) as a yellow oil. TLC Rf=0.37 (10% MeOH in CH₂Cl₂); 1H NMR (400 MHz, CD₃OD) δ 7.64 (d, J_{Ph, Ph}=7.6 Hz, 2H, Ph), 7.56 (d, J_{Ph, Ph}=7.6 Hz, 2H, Ph), 7.32-7.25 (m, 4H, Ph), 7.24-7.16 (m, 2H, Ph), 5.44 (s, 1H, NCH), 3.92-3.81 (m, 2H, OCH₂, H-1), 3.70 (dd, J_{H5, H6a}=4.6 Hz, J_{H6a, H6b}=12.2 Hz, 1H, H-6a), 3.56 (t, J=8.8 Hz, 1H, H-2), 3.52-3.42 (m, 1H, OCH₂), 3.36-3.27 (m, 1H, H-4, H-6b), 3.16 (t, J=8.8 Hz, 1H, H-3), 2.75-2.69 (m, 1H, H-5), 0.70 (t, 3J=7.2 Hz, 3H, OCH₂CH₃); 13C NMR (100 MHz, CD₃OD) δ 143.9, 142.1, 130.0, 129.6, 129.0, 128.6, 128.5, 128.2, 91.7, 79.7, 79.6, 73.3, 72.4, 71.7, 70.9, 62.6, 13.7; HRMS-ESI (m/z): [M+Na]+ calcd for C₂₁H₂₇NaNO₆,412.1731; found 412.1731.

### Synthesis of N—(N-benzhydryl-N-benzoxy)-β-D-glucopyranosylamine (32)

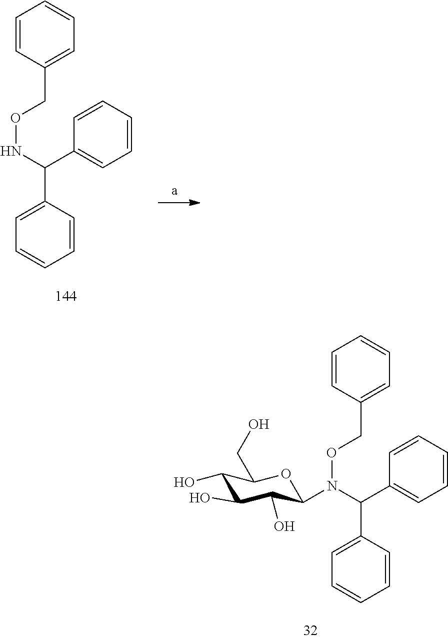

a) D-glucose, AcOH, MeOH, 40° C., 7 days.

N—(N-benzhydryl-N-benzoxy)-β-D-glucopyranosylamine (32).

According to general procedure, a reaction time of 7 days with 144 (115 mg, 0.40 mmol) yielded 32 (57 mg, 32% yield) as a yellow solid. TLC Rf=0.49 (10% MeOH in CH₂Cl₂); 1H NMR (400 MHz, CD₃OD) δ 7.78-7.73 (m, 2H, Ph), 7.65-7.60 (m, 2H, Ph), 7.46-7.10 (m, 9H, Ph), 7.76-7.70 (m, 2H, Ph), 5.49 (s, 1H, NCH), 4.78 (d, 2J=9.0 Hz, 1H, OCH₂), 4.43 (d, 2J=9.0 Hz, 1H, OCH₂), 3.94 (d, J_{H1, H2}=8.8 Hz, 1H, H-1), 3.81 (dd, J_{H5, H6a}=2.2 Hz, J_{H6a, H6b}=12.2 Hz, 1H, H-6a), 3.77 (t, J=8.8 Hz, 1H, H-2), 3.67 (dd, J_{H5, H6b}=2.2 Hz, 1H, H-6b), 3.38-3.29 (m, 1H, H-4), 3.21 (t, J=8.8 Hz, 1H, H-3), 2.79-2.70 (m, 1H, H-5); 13C NMR (100 MHz, CD₃OD) δ 143.4, 142.1, 137.1, 130.6, 130.3, 129.7, 129.6, 129.5, 129.3, 129.2, 129.1, 128.6, 128.5, 91.7, 79.7, 79.5, 79.2, 73.3, 71.7, 70.7, 62.3; HRMS-ESI (m/z): [M+Na]+ calcd for C₂₆H₂₉NaNO₆, 474.1888; found 474.1877.

β-D-glucoside Screening.

Reactions containing 2.1 μM (10 μg) of purified OleD variant, 1 mM of UDP or TDP, and 1 mM of β-D-glucopyranoside (1-32) in Tris-HCl (50 mM, pH 8.5) with a final volume of 100 μl were incubated at room temperature for 1 hour. Samples were frozen in a bath of dry ice and acetone and stored at −20° C. Following, samples were thawed at 4° C. and filtered through a MultiScreen Filter Plate (from Millipore, Billerica, Mass., USA) according to manufacturer's instructions and evaluated for formation of UDP-(33a) or TDP-α-D-glucose (33b) by analytical reverse-phase HPLC with a 250 mm×4.6 mm Gemini-NX 5μ C18 column (Phenomenex, Torrance, Calif., USA) using a linear gradient of 0% to 15% CH₃CN (solvent B) over 15 minutes (solvent A=aqueous 50 mM triethylammonium acetate buffer [from Sigma-Aldrich, St. Louis, Mo., USA], flow rate=1 ml min-1, with detection monitored at 254 nm).

pH Optimization of Reverse Reaction.

All reactions were performed in a final volume of 500 μl dH₂O buffered with 50 mM MES (pH 6.0 or 6.5) 50 mM MOPS (pH 6.5 or 7.0) or 50 mM Tris-HCl (pH 7.0, 7.5, 8.0, 8.5 or 9.0) with 0.21 μM (5 μg) OleD variant TDP-16, 0.05 mM of 2-chloro-4-nitrophenyl-β-D-glucoside (9) as donor and 0.05 mM of UDP as acceptor. Absorbance measurements were taken at t=0 and 60 minutes and Δ410 nm was calculated. Rates of 2-chloro-4-nitrophenolate production were then calculated by comparing them against standard curves at the corresponding pH and buffer. Collectively, the standard deviation in rates from pH 7.0 to pH 8.5 with Tris-HCl as buffer was <5%. Rates dropped sharply outside of this pH range or with change in buffer. These observations are consistent with those previously reported for forward reactions with wild-type OleD.

NDP Screening with 2-chloro-4-nitrophenyl glucoside (9).

All reactions were performed in a final volume of 200 μl Tris-HCl buffer (50 mM, pH 8.5) with 2.1 μM (20 μg) OleD variant TDP-16, 1 mM of 2-chloro-4-nitrophenyl-β-D-glucoside (9) as donor and 1 mM of either UDP, TDP, CDP, ADP or GDP as acceptor. Reactions were allowed to proceed for 5 hours, quenched with an equal volume of 40 mM phosphoric acid (adjusted to pH 6.5 with triethylamine) and heated for 30 seconds on a heat block. Following, samples were centrifuged at 10,000 g for 30 min at 0° C. and the supernatant removed for analysis. The clarified reaction mixtures were analyzed by analytical reverse-phase HPLC. HPLC was conducted with a Supelcosil LC18-T (3 μm, 150×4.6 mm) column (from Sigma-Aldrich St. Louis, Mo., USA) with a gradient of 0% B to 100% B over 20 min (A=40 mM phosphoric acid [adjusted to pH 6.5 with triethylamine]; B=10% MeOH in 40 mM phosphoric acid [adjusted to pH 6.5 with triethylamine]; flow rate=0.5 mL min-1) and detection monitored at 254 nm.

(U/T)DP-α-D-glucose (33a-b) Scale-Up/Characterization—General Reaction Procedure.

Reactions were conducted at 25° C. in 2 mL Tris-HCl (50 mM, pH 8.5) with 2-chloro-4-nitrophenyl β-D-glucopyranoside (9), either UDP or TDP, and 4.2 μM (^{˜}400 μg) of OleD variant P67T/S312F/A242L/Q268V. At 6 hours, 4 mL of 50 mM Tris-HCl (50 mM, pH 7.0) and 5 μL of alkaline phosphatase (100 U, Roche) were added. At 7.5 hours, the reaction was passed through a 10K MWCO filter, frozen at −80° C., and lyophilized. The dried reaction was dissolved in 2 mL of ddH₂O and the desired product purified by semi-preparative HPLC with a Supelcosil LC18, 5 μm, 25 cm×10 mm column (Supelco) using a gradient of 0% to 12.5% CH₃CN (solvent B) over 12.5 min, 12.5% to 90% B over 1 min, 90% B for 5 min (A=50 mM PO4-2, 5 mM tetrabutylammonium bisulfate, 2% acetonitrile, pH adjusted to 6.0 with KOH; flow rate=5 mL min-1; A254 nm). Desired fractions were concentrated under reduced pressure, frozen at −80° C., and lyophilized. The resulting products were dissolved in 2 mL of ddH2O and purified by semi-preparative HPLC with the column mentioned above using linear gradient of 0% B to 10% B over 10 min (A=50 mM triethylammonium acetate buffer; B=acetonitrile; flow rate=5 mL min-1; A254 nm). The desired fractions were concentrated via reduced pressure, frozen at −80° C., and lyophilized multiple times. Products were confirmed by mass spectrometry and via 1H, 13C, and 31P NMR using a Varian UNITYINOVA 500 MHz instrument (Palo Alto, Calif., USA) with a Nalorac qn6121 probe (Martinez, Calif., USA). Assignments were aided with gCOSY and gHSQC methods.

### Synthesis of uridine 5′-diphosphate α-D-glucose (33a)

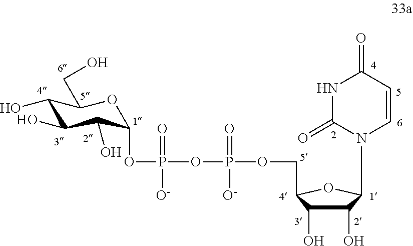

UDP (9.0 mg, 0.022 mmol) and 8.9 mg of 9 (0.025 mmol) in the above method yielded 6.9 mg of 33a (0.012 mmol, 55% isolated yield). 1H NMR (500 MHz, D₂O) δ 7.97 (d, J_{H-6, H-5}=8.1 Hz, 1H, H-6), 6.05-5.90 (m, 2H, H-1′, H-5), 5.61 (dd, J_{H-1″, H-2″}=3.5 Hz, J_{H-1″}, P=7.2 Hz, 1H, H-1″), 4.38 (m, 2H, H-2′, H-3′), 4.31-4.28 (m, 1H, H-4′), 4.27-4.20 (m, 2H, H-5a′, H-5b′), 3.91 (ddd, J_{H-5″, H-6a″}=2.2 Hz, J_{H-5″, H-6b″}=4.3 Hz, J_{H-5″, H-4″}=9.9 Hz, 1H, H-5″), 3.87 (dd, J_{H-6a″, H-5″}=2.2 Hz, J_{H-6a″, H-6b″}=12.5 Hz, 1H, H-6a″), 3.81-3.75 (m, 2H, H-3″, H-6b″), 3.56-3.51 (m, 1H, H-2″), 3.47 (dd, J_{H-4″, H-3″}=9.9 Hz, J_{H-4″, H-5″}=9.9 Hz, 1H, H-4″); 13C NMR (126 MHz, D₂O) δ 167.2 (C-4), 152.7 (C-2), 142.5 (C-6), 103.5 (C-5), 96.4 (J_{C-1″, P}=6.7 Hz, C-1″), 89.2 (C-1′), 84.1 (J_{C-4′, P}=9.2 Hz, C-4′), 74.6 (C-2′), 73.7 (C-5″), 73.6 (C-3″), 72.5 (J_{C2-P″}=8.5 Hz, C-2″), 70.5 (C-3′), 70.0 (C-4′), 65.8 (J_{C-5′, P}=5.5 Hz, C-5′), 61.2 (C-6″); 31P NMR (202 MHz, D₂O) δ −10.0 (d, J_{P, P}=20.7 Hz), −11.7 (d, J_{P, P}=20.7 Hz); HRMS-ESI (m/z): [M+Na]+ calcd for C₁₅H₂₂N₂NaO₁₇P₂ 587.02969; found 587.02954; spectral data are consistent with those reported by Bae et al.

### Synthesis of thymidine 5′-diphosphate α-D-glucose (33b)

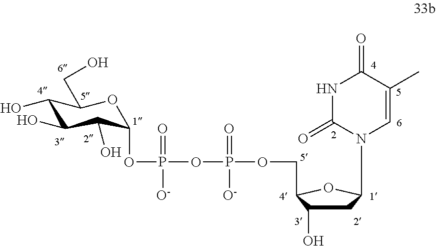

TDP (9.0 mg, 0.022 mmol) and 9.0 mg of 9 (0.025 mmol) in the above method yielded 7.7 mg of 33b (0.013 mmol, 61% isolated yield). 1H NMR (500 MHz, D₂O) δ 7.75 (d, J_{H-6, 5-CH3}=1.0 Hz, 1H, H-6), 6.36 (t, J_{H-1′, H-2′s}=7.0 Hz, 1H, H-1′), 5.61 (dd, J_{H-1″, H-2″}=3.5 Hz, J_{H-1″, P}=7.2 Hz, 1H, H-1″), 4.65-4.62 (m, 1H, H-3′), 4.20-4.17 (m, 3H, H-4′, H-5a′, H-5b′), 3.91 (ddd, J_{H5″, H-6a″}=2.3 Hz, J_{H5″, H-6b″}=4.4 Hz, J_{H-5″, H-4″}=9.8 Hz, 1H, H-5″), 3.87 (dd, J_{H-6a, H-5″}=2.3 Hz, J_{H-6a″, H-6b″}=12.4 Hz, 1H, H-6a″), 3.81-3.75 (m, 2H, H-3″, H-6b″), 3.56-3.50 (m, 1H, H-2″), 3.47 (dd, J_{H-4″, H-3″}=9.8 Hz, J_{H-4″, H-5″}=9.8 Hz, 1H, H-4″), 2.41-2.35 (m, 2 H, H-2a′, H-2b′), 1.94 (d, J_{5-CH3, H-6}=1.0 Hz, 1H, 5-CH3); 13C NMR (126 MHz, D₂O) δ 167.2 (C-4), 152.4 (C-2), 138.0 (C-6), 112.4 (C-5), 96.3 (J_{C-1″, P}=6.7 Hz, C-1″), 86.1 (J_{C-4′, P}=9.1 Hz, C-4′), 85.7 (C-1′), 73.6 (C-3″), 73.5 (C-5″), 72.4 (J_{C-2″, P}=8.6 Hz, C-2″), 71.8 (C-3′), 69.9 (C-4″), 66.2 (J_{C-5′, P}=5.7 Hz, C-5′), 61.1 (C-6″), 39.4 (C-2′), 12.5 (5-CH₃); 31P NMR (202 MHz, D₂O) δ −10.18 (d, J_{P, P}=20.9 Hz), −11.72 (d, J_{P, P}=20.9 Hz); HRMS-ESI (m/z): [M+Na]+ calcd for C₁₆H₂₄N2NaO₁₆P₂ 585.05042; found 585.05105; spectral data are consistent with those reported by Bae et al.

Determination of Kinetic Parameters.

Assays were performed in a final volume of 500 μL of 50 mM Tris-HCl (pH 8.5) using 0.42 μM (10 μg) of enzyme (either wild-type or TDP-16). Reactions were prepared with either UDP (2.5 mM for wild-type, 1.0 mM for variant OleD), TDP (2.5 mM for wild-type, 2.0 mM for variant OleD), or 9 (20 mM) saturating and the corresponding reactant varied (from 0 mM until saturation conditions or solubility limits were met). Reactions were followed at 410 nm on a DU800 spectrophotometer (Beckman Coulter, Brea, Calif., USA) where the rate of 2-chloro-4-nitrophenolate formation was determined to be linear (<10 min). Initial rates were converted to product formation per unit time by comparing values to a standard curve. All experiments were performed in triplicate. Initial velocities were fit to the Michaelis-Menten equation using Origin Pro 7.0 software. OleD wild-type enzyme could not be saturated with donor 9 due to limited solubility (^{˜}25 mM under the stated conditions). Consequentially, kcat/Km for wild-type was determined by linear regression. Values obtained are in agreement with kinetic parameters from previous studies with OleD wildtype and numerous variants.

Determination of Equilibrium Constants.

Reactions contained 21 μM (200 μg) OleD variant P67T/S312F/A242L/Q268V, 1 mM UDP, and 1 mM β-D-glucopyranoside donor (1, 2, 4, 7, or 9) in Tris-HCl buffer (50 mM, pH 8.5) in a final total volume of 200 μl. Multiple time course evaluations were conducted to determine the time at which each reaction reached equilibrium (<2 min for 7, 9; <90 min for 1, 4; 200 min for 2). Following, each analysis was conducted in triplicate to the minimum observed time point for equilibrium and samples were processed and evaluated as described below to determine concentrations of UDP to 33a (UDP-α-D-glucose). HPLC conditions consisted of a Gemini-NX C-18 (5 μm, 250×4.6 mm) column (from Phenomenex, Torrance, Calif., USA) with a gradient of 0% B to 20% B over 20 min, 20% B to 80% B over 1 min, 80% B for 6 min (A=50 mM triethylammonium acetate buffer; B=acetonitrile; flow rate=1 mL min-1), and detection monitored at 254 nm. Glucoside and aglycon concentrations were inferred from the determined concentrations of UDP and 33a.

UDP-glucose pyrophosphorylase catalyzes the following reaction:

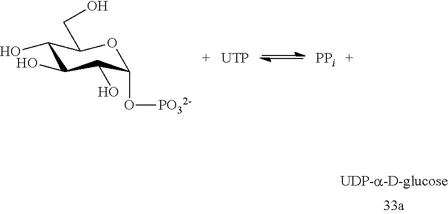

Glucosyltranserase GtfE catalyzes the following reaction:

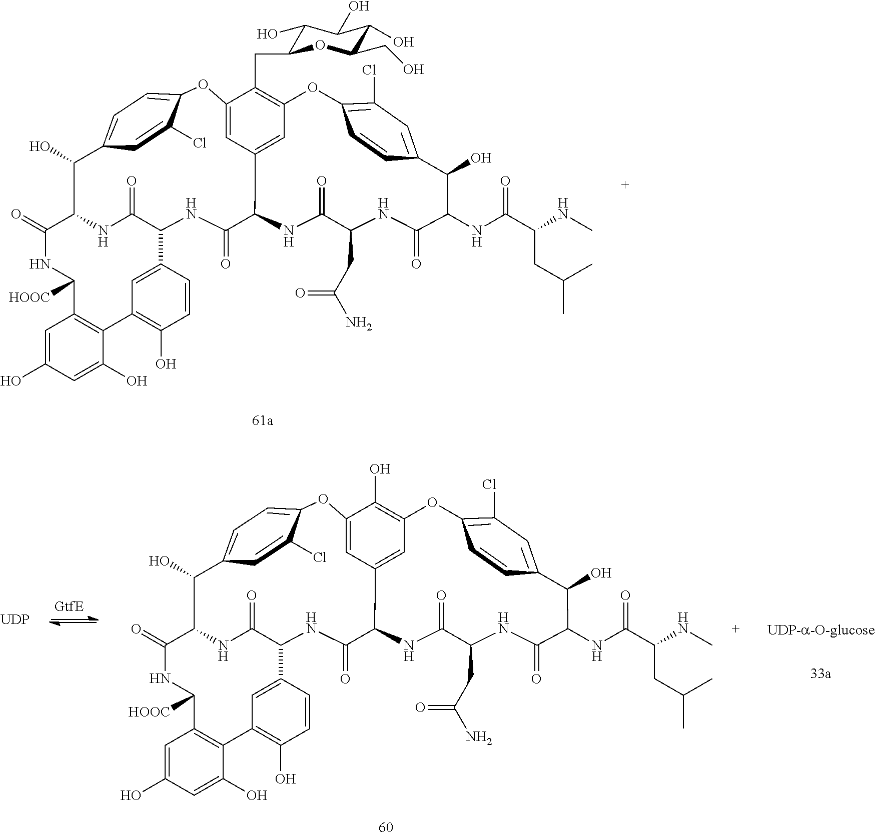

### Syntheses of 2-chloro-4-nitrophenyl glycosides

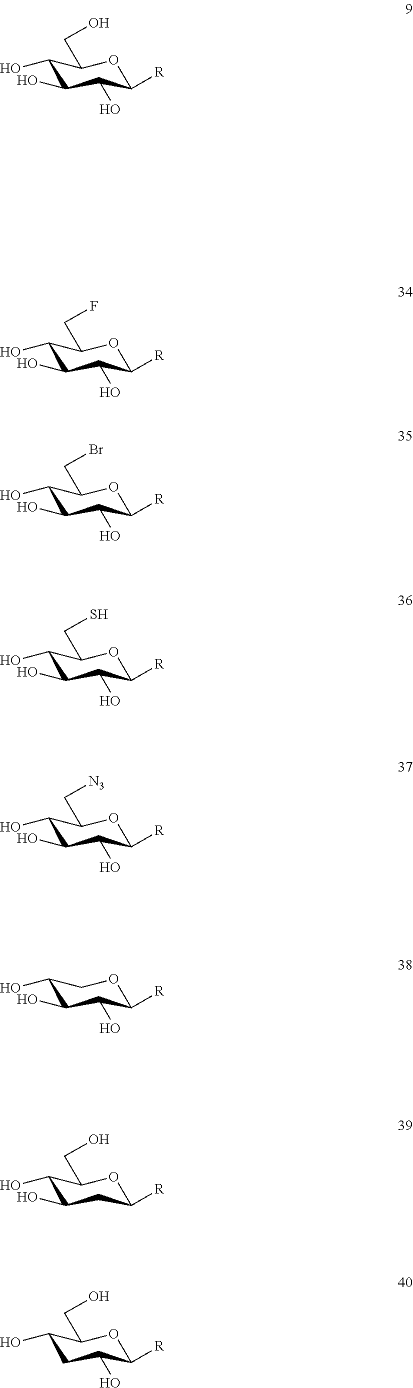

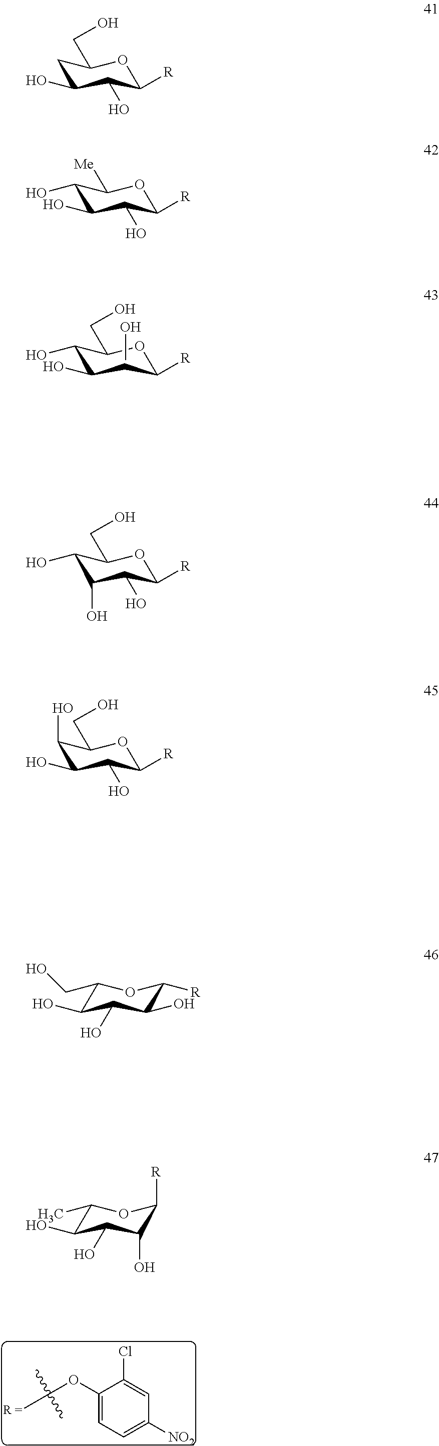

General procedure for bromination. Per O-acetylated glycopyranose (0.50 mmol) was dissolved in CH₂Cl₂ (1 mL) and treated with a 33% solution of HBr in glacial acetic acid (1 mL) at 0° C. for 30 min. The reaction was subsequently allowed to warm to room temperature and the stirring was continued until no starting compound was detected by TLC. Following, the mixture was diluted with CH2Cl2 (50 mL), washed with NaHCO3 sat solution (3×25 mL) and with brine (25 mL). The organic phase was dried over MgSO4, and the solvent removed under reduced pressure. The residue obtained was used directly without purification.

General Procedure for Phase Transfer Catalyzed Glycosylation of 2-chloro-4-nitrophenol.

Per Oacetylated glycopyranosyl bromide was dissolved in CH2Cl2 to a final concentration of 125 mM. Were successively added 1.5 equiv. of tetrabutylammonium bromide and 3 equiv. of 2-chloro-4-nitrophenol. An equal volume of 1M NaOH solution was added at 0° C. and the reaction mixture was stirred vigorously at room temperature overnight. After dilution with 2.5 volumes of EtOAc, the organic phase was washed three times with 0.2 volumes of a 1M NaOH solution and finally with 0.2 volumes of brine. The organic phase was dried over MgSO4, and the solvent removed under reduced pressure. Purification was carried out by chromatography on silica gel.

General Procedure for Deacetylation.

The acetylated glycoside (0.10 mmol) was dissolved in dry MeOH (2 mL) and treated at room temperature with a 0.1 M solution of sodium methoxide (150 μL). The mixture was stirred until no starting compound was detected by silica gel TLC with 9:1 (v/v) CH₂Cl₂/MeOH. Neutralization was then performed by adding Amberlite IR-120 (H+ form). The resin was removed via filtration and the solvent removed under reduced pressure. Purification was carried out by chromatography on silica gel.

### Synthesis of 2-chloro-4-nitrophenyl-β-D-glucopyranoside (9)

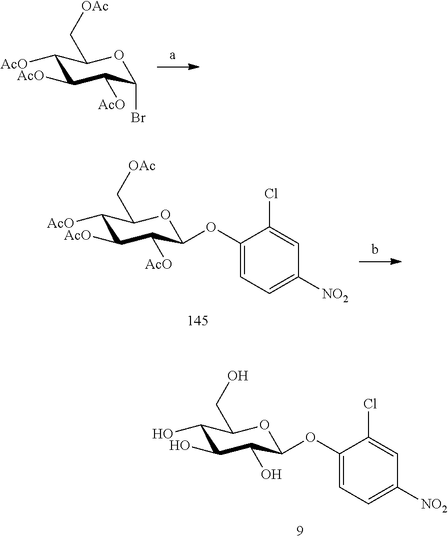

a) 2-chloro-4-nitrophenol, tetrabutylammonium bromide, CH2Cl2/NaOH (1:1), rt, 18 h; b) NaOMe 0.1M in MeOH, rt, 18 h.

(2-chloro-4-nitrophenyl)-2,3,4,6-tetra-O-acetyl-3-D-glucopyranoside (145).

This compound was prepared from 2,3,4,6 tetra-O-acetyl-α-D-glucopyranosyl bromide (1 g, 2.4 mmol) according the general procedure. The purification on silica gel (hexanes/EtOAc, 7:3) afforded 145 (1.08 mg, 89%) as a white powder. TLC Rf=0.45 (EtOAc/hexanes, 5:5); 1H NMR (400 MHz, CDCl₃) δ 8.30 (d, J_{H3′, H5′}=2.7 Hz, 1H, H-3′), 8.13 (dd, J_{H5′, H6′}=9.1 Hz, 1H, H-5′), 7.25 (d, 1H, H-6′), 5.38 (dd, J_{H1, H2}=7.5 Hz, J_{H2, H3}=9.4 Hz, 1H, H-2), 5.32 (dd, J_{H3, H4}=9.1 Hz, 1H, H-3), 5.19 (dd, J_{H4, H5}=9.9 Hz, 1H, H-4), 5.14 (d, 1 H, H-11), 4.27 (dd, J_{H5, H6a}=5.3 Hz, J_{H6a, H6b}=12.4 Hz, 1H, H-6a), 4.20 (dd, J_{H5, H6b}=2.6 Hz, 1H, H-6b), 3.92 (ddd, 1H, H-5), 2.10, 2.09, 2.07, 2.06 (4s, 12H, CH₃CO); 13C NMR (100 MHz, CDCl₃) δ 170.5, 170.3, 169.4, 169.2, (C═O), 157.4 (C-1′), 143.4 (C-4′), 126.4 (C-3′), 125.2 (C-2′), 123.7 (C-5′), 116.6 (C-6′), 99.4 (C-1), 72.7 (C-5), 72.2 (C-3), 70.6 (C-2), 68.1 (C-4), 61.9 (C-6), 20.8, 20.7, 20.7, 20.7 (CH₃CO); HRMS-ESI (m/z): [M+NH₄]+ calcd for C₂₀H₂₂ClNO₁₂, 521.1169; found 521.1158.

2-chloro-4-nitrophenyl-β-D-glucopyranoside (9).

A solution of 145 (750 mg, 1.49 mmol) was treated as described in the general procedure and purified by chromatography on silica gel (CH₂Cl₂/MeOH, 9:1) to give 9 (448 mg, 90%) as a white powder. TLC Rf=0.26 (CH₂Cl₂/MeOH, 9:1); 1H NMR (400 MHz, CD₃OD) δ 8.30 (d, J_{H3′, H5′}=2.8 Hz, 1H, H-3′), 8.18 (dd, J_{H5′, H6′}=9.2 Hz, 1H, H-5′), 7.41 (d, 1H, H-6′), 5.17 (d, J_{H1, H2}=7.6 Hz, 1H, H-1), 3.89 (dd, J_{H5, H6a}=2.2 Hz, J_{H6a, H6b}=12.1 Hz, 1H, H-6a), 3.70 (dd, J_{H5, H6b}=5.7 Hz, 1H, H-6b), 3.58 (dd, J_{H2, H3}=8.8 Hz, 1H, H-2), 3.55-3.50 (m, 1H, H-5), 3.50 (dd, J_{H3, H4}=8.8 Hz, 1H, H-3), 3.42 (dd, J_{H4, H5}=10.1 Hz, 1H, H-4); 13C NMR (100 MHz, CD₃OD) δ 159.4 (C-1′), 143.6 (C-4′), 126.7 (C-3′), 124.9 (C-2′), 124.8 (C-5′), 116.9 (C-6′), 101.8 (C-1), 78.6 (C-5), 78.1 (C-3), 74.6 (C-2), 71.0 (C-4), 62.4 (C-6). HRMS-ESI (m/z): [M+Na]+ calcd for C₁₂H₁₄ClNO₈, 358.0301; found 358.0313.

### Synthesis of (2-chloro-4-nitrophenyl)-6-deoxy-6-fluoro-3-D-glucopyranoside (34)

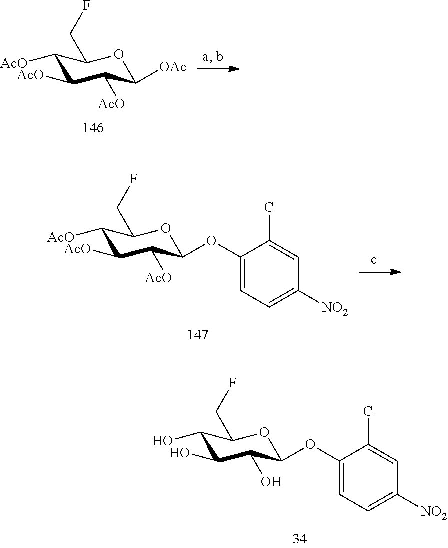

a) HBr in AcOH, CH₂Cl₂, 0° C., 1 h; b) 2-chloro-4-nitrophenol, tetrabutylammonium bromide, CH₂Cl₂/NaOH (1:1), rt, 18 h; c) NaOMe 0.1M in MeOH, rt, 12 h.

1,2,3,4,-tetra-O-acetyl-6-deoxy-6-fluoro-β-D-glucopyranose (146).

This compound was prepared as previously described from 1,2,3,4-tetra-O-acetyl-β-D-glucopyranose (300 mg, 0.86 mmol) in 83% yield (250 mg, 83%). TLC Rf=0.45 (EtOAc:hexanes, 5:5); 1H NMR (400 MHz, CDCl₃) δ 5.73 (d, J_{H1, H2}=8.2 Hz, 1H, H-1), 5.28 (dd, J_{H2, H3}=9.4 Hz, J_{H3, H4}=9.3 Hz, 1H, H-3), 5.18-5.09 (m, 2H, H-2, H-4), 4.48 (dddd, J_{H5, H6a}=2.4 Hz, J_{H5, H6b}=4.1 Hz, J_{H6a, H6b}=10.6 Hz, JH6, F=46.9 Hz, 2H, H-6a, H-6b), 3.83 (dddd, J_{H4, H5}=6.5 Hz, J_{H5, F}=22.4 Hz, 1H, H-5), 2.11, 2.05, 2.03, 2.02 (4s, 12H, CH₃CO); 13C NMR (100 MHz, CDCl₃) δ 170.2, 169.4, 169.3, 169.1 (C═O), 91.7 (C-1), 80.2 (d, J_{C6, F}=176.6 Hz, C-6), 73.5 (d, J_{C5, F}=19.5 Hz C-5), 72.9 (C-3), 70.3 (C-2), 67.7 (d, J_{C4, F}=6.5 Hz, C-4), 20.9, 20.7, 20.7 (CH₃CO).

(2-chloro-4-nitrophenyl)-2,3,4-tri-O-acetyl-6-deoxy-6-fluoro-β-D-glucopyranoside (147).

A solution of 146 (60 mg, 0.17 mmol) was treated as described in the general procedure. The residue obtained was directly used in the glycosylation reaction following the general procedure. The purification on silica gel (hexanes/EtOAc 7:3) afforded 147 (41 mg, 52%) as a white powder. TLC Rf=0.43 (EtOAc/hexanes, 5:5); 1H NMR (400 MHz, CDCl₃) δ 8.29 (d, J_{H3′, H5′}=2.7 Hz, 1H, H-3′), 8.13 (dd, J_{H5′, H6′}=9.1 Hz, 1H, H-5′), 7.28 (d, 1H, H-6′), 5.40-5.32 (m, 2H, H-2, H-3), 5.21 (d, J_{H1, H2}=7.2 Hz, 1H, H-1), 5.12 (dd, J_{H3, H4}=9.8 Hz, J_{H4, H5}=9.8 Hz, 1H, H-4), 4.58-4.45 (m, 2H, H-6a, H-6b), 3.99 (dddd, J_{H5, H6a}=1.8 Hz, J_{H5, H6b}=4.1 Hz, JH5, F=23.2 Hz, 1H, H-5), 2.08, 2.07, 2.05 (3s, 9H, CH₃CO); 13C NMR (100 MHz, CDCl₃) δ 170.4, 169.3, 169.0 (C═O), 157.2 (C-1′), 143.2 (C-4′), 126.1 (C-3′), 124.9 (C-2′), 123.7 (C-5′), 116.6 (C-6′), 99.2 (C-1), 80.5 (d, J_{C6, F}=176.1 Hz, C-6), 73.3 (d, J_{C5, F}=19.9 Hz C-5), 71.9 (C-3), 70.4 (C-2), 67.5 (d, JC4, F=7.0 Hz, C-4), 20.5, 20.5, 20.5 (CH₃CO); HRMS-ESI (m/z): [M+Na]+ calcd for C₁₈H₁₉ClFNO₁₀, 486.05737; found 486.05697.

(2-chloro-4-nitrophenyl)-6-deoxy-6-fluoro-3-D-glucopyranoside (34).

A solution of 147 (38 mg, 0.10 mmol) was treated as described in the general procedure and purified by chromatography on silica gel (CH₂Cl₂/MeOH, 9:1) to give 147 (26 mg, 93%) as a white powder. TLC Rf=0.40 (CH₂Cl₂/MeOH, 9:1); 1H NMR (400 MHz, CD₃OD) δ 8.31 (d, J_{H3′, H5′}=2.8 Hz, 1H, H-3′), 8.17 (dd, J_{H5′, H6′}=9.2 Hz, 1H, H-5′), 7.39 (d, 1H, H-6′), 5.20 (d, J_{H1, H2}=7.4 Hz, 1H, H-1), 4.64 (dddd, J_{H5, H6a}=1.8 Hz, J_{H5, H6b}=4.7 Hz, J_{H6a, H6b}=10.4 Hz, J_{H6, F}=47.7 Hz, 2H, H-6a, H-6b), 3.73 (dddd, J_{H4, H5}=9.7 Hz, J_{H5, F}=24.2 Hz, 1H, H-5), 3.57 (dd, J_{H2, H3}=8.9 Hz, 1H, H-2), 3.51 (dd, J_{H3, H4}=9.4 Hz, 1H, H-3), 3.45 (dd, 1H, H-4); 13C NMR (100 MHz, CD₃OD) δ 159.2 (C-1′), 143.7 (C-4′), 126.8 (C-3′), 124.9 (C-2′), 124.9 (C-5′), 116.7 (C-6′), 101.64 (C-1), 83.1 (d, JC6, F=172.0 Hz, C-6), 77.9 (C-3), 76.8 (d, J_{C5, F}=18.1 Hz C-5), 74.5 (C-2), 69.9 (d, J_{C4, F}=7.0 Hz, C-4); 19F NMR (376 MHz, CD₃OD) δ −236 (dt, J_{H5, F}=24.2 Hz, J_{H6, F}=47.7 Hz). HRMS-ESI (m/z): [M+H]+ calcd for C₁₂H₁₃ClFNO₇, 360.02568; found 360.02556.

### Synthesis of (2-chloro-4-nitrophenyl)-6-bromo-6-deoxy-3-D-glucopyranoside (35) and (2-chloro-4-nitrophenyl)-6-azido-6-deoxy-β-D-glucopyranoside (37)

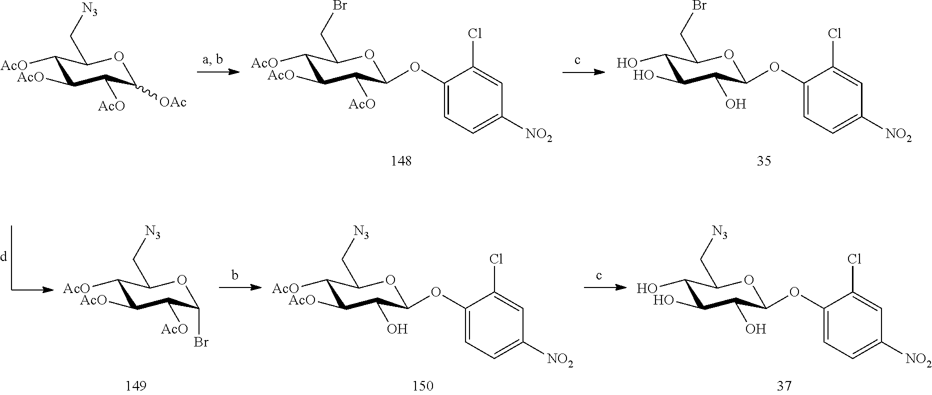

a)
HBr in AcOH, CH₂Cl₂, 0° C., 1 h; b) 2-chloro-4-nitrophenol, tetrabutylammonium bromide, CH₂Cl₂/NaOH (1:1), rt, 18 h; c) NaOMe 0.1M in MeOH, rt, 12 h; d) TiBr₄ in AcOEt/CH₂Cl₂, rt, 48 h;

(2-chloro-4-nitrophenyl)-2,3,4-tri-O-acetyl-6-bromo-6-deoxy-3-D-glucopyranose (148).

A solution of the previously described 1,2,3,4-tetra-O-acetyl-6-azido-6-deoxy-D-glucopyranose (200 mg, 0.54 mmol) in CH₂Cl₂ (500 μL) was treated as described in the general procedure. The residue obtained was directly used in the glycosylation reaction following the general procedure. The purification on silica gel (hexanes/EtOAc, 7:3) afforded 148 (135 mg, 48%) as a white powder. TLC Rf=0.60 (EtOAc:hexanes, 5:5). 1H NMR (400 MHz, CDCl₃) δ 8.28 (d, J_{H3′, H5′}=2.7 Hz, 1H, H-3′), 8.14 (dd, J_{H5′, H6′}=9.1 Hz, 1H, H-5′), 7.44 (d, 1H, H-6′), 5.38 (dd, J_{H1, H2}=7.6 Hz, J_{H2, H3}=9.5 Hz, 1H, H-2), 5.31 (dd, J_{H3, H4}=9.0 Hz, 1H, H-3), 5.13 (d, 1H, H-1), 5.04 (dd, J_{H4, H5}=9.8 Hz, 1H, H-4), 3.95-3.88 (m, 1H, H-5), 3.50 (dd, J_{H5, H6a}=2.5 Hz, J_{H6a, H6b}=11.4 Hz, 1H, H-6a), 3.41 (dd, J_{H5, H6b}=8.6 Hz, 1H, H-6b), 2.08, 2.07, 2.04 (3s, 9H, CH₃CO); 13C NMR (100 MHz, CDCl₃) δ 170.0, 169.4, 169.0 (C═O), 157.2 (C-1′), 143.3 (C-4′), 126.0 (C-3′), 124.8 (C-2′), 123.8 (C-5′), 116.9 (C-6′), 99.3 (C-1), 74.7 (C-5), 71.9 (C-3), 70.6 (C-2), 70.5 (C-4), 30.2 (C-6), 20.6, 20.6, 20.5 (CH₃CO); HRMS-ESI (m/z): [M+Na]+ calcd for C₁₈H₁₉BrClNO₁₀, 545.9779; found 545.9759.

(2-chloro-4-nitrophenyl)-6-bromo-6-deoxy-3-D-glucopyranose (35).

A solution of 148 (130 mg, 0.27 mmol) was treated as described in the general procedure 8.3 and purified by chromatography on silica gel (CH₂Cl₂/MeOH, 9:1) to give 35 (90 mg, 85%) as a white powder. TLC Rf=0.44 (CH₂Cl₂/MeOH, 9:1); 1H NMR (400 MHz, CD₃OD) δ 8.30 (d, J_{H3′, H5′}=2.8 Hz, 1H, H-3′), 8.17 (dd, J_{H5′, H6′}=9.2 Hz, 1H, H-5′), 7.47 (d, 1H, H-6′), 5.18 (d, J_{H1, H2}=7.6 HZ, 1H, H-1), 3.82 (dd, J_{H5, H6a}=2.1 Hz, J_{H6a, H6b}=11.0 Hz, 1H, H-6a), 3.71 (ddd, J_{H4, H5}=9.5 Hz, J_{H5, H6b}=7.4 Hz, 1H, H-5), 3.59 (dd, J_{H2, H3}=9.2 Hz, 1H, H-2), 3.54 (dd, 1H, H-6b), 3.50 (dd, J_{H3, H4}=9.1 Hz 1H, H-3), 3.37 (dd, 1H, H-4); 13C NMR (100 MHz, CD₃OD) δ 159.2 (C-1′), 143.7 (C-4′), 126.7 (C-3′), 124.8 (C-2′), 124.7 (C-5′), 117.2 (C-6′), 101.7 (C-1), 77.7 (C-3), 77.4 (C-5), 74.6 (C-2), 73.4 (C-4), 33.3 (C-6); HRMS-ESI (m/z): [M+H]+ calcd for C₁₂H₁₃BrClNO₇, 419.94561; found 419.94527.

1-bromo-2,3,4,-tri-O-acetyl-6-azido-6-deoxy-β-D-glucopyranose (149).

This compound was prepared from the previously described 1,2,3,4-tetra-O-acetyl-6-azido-6-deoxy-D-glucopyranose (200 mg, 0.54 mmol) which was dissolved in anhydrous CH₂Cl₂ (3.5 mL) and EtOAc (300 μL). Titanium tetrabromide (492 mg, 1.34 mmol) was added and the reaction was stirred at room temperature under nitrogen for 48 hours. The reaction was stopped by addition of NaOAc (200 mg) and stirred for 15 min, then filtered through Celite, and the Celite pad was washed with CH₂Cl₂ (20 mL). The organic filtrate was washed with cold water (15 mL) and the organic phase was then dried over MgSO₄, and the solvent removed under reduced pressure. Purification was carried out by chromatography on silica gel with hexanes/EtOAc 9:1 to 5:5 and afforded 149 (109 mg, 52%) as an oil. TLC Rf=0.68 (EtOAc/hexanes, 5:5); 1H NMR (400 MHz, CDCl₃) δ 6.63 (d, J_{H1, H2}=4.0 Hz, 1H, H-1), 5.55 (dd, J_{H2, H3}=9.8 Hz, J_{H3, H4}=9.6 Hz, 1H, H-3), 5.15 (dd, J_{H4, H5}=10.0 Hz, 1H, H-4), 4.84 (dd, 1H, H-2), 4.29-4.24 (m, 1H, H-5), 3.49 (dd, J_{H5, H6a}=2.7 Hz, J_{H6a, H6b}=13.7 Hz, 1H, H-6a), 3.36 (dd, J_{H5, H6b}=5.1 Hz, 1H, H-6b), 2.08, 2.06, 2.04 (3s, 9H, CH₃CO); 13C NMR (100 MHz, CDCl₃) δ 169.7, 169.6, 169.3 (C═O), 86.0 (C-1), 72.9 (C-5), 70.4 (C-3), 69.9 (C-2), 68.1 (C-4), 50.1 (C-6), 20.5, 20.5, 20.5 (CH₃CO); HRMS-ESI (m/z): [M+H]+ calcd for C₁₂H₁₆BrN₃O₇, 394.02444; found 394.02453.

(2-chloro-4-nitrophenyl)-2,3,4-tri-O-acetyl-6-azido-6-deoxy-β-D-glucopyranoside (150).

This compound was prepared following the general procedure from 149 (100 mg, 0.25 mmol). The purification on silica gel (hexanes/EtOAc, 7:3) afforded 150 (76 mg, 61%) as a white powder. TLC Rf=0.50 (EtOAc/hexanes, 5:5); 1H NMR (400 MHz, CDCl₃) δ 8.29 (d, J_{H3′, H5}=2.7 Hz, 1H, H-3′), 8.16 (dd, J_{H5′, H6′}=9.1 Hz, 1H, H-5′), 7.29 (d, 1H, H-6′), 5.40 (dd, J_{H1, H2}=7.6 Hz, J_{H2, H3}=9.5 Hz, 1H, H-2), 5.33 (dd, J_{H3, H4}=9.1 Hz, 1H, H-3), 5.19 (d, 1H, H-1), 5.10 (dd, J_{H4, H5}=9.9 Hz, 1H, H-4), 3.87 (ddd, J_{H5, H6a}=2.6 Hz, J_{H5, H6b}=7.5 Hz, 1H, H-5), 3.48 (dd, J_{H6a, H6b}=13.4 Hz, 1H, H-6a), 3.68 (dd, 1H, H-6b), 2.09, 2.08, 2.06 (3s, 9H, CH₃CO); 13C NMR (100 MHz, CDCl₃) δ 170.0, 169.4, 169.0 (C═O), 157.0 (C-1′), 143.3 (C-4′), 126.1 (C-3′), 124.9 (C-2′), 123.7 (C-5′), 116.7 (C-6′), 99.2 (C-1), 73.9 (C-5), 71.9 (C-3), 70.4 (C-2), 69.1 (C-4), 51.2 (C-6), 20.5, 20.5, 20.5 (CH₃CO); HRMS-ESI (m/z): [M+Na]+ calcd for C₁₈H₁₉ClN₄O₁₀, 509.06819; found 509.06898.

(2-chloro-4-nitrophenyl)-6-azido-6-deoxy-3-D-glucopyranoside (37).

A solution of 150 (40 mg, 0.08 mmol) was treated as described in the general procedure and purified by chromatography on silica gel (CH₂Cl₂/MeOH, 9:1) to give 37 (25 mg, 85%) as a white powder. TLC Rf=0.40 (CH₂Cl₂/MeOH, 9:1); 1H NMR (400 MHz, CD₃OD) δ 8.32 (d, J_{H3, H5′}=2.8 Hz, 1H, H-3′), 8.20 (dd, J_{H5′, H6′}=9.2 Hz, 1H, H-5′), 7.45 (d, 1H, H-6′), 5.24 (d, J_{H1, H2}=7.7 Hz, 1H, H-1), 3.71 (ddd, J_{H4, H5}=9.5 Hz, J_{H5, H6a}=2.4 Hz, J_{H5, H6b}=6.9 Hz, 1H, H-5), 3.60 (dd, J_{H2, H3}=9.1 Hz, 1H, H-2), 3.57 (dd, J_{H6a, H6b}=13.3 Hz, 1H, H-6a), 3.50 (dd, J_{H3, H4}=9.0 Hz, 1H, H3), 3.45 (dd, 1H, H-6b), 3.38 (dd, 1H, H-4); 13C NMR (100 MHz, CD₃OD) δ 159.1 (C-1′), 143.8 (C-4′), 126.8 (C-3′), 125.0 (C-2′), 124.9 (C-5′), 116.9 (C-6′), 101.6 (C-1), 77.7 (C-3), 77.4 (C-5), 74.6 (C-2), 72.0 (C-4), 52.7 (C-6); HRMS-ESI (m/z): [M+H]+ calcd for C₁₂H₁₃ClN₄O₇, 383.03650; found 383.03555.

### Synthesis of (2-chloro-4-nitrophenyl)-6-deoxy-6-thio-β-D-glucopyranoside (36)

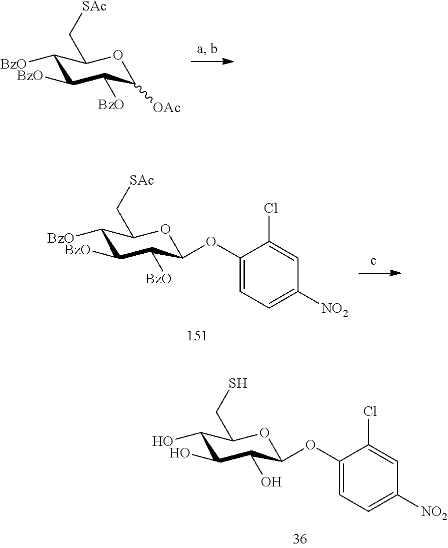

a) HBr in AcOH, CH₂Cl₂, 0° C., 1 h; b) 2-chloro-4-nitrophenol, tetrabutylammonium bromide, CH₂Cl₂/NaOH (1:1), rt, 18 h; c) NaOMe 0.1M in MeOH, rt, 12 h.

(2-chloro-4-nitrophenyl)-2,3,4-tri-O-benzoyl-6-S-acetyl-6-deoxy-β-D-glucopyranoside (151).

This compound was prepared following the general procedure from the previously described acetyl-2,3,4-tri-O-benzoyl-6-S-acetyl-6-deoxy-β-D-glucopyranoside (200 mg, 0.34 mmol). The purification on silica gel (hexanes/EtOAc, 7:3) afforded 151 (191 mg, 80%) as a white powder. TLC Rf=0.53 (EtOAc:hexanes, 5:5); 1H NMR (400 MHz, CDCl₃) δ 8.20 (d, J_{H3′, H5′}=2.7 Hz, 1H, H-3′), 8.01-7.85 (m, 6H, Ph), 7.55-7.26 (m, 10H, Ph), 5.95 (dd, J_{H2, H3}=J_{H3, H4}=9.5 Hz, 1H, H-3), 5.87 (dd, J_{H1, H2}=7.5 Hz, 1H, H-2), 5.60 (dd, J_{H4, H5}=9.5 Hz, 1H, H-4), 5.40 (d, 1H, H-1), 4.13-4.07 (m, 1H, H-5), 3.50 (dd, J_{H5, H6a}=2.9 Hz, J_{H6a, H6b}=14.4 Hz, 1H, H-6a), 3.09 (dd, J_{H5, H6b}=8.2 Hz, 1H, H-6b), 2.37 (CH₃CO); 13C NMR (100 MHz, CDCl₃) δ 194.1, 165.6, 165.4, 164.8 (C═O), 157.2 (C-1′), 143.2 (C-4′), 133.6, 133.4, 129.8, 129.8, 129.7, 129.7, 128.9, 128.6, 128.5, 128.5, 128.3, 128.3 (C-aro) 126.0 (C-3′), 125.1 (C-2′), 123.5 (C-5′), 116.9 (C-6′), 99.6 (C-1), 74.4 (C-5), 72.1 (C-3), 71.2 (C-2), 71.1 (C-4), 30.4 (C-6), 30.4 (CH₃CO); HRMS-ESI (m/z)[M+Na]+ calcd for C₃₅H₂₈ClNO₁₁, 728.0963; found 728.0944.

(2-chloro-4-nitrophenyl)-6-deoxy-6-thio-3-D-glucopyranoside (36).

A solution of 151 (100 mg, 0.14 mmol) was treated as described in the general procedure and purified by chromatography on silica gel (CH₂Cl₂/MeOH, 9:1) to give 36 (38 mg, 78%) as a white powder. TLC Rf=0.48 (CH₂Cl₂/MeOH, 9:1); 1H NMR (400 MHz, CD₃OD) δ 8.31 (d, J_{H3′, H5′}=2.7 Hz, 1H, H-3′), 8.18 (dd, J_{H5′, H6′}=9.2 Hz, 1H, H-5′), 7.46 (d, 1H, H-6′), 5.18 (d, J_{H1, H2}=7.7 Hz, 1H, H-1), 3.58 (dd, J_{H2, H3}=9.2 Hz, 1H, H-2), 3.57-3.52 (m, 1H, H-5), 3.49 (dd, J_{H3, H4}=9.1 Hz, 1H, H-3), 3.38 (dd, J_{H4, H5}=9.3 Hz, 1H, H-4), 2.99 (dd, 1H, J_{H5, H6a}=2.3 Hz, J_{H6a, H6b}=14.2 Hz, H-6a), 2.66 (dd, J_{H5, H6b}=7.9 Hz, 1H, H-6b); 13C NMR (100 MHz, CD₃OD) δ 159.2 (C-1′), 143.7 (C-4′), 126.8 (C-3′), 124.9 (C-2′), 124.9 (C-5′), 117.0 (C-6′), 101.8 (C-1), 78.8 (C-3), 77.8 (C-5), 74.7 (C-2), 73.5 (C-4), 26.8 (C-6) 4; HRMS-ESI (m/z): [M+H]+ calcd for C₁₂H₁₄ClNO₇S, 374.00717; found 374.00681.

### Synthesis of 2-chloro-4-nitrophenyl-β-D-xylopyranoside (38)

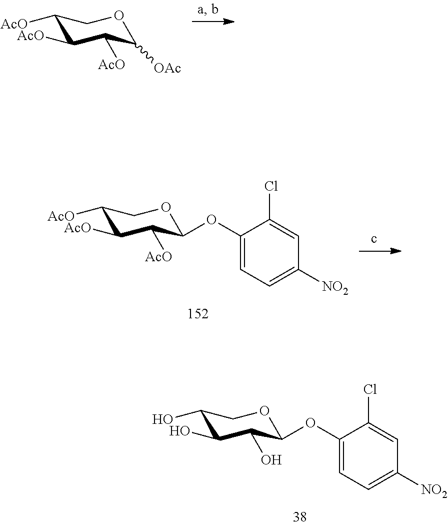

a) HBr in AcOH, CH₂Cl₂, 0° C., 2 h; b) 2-chloro-4-nitrophenol, tetrabutylammonium bromide, CH₂Cl₂/NaOH (1:1), rt, 18 h; c) NaOMe 0.1M in MeOH, rt, 12 h. 4 Less than 10% of dimer was observed by 1H NMR.

(2-chloro-4-nitrophenyl)-2,3,4-tri-O-acetyl-3-D-xylopyranoside (152).

A solution of per-O-acetylated Dxylopyranose (355 mg, 1.1 mmol) in CH₂Cl₂ was treated as described in the general procedure. The residue obtained was directly used in the glycosylation reaction following general procedure. Purification on silica gel (hexanes/EtOAc, 7:3) afforded 152 (180 mg, 38% yield) as a white powder. TLC Rf=0.56 (EtOAc:hexanes, 5:5). The product was carried forward without additional characterization.

2-chloro-4-nitrophenyl-3-D-xylopyranoside (38).

A solution of 152 (180 mg, 0.42 mmol) was treated as described in the general procedure 8.3 and purified by chromatography on silica gel (CH₂Cl₂/MeOH, 9:1) to give 38 (96 mg, 75% yield, 3-anomer) as white crystals. TLC Rf=0.53 (CH₂Cl₂/MeOH, 9:1); 1H NMR (400 MHz, CD₃OD) δ 8.31 (d, J_{H3′, H5′}=2.8 Hz, 1H, H-3′), 8.18 (dd, J_{H5′, H6′}=9.2 Hz, 1H, H-5′), 7.38 (d, 1H, H-6′), 5.15 (d, J_{H-1, H-2}=7.3 Hz, 1H, H-1), 3.95 (dd, J_{H-5a, H-4}=5.1 Hz, J_{H-5a, H-5b}=11.4 Hz, 1H, H-5a), 3.64-3.54 (m, 2H, H-2, H-4), 3.49-3.41 (m, 2H, H-3, H-5b); 13C NMR (100 MHz, CD₃OD) δ 159.3 (C-1′), 143.7 (C-4′), 126.8 (C-3′), 125.0 (C-2′), 124.9 (C-5′), 116.8 (C-6′), 102.5 (C-1), 77.6 (C-3), 74.4 (C-2), 70.8 (C-4), 67.2 (C-5). HRMS-ESI (m/z): [M+Na]+ calcd for C₁₂H₁₄ClNO₈, 358.0301; found 358.0313. An alternative reaction following sequential reaction procedures yielded 38 as a mixture of anomers. Subsequent HPLC purification of a portion of the crude product yielded 19 mg of α- and 68 mg of β-anomer, suggesting they were present in a α:β ratio of 1:3 in the crude reaction. Characterization of the α-anomer of 38 was as follows: TLC Rf=0.54 (CH₂Cl₂/MeOH, 9:1); 1H NMR (400 MHz, CD₃OD) δ 8.30 (d, J_{H3′, H5′}=2.8 Hz, 1H, H-3′), 8.19 (dd, J_{H5′, H6′}=9.2 Hz, 1H, H-5′), 7.45 (d, 1H, H-6′), 5.80 (d, J_{H-1, H-2}=3.5 Hz, 1H, H-1), 3.88 (dd, J_{H-5a, H-4}=8.5 Hz, J_{H-5a, H-5b}=9.8 Hz, 1H, H-5a), 3.66-3.56 (m, 3H, H-2, H-3, H-4), 3.48 (t, J_{H-5b, H-a}=9.8 Hz, 1H, H-5b).

### Synthesis of (2-chloro-4-nitrophenyl)-2-deoxy-3-D-glucopyranoside (39)

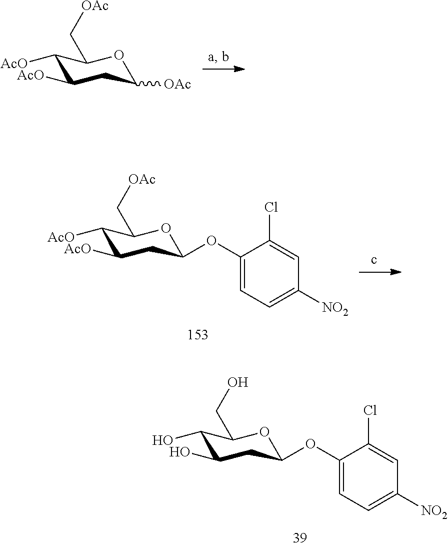

a) HBr in AcOH, CH2Cl2, 0° C., 1 h; b) 2-chloro-4-nitrophenol, tetrabutylammonium bromide, CH₂Cl₂/NaOH (1:1), rt, 18 h; c) NaOMe 0.1M in MeOH, rt, 12 h.

(2-chloro-4-nitrophenyl)-3,4,6-tri-O-acetyl-2-deoxy-3-D-glucopyranoside (153).

A solution of per-Oacetylated 2-deoxy-D-glucopyranose (150 mg, 0.45 mmol) in CH2Cl2 was treated as described in the general procedure. The residue obtained was then directly used in the glycosylation reaction following the general procedure. Purification on silica gel (hexanes/EtOAc, 8:2) afforded 153 (45 mg, 22%) as a white powder. TLC Rf=0.39 (EtOAc:hexanes, 5:5); 1H NMR (400 MHz, CDCl₃) δ 8.29 (d, J_{H3′, H5′}=2.7 Hz, 1H, H-3′), 8.12 (dd, J_{H5′, H6′}=9.1 Hz, 1H, H-5′), 7.25 (d, 1H, H-6′), 5.37 (dd, J_{H1, H2a}=2.5 Hz, J_{H1, H2b}=8.8 Hz, 1H, H-1), 5.17-5.11 (m, 1H, H-3), 5.09 (dd, J_{H3, H4}=J_{H4, H5}=8.8 Hz, 1H, H-4), 4.31 (dd, J_{H5, H6a}=5.8 Hz, J_{H6a, H6b}=12.2 Hz, 1H, H-6a), 4.19 (dd, J_{H5, H6b}=2.9 Hz, 1H, H-6b), 3.87 (ddd, 1H, H-5), 2.65 (ddd, J_{H1, H2a}=2.5 Hz, J_{H2a, H3}=4.7 Hz, J_{H2a, H2b}=12.9 Hz, 1H, H-2a), 2.26-2.18 (m, 1H, H-2b), 2.09, 2.09, 2.06 (3s, 9H, CH₃CO); 13C NMR (100 MHz, CDCl₃) δ 170.4, 170.1, 169.6, (C═O), 157.2 (C-1′), 142.6 (C-4′), 126.1 (C-3′), 124.4 (C-2′), 123.4 (C-5′), 115.6 (C-6′), 97.0 (C-1), 72.6 (C-5), 69.2 (C-3), 68.2 (C-4), 62.3 (C-6), 34.8 (C-2), 20.8, 20.7, 20.6, (CH₃CO); HRMS (m/z): [M+Na]+ calcd for C₁₈H₂ClNO₁₀, 468.0668; found 468.0689.

(2-chloro-4-nitrophenyl)-2-deoxy-β-D-glucopyranoside (39).

A solution of 153 (43 mg, 0.10 mmol) was treated as described in the general procedure and purified by chromatography on silica gel (CH₂Cl₂/MeOH, 95:5) to give 39 (19 mg, 61%) as a white powder. TLC Rf=0.45 (CH₂Cl₂/MeOH, 9:1); 1H NMR (400 MHz, CD₃OD) δ 8.28 (d, J_{H3′, H5′}=2.8 Hz, 1H, H-3′), 8.16 (dd, J_{H5′, H6′}=9.2 Hz, 1H, H-5′), 7.42 (d, 1H, H-6′), 5.46 (dd, J_{H1, H2a}=2.2 Hz, J_{H1, H2b}=9.7 Hz, 1H, H-1), 3.91 (dd, J_{H5, H6a}=2.3 Hz, J_{H6a, H6b}=12.0 Hz, 1H, H-6a), 3.71 (dd, J_{H5, H6b}=5.8 Hz, 1H, H-6b), 3.69 (ddd, J_{H2a, H3}=5.1 Hz, J_{H2b, H3}=J_{H3, H4}=12.0 Hz, 1 H, H-3), 3.45 (ddd, J_{H4, H5}=12.0 Hz, 1H, H-5), 3.31 (dd, 1H, H-4), 2.40 (ddd, J_{H1, H2a}=2.1 Hz, J_{H2a, H3}=5.1 Hz, J_{H2a, H2b}=12.3 Hz, 1H, H-2a), 1.86 (ddd, J_{H1, H2b}=9.7 Hz, J_{H2b, H3}=12.0 Hz, 1H, H-2b); 13C NMR (100 MHz, CD₃OD) δ 159.1 (C-1′), 143.5 (C-4′), 126.6 (C-3′), 124.9 (C-2′), 124.5 (C-5′), 117.1 (C-6′), 98.8 (C-1), 78.7 (C-5), 72.6 (C-4), 72.1 (C-3), 62.6 (C-6), 39.7 (C-2); HRMS-ESI (m/z): [M+H]+ calcd for C₁₂H₁₄ClNO₇, 342.03510; found 342.03495.

### Synthesis of (2-chloro-4-nitrophenyl)-3-deoxy-β-D-glucopyranoside (40)

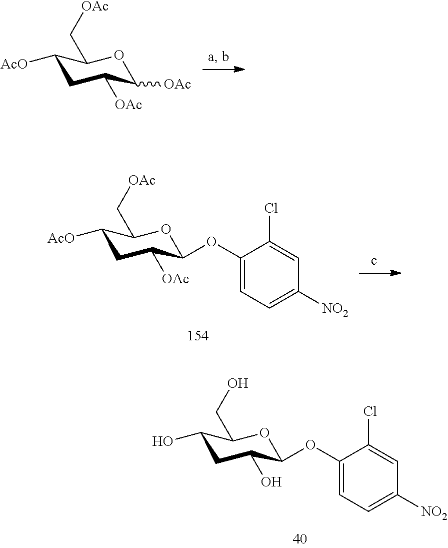

a) HBr in AcOH, CH2Cl2, 0° C., 1 h; b) 2-chloro-4-nitrophenol, tetrabutylammonium bromide, CH2Cl2/NaOH (1:1), rt, 18 h; c) NaOMe 0.1M in MeOH, rt, 12 h.

(2-chloro-4-nitrophenyl)-2,4,6-tri-O-acetyl-3-deoxy-β-D-glucopyranoside (154).

A solution of 1,2,4,6-tetra-O-acetyl-3-deoxy-β-D-glucopyranoside synthesized as previously described (60 mg, 0.18 mmol) in CH2Cl2 was treated as described in the general procedure. The residue obtained was used without further purification following the general procedure. The purification on silica gel (hexanes/EtOAc, 8:2) afforded 154 (41 mg, 50%) as a white powder. TLC Rf=0.49 (EtOAc:hexanes, 5:5); 1H NMR (400 MHz, CDCl₃) δ 8.28 (d, J_{H3′, H5′}=2.7 Hz, 1H, H-3′), 8.11 (dd, J_{H5′, H6′}=9.1 Hz, 1H, H-5′), 7.26 (d, 1H, H-6′), 5.21-5.17 (m, 2H, H-1, H-2), 4.94 (ddd, J_{H3a, H4}=4.9 Hz, J_{H3b, H4}=9.9 Hz, J_{H4, H5}=8.9 Hz, 1H, H-4), 4.25 (dd, J_{H5, H6a}=3.5 Hz, J_{H6a, H6b}=12.2 Hz, 1H, H-6a), 4.19 (dd, J_{H5, H6b}=5.8 Hz, 1H, H-6b), 3.94 (ddd, 1H, H-5), 2.67 (ddd, J_{H2, H3a}=12.7 Hz, J_{H3a, H3b}=9.5 Hz, J_{H3a, H3b}=9.5 Hz, 1H, H-3a), 2.08, 2.08, 2.05 (3s, 9H, CH₃CO), 1.80 (m, 1H, H-3b); 13C NMR (100 MHz, CDCl₃) δ 170.4, 169.4, 169.3, (C═O), 157.3 (C-1′), 142.8 (C-4′), 126.1 (C-3′), 124.7 (C-2′), 123.5 (C-5′), 115.9 (C-6′), 99.9 (C-1), 75.4 (C-5), 67.3 (C-2), 65.2 (C-4), 62.4 (C-6), 31.7 (C-3), 20.8, 20.8, 20.6, (CH₃CO); HRMS-ESI (m/z): [M+H]+ calcd for C₁₈H₂₀ClNO₁₀, 468.06680; found 468.06699.

(2-chloro-4-nitrophenyl)-3-deoxy-(3-D-glucopyranoside (40).

A solution of 154 (40 mg, 0.09 mmol) was treated as described in the general procedure and purified by chromatography on silica gel (CH₂Cl₂/MeOH, 95:5) to give 40 (25 mg, 86%) as a white powder. TLC Rf=0.50 (CH₂Cl₂/MeOH, 9:1); 1H NMR (400 MHz, CD₃OD) δ 8.29 (d, J_{H3′, H5′}=2.8 Hz, 1H, H-3′), 8.18 (dd, J_{H5′, H6′}=9.2 Hz, 1H, H-5′), 7.43 (d, 1H, H-6′), 5.12 (d, J_{H1, H2}=7.5 Hz, 1H, H-1), 3.87 (dd, J_{H5, H6a}=2.5 Hz, J_{H6a, H6b}=12.1 Hz, 1H, H-6a) 3.80 (ddd, J_{H2, H3a}=12.4 Hz, J_{H2, H3b}=11.8 Hz, 1H, H-2), 3.67 (dd, J_{H5, H6b}=5.9 Hz, 1H, H-6b), 3.68-3.61 (m, 1H, H-4), 3.50 (ddd, JH4, H5=8.9, H, H-5), 2.42 (ddd, J_{H3a, H4}=4.9 Hz, J_{H3a, H3b}=9.8 Hz, 1H, H-3a), 1.64 (ddd, J_{H3b, H4}=11.4 Hz, 1H, H-3b); 13C NMR (100 MHz, CD₃OD) δ 159.4 (C-1′), 143.5 (C-4′), 126.7 (C-3′), 124.9 (C-2′), 124.8 (C-5′), 116.9 (C-6′), 103.7 (C-1), 82.3 (C-5), 68.7 (C-2), 65.6 (C-4), 62.4 (C-6), 40.5 (C-3); HRMS-ESI (m/z): [M+H]+ calcd for C₁₂H₁₄ClNO₇, 342.03510; found 342.03511.

### Synthesis of (2-chloro-4-nitrophenyl)-4-deoxy-β-D-glucopyranoside (41)

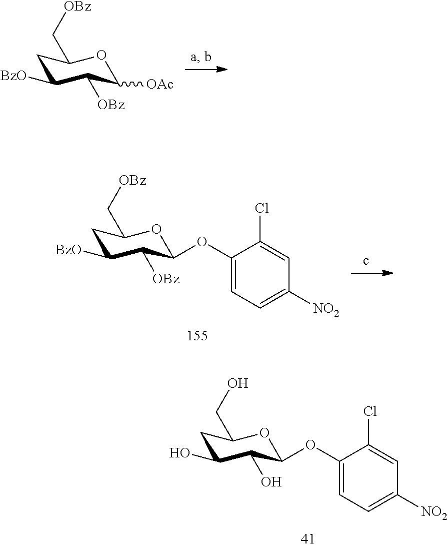

a) HBr in AcOH, CH2Cl2, 0° C., 1 h; b) 2-chloro-4-nitrophenol, tetrabutylammonium bromide, CH₂Cl₂/NaOH (1:1), rt, 18 h; NaOMe 0.1M in MeOH, rt, 16 h.

(2-chloro-4-nitrophenyl)-2,3,6-tri-O-benzoyl-4-deoxy-β-D-glucopyranoside (155).

A solution of acetyl 2,3,6-tri-O-benzoyl-4-deoxy-β-D-glucopyranoside (130 mg, 0.25 mmol) in CH₂Cl₂ was treated as described in the general procedure. The residue obtained was used without further purification following the general procedure. Purification on silica gel (hexanes/EtOAc, 9:1) afforded 155 (156 mg, 98%) as a white powder. TLC Rf=0.46 (EtOAc:hexanes, 1:2); 1H NMR (400 MHz, CDCl₃) δ 8.12 (d, J_{H3′, H5′}=2.7 Hz, 1H, H-3′), 8.05-7.97 (m, 6H, Ph), 7.80 (dd, J_{H5′, H6′}=9.1 Hz, 1H, H-5′), 7.64-7.34 (m, 9 H, Ph), 7.26 (d, 1H, H-6′), 5.82 (dd, J_{H1, H2}=7.5 Hz, J_{H2, H3}=9.3 Hz, 1H, H-2), 5.58 (ddd, J_{H3, H4a}=5.4 Hz, J_{H3, H4b}=11.1 Hz, 1H, H-3), 5.38 (d, 1H, H-11), 4.60 (dd, J_{H5, H6a}=3.7 Hz, J_{H6a, H6b}=11.8 Hz, 1H, H-6a), 4.53 (dd, J_{H5, H6b}=6.9 Hz, 1H, H-6b), 4.36-4.31 (m, 1H, H-5), 2.60 (ddd, J_{H4a, H4b}=11.5 Hz, J_{H4a, H5}=7.3 Hz, 1H, H-4a), 2.04 (m, 1H, H-4b); 13C NMR (100 MHz, CDCl₃) δ 165.8, 165.7, 165.1, (C═O), 157.3 (C-1′), 142.7 (C-4′), 133.5, 133.4, 133.3, 129.7, 129.6, 129.5, 129.3, 129.1, 129.0, 128.5, 128.4, 128.3 (C-aro), 125.9 (C-3′), 124.7 (C-2′), 123.2 (C-5′), 116.4 (C-6′), 99.6 (C-1), 71.6 (C-5), 70.7 (C-2), 70.6 (C-3), 65.2 (C-6), 32.0 (C-4). HRMS-ESI (m/z): [M+H]+ calcd for C₃₃H₂₆ClNO₁₀, 654.11374; found 654.11225.

(2-chloro-4-nitrophenyl)-4-deoxy-β-D-glucopyranoside (41).

A solution of 155 (150 mg, 0.09 mmol) was treated as described in the general procedure and purified by chromatography on silica gel (CH₂Cl₂/MeOH, 9:1) to give 41 (62 mg, 82%) as a white powder. TLC Rf=0.42 (CH₂Cl₂/MeOH, 9:1); 1H NMR (400 MHz, CD₃OD) δ 8.30 (d, J_{H3′, H5′}=2.8 Hz, 1H, H-3′), 8.17 (dd, J_{H5′, H6′}=9.2 Hz, 1H, H-5′), 7.42 (d, 1H, H-6′), 5.11 (d, J_{H1, H2}=7.6 Hz, 1H, H-1), 3.84-3.77 (m, 1H, H-5), 3.74 (ddd, J_{H2, H3}=9.0 Hz, J_{H3, H4a}=5.2 Hz, J_{H3, H4b}=11.5 Hz, 1H, H-3), 3.62 (dd, J_{H5, H6a}=4.0 Hz, J_{H6a, H6b}=11.8 Hz, 1H, H-6a), 3.58 (dd, J_{H5, H6b}=5.9 Hz, 1H, H-6b), 3.47 (dd, 1H, H-2), 2.00 (ddd, J_{H4a, H5}=1.9 Hz, J_{H4a, H4b}=12.9 Hz, 1H, H-4-a), 1.52 (dd, J_{H4b, H5}=11.7 Hz, 1H, H-4-b); 13C NMR (100 MHz, CD₃OD) δ 159.5 (C-1′), 143.5 (C-4′), 126.7 (C-3′), 124.9 (C-2′), 124.8 (C-5′), 116.9 (C-6′), 102.3 (C-1), 76.3 (C-2), 74.8 (C-5), 72.1 (C-3), 65.2 (C-6), 35.9 (C-4); HRMS-ESI (m/z): [M+H]+ calcd for C₁₂H₁₄ClNO₇, 342.03510; found 342.03525.

### Synthesis of (2-chloro-4-nitrophenyl)-6-deoxy-β-D-glucopyranoside (42)

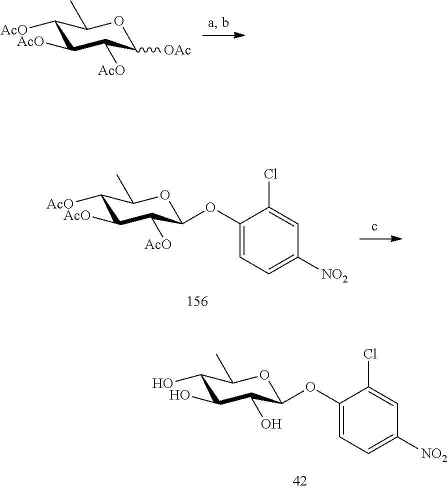

a) HBr in AcOH, CH2Cl2, 0° C., 1 h; b) 2-chloro-4-nitrophenol, tetrabutylammonium bromide, CH₂Cl₂/NaOH (1:1), rt, 18 h; c) NaOMe 0.1M in MeOH, rt, 12 h.

(2-chloro-4-nitrophenyl)-2,3,4-tri-O-acetyl-6-deoxy-3-D-glucopyranoside (156).

A solution of per-Oacetylated 6-deoxy-glucopyranose (120 mg, 0.37 mmol) in CH₂Cl₂ was treated as described in the general procedure. The residue obtained was directly used in the glycosylation reaction following the general procedure. Purification on silica gel (hexanes/EtOAc, 8:2) afforded 156 (132 mg, 81%) as a white powder. TLC Rf=0.55 (EtOAc/hexanes, 5:5); 1H NMR (400 MHz, CDCl₃) δ 8.25 (d, J_{H3′, H5′}=2.7 Hz, 1H, H-3′), 8.11 (dd, J_{H5′, H6′}=9.1 Hz, 1H, H-5′), 7.21 (d, 1H, H-6′), 5.34 (dd, J_{H1, H2}=7.7 Hz, J_{H2, H3}=9.7 Hz, 1H, H-2), 5.25 (dd, J_{H3, H4}=9.3 Hz, 1H, H-3), 5.12 (d, 1H, H-1), 4.93 (dd, J_{H4, H5}=9.5 Hz, 1H, H-4), 3.82-3.76 (m, 1H, H-5), 2.05, 2.05, 2.02 (3s, 9H, CH₃CO); 1.31 (d, J_{H5, H6}=6.2 Hz, 3H, H-6) 13C NMR (100 MHz, CDCl₃) δ 170.2, 169.5, 169.1, (C═O), 157.4 (C-1′), 143.0 (C-4′), 126.1 (C-3′), 124.8 (C-2′), 123.6 (C-5′), 116.2 (C-6′), 99.2 (C-1), 72.7 (C-3), 72.1 (C-4), 70.8 (C-2), 70.8 (C-5), 20.6, 20.6, 20.5, (CH₃CO), 17.4 (C-6); HRMS-ESI (m/z)[M+Na]+ calcd for C₁₈H₂₀ClNO₁₀, 468.0673; found 468.0677.

(2-chloro-4-nitrophenyl)-6-deoxy-β-D-glucopyranoside (42).

A solution of 156 (130 mg, 0.29 mmol) was treated as described in the general procedure 8.3 and purified by chromatography on silica gel (CH₂Cl₂/MeOH, 9:1) to give 42 (81 mg, 87%) as a white powder. TLC Rf=0.43 (CH₂Cl₂/MeOH, 9:1); 1H NMR (400 MHz, CD₃OD) δ 8.27 (d, J_{H3′, H5′}=2.8 Hz, 1H, H-3′), 8.16 (dd, J_{H5′, H6′}=9.2 Hz, 1H, H-5′), 7.35 (d, 1H, H-6′), 5.15 (dd, J_{H1, H2}=7.7 Hz, 1H, H-1), 3.62-3.55 (m, 2H, H-2, H-5), 3.46 (dd, J_{H2, H3}=J_{H3, H4}=9.1 Hz, 1H, H-3), 3.13 (dd, J_{H4, H5}=9.3 Hz, 1H, H-4), 1.31 (d, J_{H5, H6}=6.2 Hz, 3H, H-6); 13C NMR (100 MHz, CD₃OD) δ 159.3 (C-1′), 143.5 (C-4′), 126.8 (C-3′), 124.9 (C-2′), 124.8 (C-5′), 116.6 (C-6′), 101.5 (C-1), 77.7 (C-3), 76.4 (C-4), 74.8 (C-2), 73.8 (C-5), 18.0 (C-6); HRMS-ESI (m/z): [M+Na]+ calcd for C₁₂H₁₄ClNO₇, 342.0356; found 342.0356.

### Synthesis of 2-chloro-4-nitrophenyl-3-D-mannopyranoside (43)

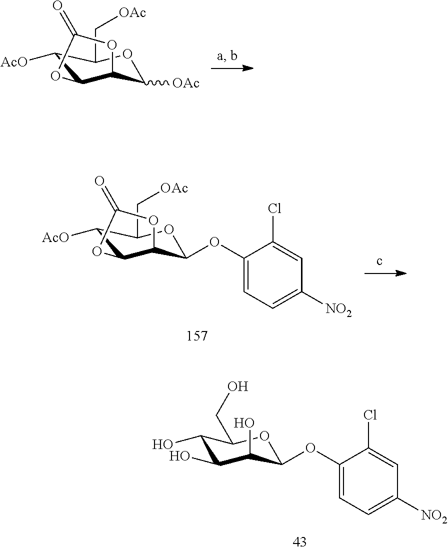

a) HBr in AcOH, CH₂Cl₂, 0° C., 90 min; b) potassium 2-chloro-4-nitrophenol, dicyclohexyl-18-crown-6, ACN, rt, 12 h; c) 4 Å molecular sieves, MeOH, rt, 12 h.

(2-chloro-4-nitrophenyl)-4,6-di-O-acetyl-2,3-carbonyl-β-D-mannopyranoside (157).

A solution of the previously described 4,6-di-O-acetyl-2,3-carbonyl-β-D-mannopyranoside (S18) (100 mg, 0.30 mmol) in CH₂Cl₂ was treated as described in the general procedure and the crude glycosyl bromide obtained used without further purification. For glycosylation, 2-chloro-4-nitrophenol (100 mg, 0.58 mmol) was converted to the potassium salt by dissolving it in an equimolar solution of potassium hydroxide, followed by evaporation under reduced pressure and finally freeze-drying. In a dry flask at room temperature were combined the above prepared crude glycosyl bromide, 2-chloro-4-nitrophenol (potassium salt, 94 mg, 0.45 mmol), dicyclohexyl-18-crown-6 (8 mg, 0.30 mmol) in anhydrous acetonitrile (3 mL). The reaction was stirred at room temperature for 12 hours. The mixture was filtered and the filtrate evaporated. The residue was diluted with CH2Cl2 (40 mL), washed with NaHCO3 sat solution (2×15 mL) and with brine (15 mL). The organic phase was dried over MgSO4, and the solvent removed under reduced pressure. Purification on silica gel (hexanes/EtOAc, 2:8) afforded 157 (117 mg, 87%) as a white powder. TLC Rf=0.38 (EtOAc:hexanes, 8:2); 1H NMR (400 MHz, CDCl₃) β 8.33 (d, J_{H3′, H5′}=2.7 Hz, 1H, H-3′), 8.17 (dd, J_{H5′, H6′}=9.1 Hz, 1H, H-5′), 7.28 (d, 1H, H-6′), 5.96 (dd, J_{H3, H4}=6.4 Hz, J_{H4, H5}=10.4 Hz, 1H, H-4), 5.85 (d, J_{H1, H2}=3.2 Hz, 1H, H-1), 5.08 (dd, J_{H2, H3}=9.2 Hz, 1H, H-2), 5.03 (dd, 1H, H-3), 4.17-4.03 (m, 3H, H-5, H-6a, H-6b), 2.13, 1.63 (2s, 6H, CH₃CO); 13C NMR (100 MHz, CDCl₃) δ 170.1, 168.5, (C═O), 156.1 (C-1′), 142.8 (C-4′), 126.0 (C-3′), 124.6 (C-2′), 123.6 (C-5′), 114.7 (C-6′), 93.0 (C-1), 76.7 (C-3), 71.6 (C-5), 70.9 (C-2), 66.0 (C-4), 61.4 (C-6), 20.6, 19.9 (CH₃CO); HRMS-ESI (m/z): [M+H]+ calcd for C₁₇H₁₆ClNO₁₁, 468.0304; found 468.0294.

2-chloro-4-nitrophenyl-(3-D-mannopyranoside (43).

A mixture of 157 (10 mg, 0.02 mmol) and powdered activated 4 Å molecular sieves (10 mg) in methanol (500 μL) was stirred at room temperature for 12 hours. The reaction was then filtered, concentrated and purified by chromatography on silica gel (CH₂Cl₂/MeOH, 9:1) to give 43 (5.5 mg, 73%) as a white powder. TLC Rf=0.39 (CH₂Cl₂/MeOH, 85:15); 1H NMR (500 MHz, CD₃OD) δ 8.30 (d, J_{H3′, H5′}=2.8 Hz, 1H, H-3′), 8.18 (dd, J_{H5′, H6′}=9.2 Hz, 1H, H-5′), 7.42 (d, 1H, H-6′), 5.40 (s, 1H, H-1), 4.15 (d, J_{H2, H3}=3.0 Hz, 1H, H-2), 3.92 (dd, J_{H5, H6a}=2.2 Hz, J_{H6a, H6b}=12.0 Hz, 1H, H-6a), 3.74 (dd, J_{H5, H6b}=6.1 Hz, 1H, H-6b), 3.67 (dd, J_{H3, H4}=J_{H4, H5}=9.5 Hz, 1H, H-4), 3.59 (dd, 1H, H-3), 3.47 (ddd, 1H, H-5); 13C NMR (125 MHz, CD₃OD) δ 159.2 (C-1′), 143.7 (C-4′), 126.7 (C-3′), 124.9 (C-2′), 124.8 (C-5′), 117.3 (C-6′), 99.7 (C-1), 79.0 (C-5), 75.0 (C-3), 72.1 (C-2), 68.2 (C-4), 62.7 (C-6). HRMS-ESI (m/z)[M+H]+ calcd for C₁₂H₁₄ClNO₈, 358.0300; found 358.0293.

### Synthesis of 2-chloro-4-nitrophenyl-3-D-allopyranoside (44)

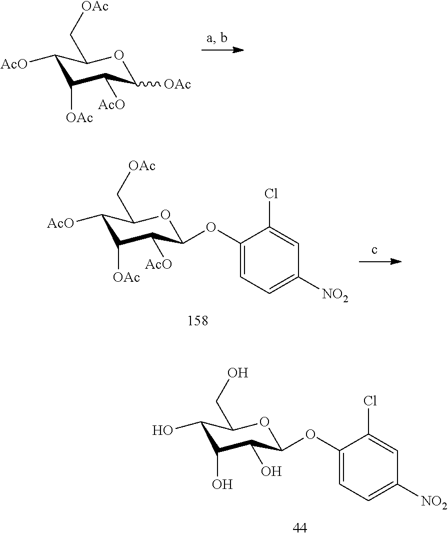

a) HBr in AcOH, CH2Cl2, 0° C., 90 min; b) 2-chloro-4-nitrophenol, tetrabutylammonium bromide, CH₂Cl₂/NaOH (1:1), rt, 18 h; c) NaOMe 0.1M in MeOH, rt, 12 h.

(2-chloro-4-nitrophenyl)-2,3,4,6-tetra-O-acetyl-β-D-allopyraniside (158).

A solution of per-O-acetylated allopyranose (172 mg, 0.44 mmol) in CH₂Cl₂ was treated as described in the general procedure. The residue obtained was directly used in the glycosylation reaction following the general procedure. Purification on silica gel (hexanes/EtOAc, 7:3) afforded 158 (63 mg, 29%) as a white powder. TLC Rf=0.37 (EtOAc/hexanes, 5:5); 1H NMR (400 MHz, CDCl₃) δ 8.30 (d, J_{H3′, H5′}=2.7 Hz, 1H, H-3′), 8.14 (dd, J_{H5′, H6′}=9.1 Hz, 1H, H-5′), 7.31 (d, 1H, H-6′), 5.75 (dd, J_{H2, H3}=3.0 Hz, J_{H3, H4}=2.9 Hz, 1H, H-3), 5.41 (d, J_{H1, H2}=8.1 Hz, 1H, H-1), 5.31 (dd, 1H, H-2), 5.06 (dd, J_{H4, H5}=9.6 Hz, 1H, H-4), 4.34-4.20 (m, 3H, H-5, H-6a, H-6b), 2.19, 2.11, 2.08, 2.04 (4s, 12H, CH₃CO); 13C NMR (100 MHz, CDCl₃) δ 170.5, 169.5, 168.9, 168.8, (C═O), 157.5 (C-1′), 142.9 (C-4′), 126.1 (C-3′), 124.8 (C-2′), 123.5 (C-5′), 116.2 (C-6′), 97.8 (C-1), 71.0 (C-5), 68.2 (C-3), 68.1 (C-2), 65.9 (C-4), 62.0 (C-6), 20.7, 20.6, 20.5, 20.4 (CH₃CO); HRMS-ESI (m/z): [M+Na]+ calcd for C₂₀H₂₂ClNO₁₂, 526.07227; found 526.07234.

2-chloro-4-nitrophenyl-3-D-allopyranoside (44).

A solution of 158 (50 mg, 0.10 mmol) was treated as described in the general procedure and purified by chromatography on silica gel (CH₂Cl₂/MeOH, 9:1) to give 44 (27 mg, 83%) as a white powder. TLC Rf=0.28 (CH₂Cl₂/MeOH, 9:1); 1H NMR (400 MHz, CD₃OD δ 8.29 (d, J_{H3′, H5′}=2.7 Hz, 1H, H-3′), 8.18 (dd, J_{H5′, H6′}=9.2 Hz, 1H, H-5′), 7.42 (d, 1 H, H-6′), 5.47 (dd, J_{H1, H2}=7.8 HZ, 1H, H-1), 4.16 (dd, J_{H2, H3}=3.0 Hz, J_{H3, H4}=2.9 Hz, 1H, H-3), 3.94 (ddd, J_{H4, H5}=9.7 Hz, J_{H5, H6a}=2.4 Hz, J_{H5, H6b}=5.7 Hz, 1H, H-5), 3.88 (dd, J_{H6a, H6b}=12.1 Hz, 1H, H-6a), 3.72 (dd, 1H, H-2), 3.70 (dd, 1H, H-6b), 3.63 (dd, 1H, H-4); 13C NMR (100 MHz, CD₃OD) δ 159.7 (C-1′), 143.5 (C-4′), 126.7 (C-3′), 124.9 (C-2′), 124.7 (C-5′), 116.8 (C-6′), 100.2 (C-1), 76.2 (C-5), 73.1 (C-3), 71.8 (C-2), 68.4 (C-4), 62.7 (C-6). HRMS-ESI (m/z): [M+Na]+ calcd for C₁₂H₁₄ClNO₈, 358.03002; found 358.03008.

### Synthesis of 2-chloro-4-nitrophenyl-(3-D-galactopyranoside (45)

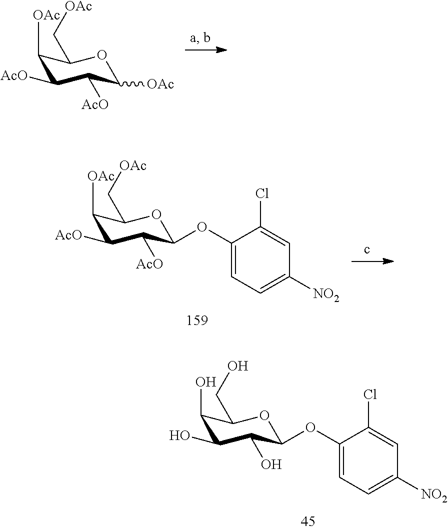

a) HBr in AcOH, CH2Cl2, 0° C., 1 h; b) 2-chloro-4-nitrophenol, tetrabutylammonium bromide, CH₂Cl₂/NaOH (1:1), rt, 18 h; c) Et3N/MeOH/H₂O (4:10:10), rt, 18 h.

(2-chloro-4-nitrophenyl)-2,3,4,6-tetra-O-acetyl-β-D-galactopyranoside (159).

A solution of per-acetylated galactopyranose (300 mg, 0.77 mmol) in was treated as described in the general procedure. The residue obtained was directly used in the glycosylation reaction following the general procedure. Purification on silica gel (hexanes/EtOAc, 7:3) afforded 159 (349 mg, 90%) as a white powder. TLC Rf=0.45 (EtOAc/hexanes, 5:5); 1H NMR (400 MHz, CDCl₃) δ 8.31 (d, J_{H3′, H5′}=2.5 Hz, 1H, H-3′), 8.13 (dd, J_{H5′, H6′}=9.2 Hz, 1H, H-5′), 7.29 (d, 1H, H-6′), 5.63 (dd, J_{H1, H2}=7.9 Hz, J_{H2, H3}=10.5 Hz, 1H, H-2), 5.50 (dd, J_{H3, H4}=3.4 Hz, J_{4, 5}=0.9 Hz, 1H, H-4), 5.15 (dd, 1H, H-3), 5.12 (d, 1H, H-1), 4.23 (dd, J_{H5, H6a}=7.0 Hz, J_{H6a, H6b}=11.0 Hz, 1H, H-6a), 4.17-4.13 (m, 1H, H-5), 4.14 (dd, J_{H5, H6b}=6.1 Hz, 1H, H-6b), 2.21, 2.10, 2.09, 2.03 (4s, 12H, CH₃CO); 13C NMR (100 MHz, CDCl₃) δ 170.2, 170.0, 169.9, 169.1, (C═O), 157.3 (C-1′), 143.1 (C-4′), 126.1 (C-3′), 124.8 (C-2′), 123.5 (C-5′), 116.3 (C-6′), 99.9 (C-1), 71.6 (C-5), 70.3 (C-3), 67.8 (C-2), 66.6 (C-4), 61.3 (C-6), 20.6, 20.6, 20.5, 20.5 (CH₃CO). HRMS-ESI (m/z): [M+Na]+ calcd for C₂₀H₂₂ClNO₁₂, 526.07227; found 526.07208. Spectral data are consistent with those previously reported.

2-chloro-4-nitrophenyl-β-D-galactopyranoside (45).

A solution of 159 (50 mg, 0.10 mmol) in 2.5 mL of a mixture Et3N/MeOH/H₂O (4:10:10) was stirred at room temperature for two hours. The reaction was concentrated under reduced pressure and purified by chromatography on silica gel (CH₂Cl₂/MeOH, 9:1) to give 45 (29 mg, 88%) as a white powder. TLC Rf=0.28 (CH₂Cl₂/MeOH, 9:1); 1H NMR (400 MHz, CD₃OD) δ 8.30 (d, J_{H3′, H5′}=2.8 Hz, 1H, H-3′), 8.17 (dd, J_{H5′, H6′}=9.2 Hz, 1H, H-5′), 7.45 (d, 1H, H-6′), 5.13 (dd, J_{H1, H2}=7.7 Hz, 1H, H-1), 3.95-3.87 (m, 2H, H-2, H-4), 3.80-3.73 (m, 3H, H-4, H-6a, H-6b), 3.62 (dd, J_{H2, H3}=9.7 Hz, J_{H3, H4}=3.4 Hz, 1H, H-3), 13C NMR (100 MHz, CD₃OD) δ 159.5 (C-1′), 143.6 (C-4′), 126.7 (C-3′), 124.9 (C-2′), 124.8 (C-5′), 116.9 (C-6′), 102.5 (C-1), 77.5 (C-5), 74.9 (C-3), 71.9 (C-2), 70.2 (C-4), 62.4 (C-6) HRMS-ESI (m/z): [M+Na]+ calcd for C₁₂H₁₄ClNO₈, 358.03002; found 358.02994. Spectral data are consistent with those previously reported.

### Synthesis of 2-chloro-4-nitrophenyl-β-L-glucopyranoside (46)

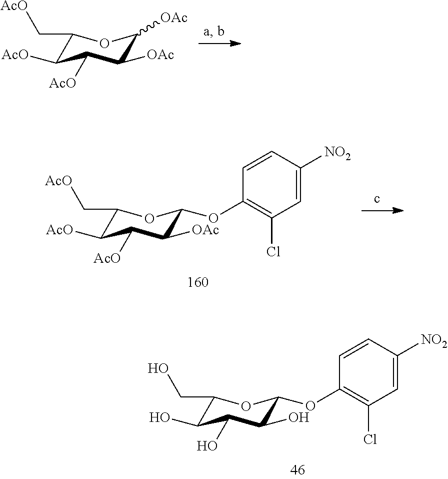

a) HBr in AcOH, CH2Cl2, 0° C., 90 min; b) 2-chloro-4-nitrophenol, tetrabutylammonium bromide, CH2Cl2/NaOH (1:1), rt, 18 h; c) NaOMe 0.1M in MeOH, rt, 18 h.

(2-chloro-4-nitrophenyl)-2,3,4,6-tetra-O-acetyl-3-L-glucopyranoside (160).

A solution of per-O-acetylated L-glucopyranose (150 mg, 0.39 mmol) in CH₂Cl₂ was treated as described in the general procedure. The residue obtained was directly used in the glycosylation reaction following the general procedure. Purification on silica gel (hexanes/EtOAc, 7:3) afforded 160 (95 mg, 49%) as a white powder. TLC Rf=0.46 (EtOAc/hexanes, 5:5); 1H NMR (400 MHz, CDCl₃) δ 8.30 (d, J_{H3′, H5′}=2.7 Hz, 1H, H-3′), 8.13 (dd, J_{H5′, H6′}=9.1 Hz, 1H, H-5′), 7.27 (d, 1H, H-6′), 5.40 (dd, J_{H1, H2}=7.5 Hz, J_{H2, H3}=9.3 Hz, 1H, H-2), 5.33 (dd, J_{H3, H4}=9.1 Hz, 1H, H-3), 5.19 (dd, J_{H4, H5}=9.8 Hz, 1H, H-4), 5.17 (d, 1H, H-1), 4.29 (dd, J_{H5, H6a}=5.2 Hz, J_{H6a, H6b}=12.4 Hz, 1H, H-6a), 4.22 (dd, J_{H5, H6b}=2.6 Hz, 1H, H-6b), 3.95 (ddd, 1H, H-5), 2.10, 2.09, 2.07, 2.06 (4s, 12H, CH₃CO); 13C NMR (100 MHz, CDCl₃) δ 170.3, 170.1, 169.2, 168.9, (C═O), 157.2 (C-1′), 143.2 (C-4′), 126.1 (C-3′), 124.9 C-2′), 123.5 (C-5′), 99.2 (C-1), 72.5 (C-5), 72; 1 (C-3), 70.4 (C-2), 67.9 (C-4), 61.7 (C-6), 20.6, 20.5, 20.5, 20.5 (CH₃CO); C₂₀H₂₂ClNO₁₂, 526.07227; found 526.07263.

2-chloro-4-nitrophenyl-3-L-glucopyranoside (46).

A solution of 160 (75 mg, 0.15 mmol) was treated as described in the general procedure and purified by chromatography on silica gel (CH₂Cl₂/MeOH, 9:1) to give 46 (35 mg, 70%) as a white powder. TLC Rf=0.26 (CH₂Cl₂/MeOH, 9:1); 1H NMR (400 MHz, CD₃OD) δ 8.30 (d, J_{H3′, H5′}=2.8 Hz, 1H, H-3′), 8.18 (dd, J_{H5′, H6′}=9.2 Hz, 1H, H-5′), 7.42 (d, 1H, H-6′), 5.17 (d, J_{H1, H2}=7.6 Hz, 1H, H-11), 3.89 (dd, J_{H5, H6a}=2.2 Hz, J_{H6a, H6b}=12.1 Hz, 1H, H-6a), 3.70 (dd, J_{H5, H6b}=5.7 Hz, 1H, H-6b), 3.58 (dd, J_{H2, H3}=9.0 Hz, 1H, H-2), 3.55-3.50 (m, 1H, H-5), 3.50 (dd, J_{H3, H4}=8.7 Hz, 1H, H-3), 3.42 (dd, J_{H4, H5}=9.4 Hz, 1H, H-4); 13C NMR (100 MHz, CD₃OD) δ 159.4 (C-1′), 143.6 (C-4′), 126.7 (C-3′), 124.9 (C-2′), 124.8 (C-5′), 116.9 (C-6′), 101.8 (C-1), 78.6 (C-5), 78.1 (C-3), 74.6 (C-2), 71.1 (C-4), 62.4 (C-6). HRMS-ESI (m/z): [M+H]+ calcd for C₁₂H₁₄ClNO₈, 358.03002; found 358.02997.

### Synthesis of 2-chloro-4-nitrophenyl-β-L-rhamnopyranoside (47)

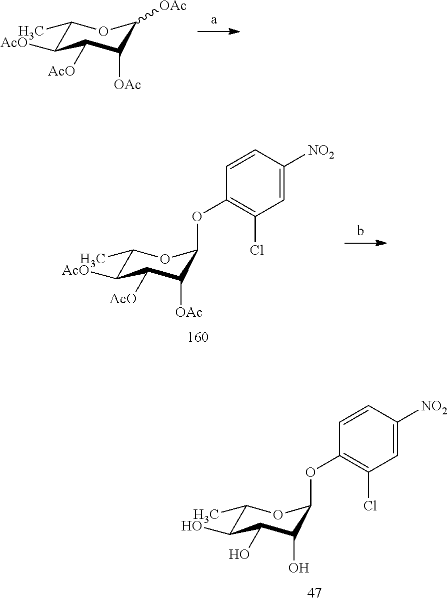

a) 2-chloro-4-nitrophenol, BF3.OEt2, Et3N, CH₂Cl₂, rt, 5 days; b) NaOMe 0.1M in MeOH, rt, 18 h.

2-chloro-4-nitrophenyl-2,3,4-tri-O-acetyl-β-L-rhamnopyranose (161).

To a solution of 1,2,3,4-tetra-Oacetyl-L-rhamnopyranose (530 mg, 1.50 mmol) in anhydrous CH2Cl2 (5 mL), were added 2 equiv. of 2-chloro-4-nitrophenol (520 mg), 1 equiv. of triethylamine, and 10 equiv. of boron trifluoride etherate in 4 mL of anhydrous CH₂Cl₂. The reaction was allowed to proceed for 5 days. Purification on silica gel (hexanes/EtOAc, 7:3) afforded 161 (557 mg, 83%) as a yellow powder. TLC Rf=0.68 (EtOAc:hexanes, 5:5). 1H NMR (400 MHz, CDCl₃) δ 8.33 (d, J_{H3′, H5′}=2.7 Hz, 1H, H-3′), 8.15 (dd, J_{H5′, H6′}=9.1 Hz, 1H, H-5′), 7.44 (d, 1H, H-6′), 5.65 (d, J_{H1, H2}=1.6 Hz, 1H, H-1) 5.57-5.51 (m, 2H, H-2, H-3), 5.20 (dd, J=9.7 Hz, 1H, H-4), 3.95-3.90 (m, 1H, H-5), 2.22, 2.08, 2.05 (3s, 9H, CH₃CO), 1.22 (d, J_{H5, H6}=6.2 Hz, 1H, H-6); 13C NMR (100 MHz, CDCl₃) δ 170.0, 169.9, 169.8 (C═O), 156.1 (C-1′), 142.7 (C-4′), 126.3 (C-3′), 124.6 (C-2′), 123.7 (C-5′), 115.0 (C-6′), 96.2 (C-1), 70.3 (C-4), 69.2 (C-2), 68.5 (C-3), 68.3 (C-5), 20.8, 20.8, 20.7 (CH₃CO), 17.4 (C-6); HRMS-ESI (m/z): [M+Na]+ calcd for C₁₈H₁₉ClNO₁₀, 468.06795; found 468.06597.

2-chloro-4-nitrophenyl-β-L-rhamnopyranose (47).

A solution of 161 (525 mg, 1.18 mmol) was treated as described in the general procedure and purified by chromatography on silica gel (CH₂Cl₂/MeOH, 9:1) to give 47 (327 mg, 87%) as a white powder. TLC Rf=0.39 (CH₂Cl₂/MeOH, 9:1); 1H NMR (400 MHz, CD₃OD) δ 8.28 (d, J_{H3′, H5′}=2.8 Hz, 1H, H-3′), 8.18 (dd, J_{H5′, H6′}=9.2 Hz, 1H, H-5′), 7.47 (d, 1H, H-6′), 5.71 (d, J_{H-1, H-2}=1.8, 1H, H-1), 4.13 (dd, J_{H-2, H-3}=3.4 Hz, 1H, H-2), 5.44 (dd, J_{H-3, H-4}=9.1 Hz, 1H, H-3), 3.58-3.49 (m, 2H, H-4, H-5), 1.24 (d, J_{H-5, H-5}=5.8 Hz, 3H, H-6); 13C NMR (100 MHz, CD₃OD) δ 156.9 (C-1′), 142.2 (C-4′), 125.6 (C-3′), 123.7 (C-2′), 123.7 (C-5′), 115.4 (C-6′), 99.3 (C-1), 72.2 (C-4), 70.9 (C-3), 70.5 (C-5), 70.4 (C-2), 16.8 (C-6); HRMS-ESI (m/z): [M+H]+ calcd for C₁₂H₁₄ClNO₇, 342.03510; found 342.03391.

2-chloro-4-nitrophenyl glycoside Screening and Data.

2-chloro-4-nitrophenyl glycoside Screening.

Reactions containing 7.0 μM (100 μg) of OleD variant TDP-16, 1 mM or 0.1 mM of (U/T)DP, and 1 mM of glycoside member (9, 34-47) in 50 mM Tris (pH 8.5) with a final volume of 300 μl were incubated at room temperature. Aliquots were removed at various time points, mixed with an equal volume of ddH₂O, frozen in a bath of dry ice and acetone, and stored at −20° C. Following, samples were thawed at 4° C. and filtered through a MultiScreen Filter Plate (from Millipore, Billerica, Mass., USA) according to manufacturer's instructions. Samples were evaluated for formation of NDPsugar by analytical reverse-phase HPLC with a 250 mm×4.6 mm Gemini-NX 5μ C18 column (from Phenomenex, Torrance, Calif., USA) using a linear gradient of 0% to 50% CH₃CN (solvent B) over 25 minutes (solvent A=50 mM PO₄⁻², 5 mM tetrabutylammonium bisulfate, 2% acetonitrile [pH adjusted to 6.0 with KOH]; flow rate=1 ml min⁻¹; A₂₅₄ nm). In the case of 51a and 50b formation, percent conversion was calculated from peak height due to co-elution of NDP and product. Screening of the α-anomer of 38 yielded no turnover in any reactions, demonstrating that TDP-16 is only capable of recognizing the β-anomers of D-sugars.

Purification and Characterization of NDP-Sugars.

Reactions containing 2.5 mM UDP or TDP, 1 mM 2-chloro-4-nitrophenyl glycoside (9, 34-42, or 44), 4.2 μM (20 μg) of OleD variant TDP-16, and 50 mM Tris (pH8.5, total volume of 100 μL) were prepared and allowed to proceed at room temperature for 12 hours. A volume of 100 μL of ddH₂O was added to each reaction and samples were filtered through a MultiScreen Filter Plate (from Millipore, Billerica, Md., USA) for 2 hours at 2000 g. The recovered filtrate for each sample was injected onto a Gemini-NX C-18 (5 μm, 250×4.6 mm) column (from Phenomenex, Torrance, Calif., USA) with a gradient of 0% to 20% CH₃CN (solvent B) over 20 min (A=50 mM triethylammonium acetate buffer; flow rate=1 mL min⁻¹; A₂₅₄ nm). Fractions corresponding to the desired products were collected, frozen, and lyophilized. Following, samples were dissolved in 1 mL of ddH₂O, frozen, and lyophilized (×3). Final products were dissolved in 1:1 ddH₂O/acetonitrile to a final concentration of 1 μg mL-1 and submitted for mass analysis.

Evaluation of Single Enzyme Coupled System.

Reactions containing 10.5 μM (50 kg) of purified OleD variant TDP-16, 1 mM of UDP, 1 mM 4-methylumbelliferone (58) and 1 mM of 2-chloro-4-nitrophenyl glycoside (9, 34-42, or 44) in Tris-HCl buffer (50 mM, pH 8.5) at a final volume of 100 μl were incubated in a 30° C. water bath for 24 hours. Samples were subsequently mixed with an equal volume of MeOH, centrifuged at 10,000 g for 30 min at 0° C., and the supernatant removed for analysis. The clarified reaction mixtures were analyzed by analytical reverse-phase HPLC. Fractions corresponding to the desired products were collected, frozen, lyophilized, dissolved in 1:1 acetonitrile/water to a final concentration of 1 μg mL⁻¹, and submitted for mass analysis.

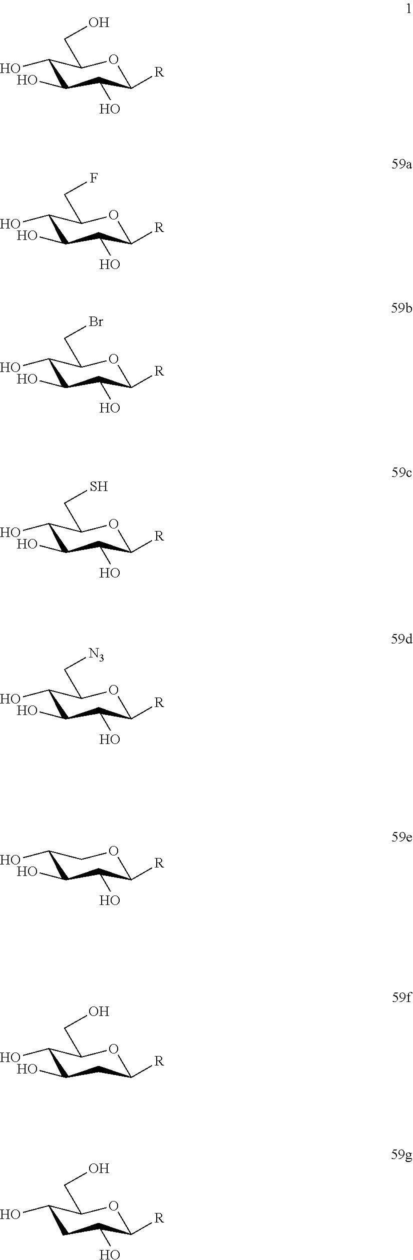

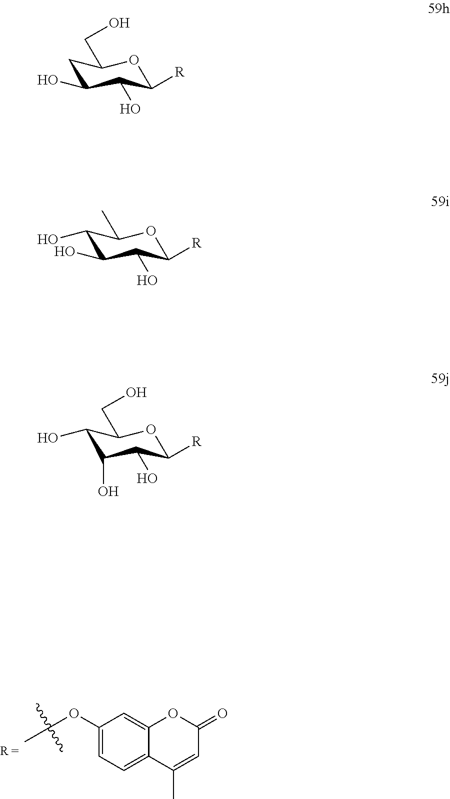

### Synthesis of vancomycin aglycon (60)

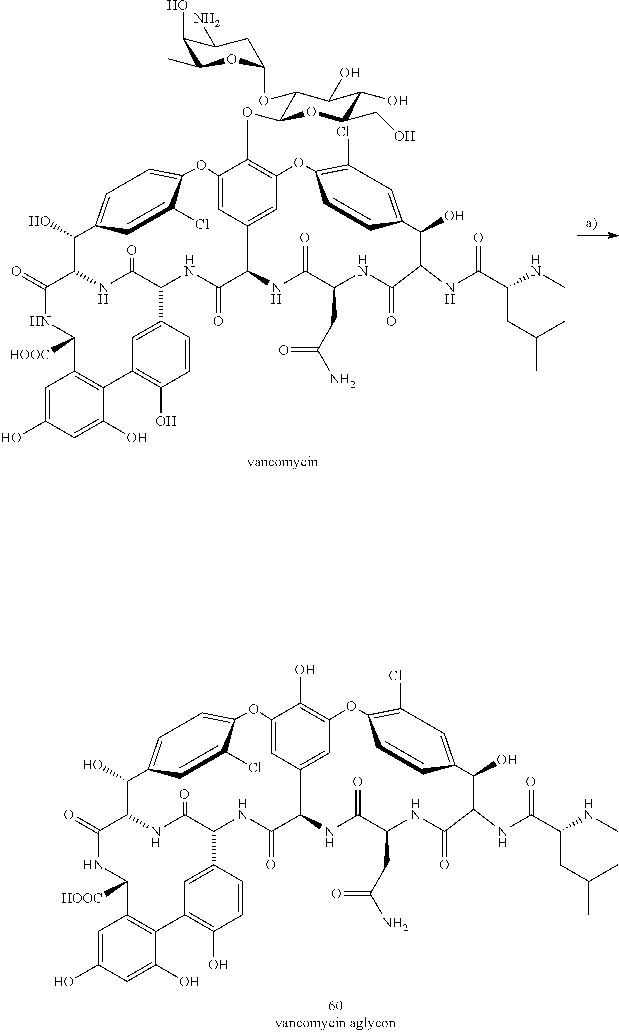

### A) HCL in H₂O, 10 min.

From vancomycin, 60 was prepared as described by Thompson, et al. Purification performed by analytical reverse-phase HPLC yielded 60 (>98% pure by peak area). HRMS-ESI (m/z): [M+H]+ calcd for C₅₃H₅₂C₁₂N₈O₁₇ 1143.2900; found 1143.2889.

General Reaction Procedure for Double Enzyme Coupled System.

All reactions were performed in a final volume of 100 μl Tris-HCl buffer (50 mM, pH 8.5) with 10.8 μM (50 μg) purified GtfE (see section 2), 1 mM vancomycin aglycon (60) and 1 mM of 2-chloro-4-nitrophenyl glycoside (9, 34-42, or 44). Reactions with 35-38, 40-41, or 44 as donor contained 10.5 μM (50 μg) OleD variant TDP-16 and 1 mM UDP. Reactions with 9, 34, or 39 as donor contained 1.1 μM (5 μg) OleD variant TDP-16 and 1 μM UDP. A reaction with 42 as donor contained 0.1 μM (0.5 μg) OleD variant TDP-16 and 0.001 mM UDP. All components of the reaction(s) were added at time equals zero hours. Reactions were then incubated in a 30° C. water bath for 24 hours. Samples were then prepared and analyzed as described above.

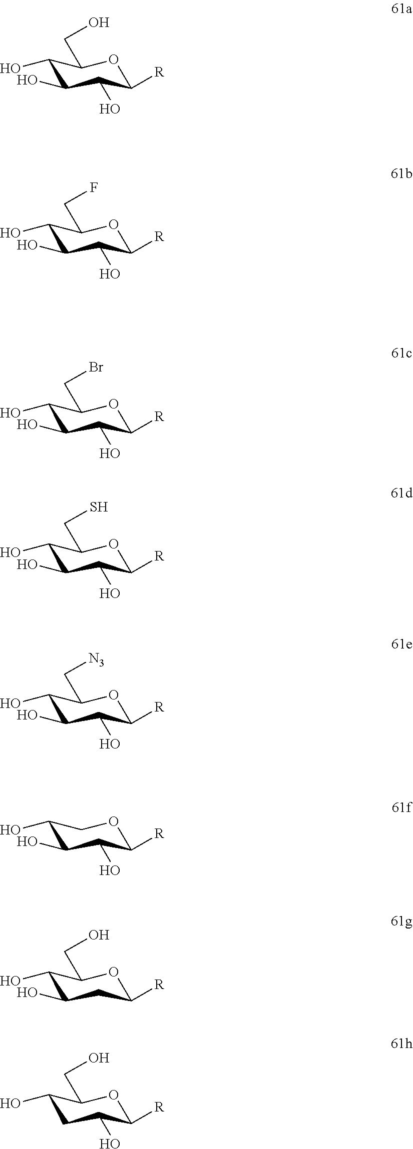

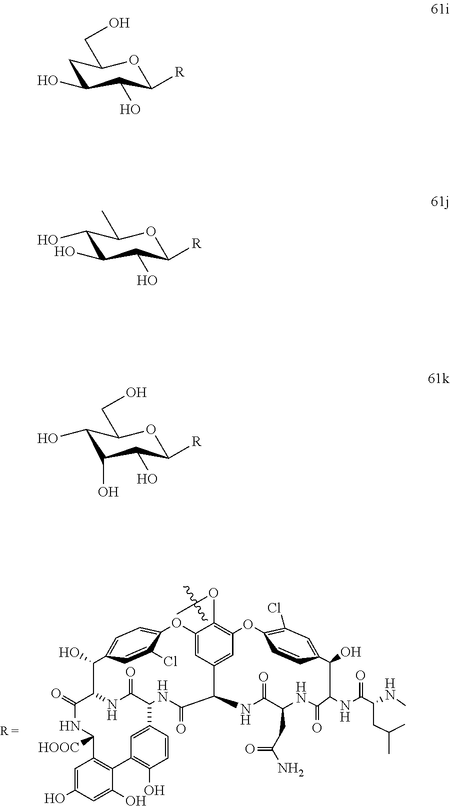

Evaluation of Single Enzyme Coupled System for Drug Screening.

Reactions containing 10.5 μM (50 μg) OleD variant TDP-16, 5 μM of UDP, 0.5 mM final acceptor (58 [as a positive control] and 62-111), and 0.5 mM 2-chloro-4-nitrophenyl-β-D-glucoside (9) in Tris-HCl buffer (50 mM, pH 8.5) with a final volume of 100 μl were prepared in a 96 well flat bottom Bacti plate (0.4 mL well⁻¹; Nagle Nunc International, Rochester, N.Y., USA). Absorbance measurements were recorded every 2 min at 410 nm for 8 hours on a FLUOstar Optima plate reader (BMG, Durham, N.C., USA) with the plate shaken within the reader for 5 seconds before collection of each time point. Reactions containing final acceptor were run at n=1, control reactions lacking final acceptor were run at n=6, and control reactions lacking both final acceptor and UDP were run at n=3. At 8 hours, reactions were filtered through a MultiScreen Filter Plate with a 10 kDa molecular weight cut-off (Millipore, Billerica, Mass., USA) according to manufacturer's instructions at 4° C., frozen at −20° C., and thawed for analysis.

‘Hits’ (62-103) based upon area under the curve were advanced for further confirmation via HPLC and/or LC-MS. LC/ESI-MS mass spectra of ‘hits’ were obtained using electrospray ionization in both (+) and (−) mode on an Agilent 1100 HPLC-MSD SL quadrupole mass spectrometer connected to a UV/Vis diode array detector. A 4.6 mm×2.0 mm C18 column Phenomenex, Torrance, Calif., USA) for separation with a gradient of 3% CH3CN (solvent B) for 1 min, 3% to 75% B over 8 min, 75% to 3% B over 1 min, 3% B for 1 min (A=ddH₂O; flow rate=1 mL min⁻¹; A₂₅₄ nm) were utilized for all analyses.

It should be noted that the above description, attached materials and their descriptions are intended to be illustrative and not limiting of this invention. Many themes and variations of this invention will be suggested to one skilled in this and, in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments. All publications, references to deposited sequences, patents and patent applications cited herein are hereby incorporated by reference in their entirety for all purposes.

What is claimed is:

1. An isolated mutant glycosyltransferase comprising:
(a) the amino acid sequence of OleD glycosyltransferase set forth in SEQ ID NO:1, wherein proline at position 67 has been replaced with threonine, serine at position 132 has been replaced with phenylalanine, alanine at position 242 has been replaced with leucine, and glutamine at position 268 has been replaced with valine; or
(b) an amino acid sequence substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine;
wherein said isolated mutant exhibits an improved conversion of nucleotide diphosphate (NDP) to NDP sugar as compared to a corresponding non-mutated glycosyltransferase.

2. The isolated mutant glycosyltransferase according to claim 1, wherein said isolated mutant glycosyltransferase is encoded by a nucleotide that hybridizes under stringent conditions to the nucleotide sequence set forth in SEQ ID NO:2.

3. A method of providing an isolated mutant glycosyltransferase with improved conversion of nucleotide diphosphate (NDP) to NDP sugar as compared to a corresponding non-mutated glycosyltransferase, comprising:
(a) mutating an isolated nucleic acid sequence encoding an amino acid sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine;
(b) expressing said isolated nucleic acid in a host cell; and
(c) isolating from said host cell a mutant glycosyltransferase that is characterized by improved conversion of nucleotide diphosphate (NDP) to NDP sugar as compared to a corresponding non-mutated glycosyltransferase.

4. A method of providing a nucleotide diphosphate (NDP) sugar, comprising incubating a nucleotide diphosphate and a glycoside donor in the presence of an isolated mutant glycosyltransferase according to claim 1 to provide an NDP sugar.

5. The method according to claim 4, wherein said glycoside donor has the structure: 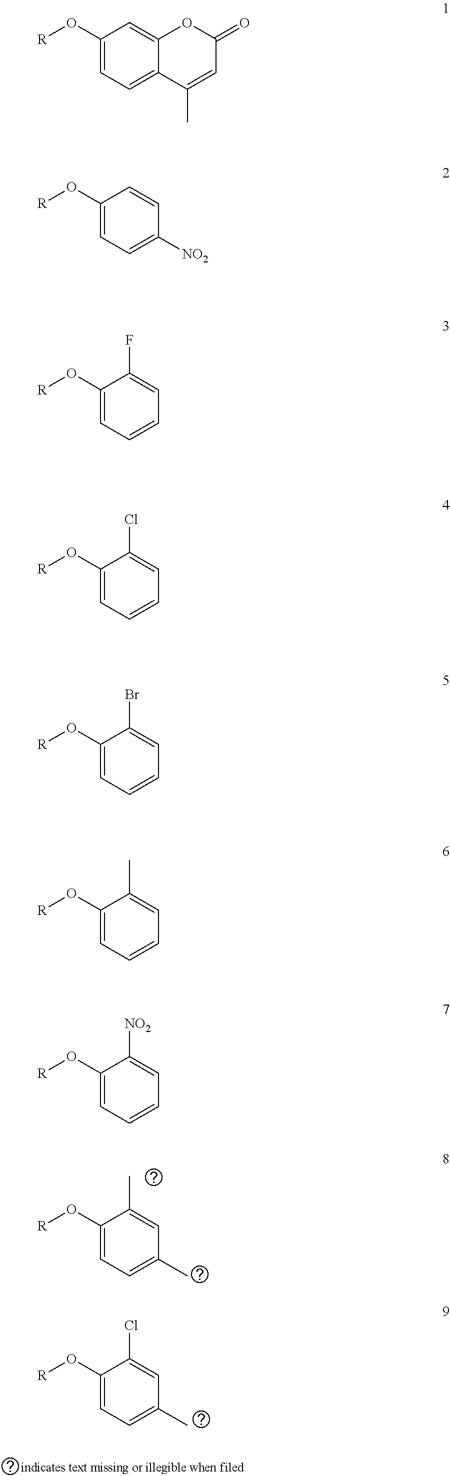
wherein R is β-D-glucopyranose.

6. The method according to claim 4, wherein the glycoside donor has the structure: 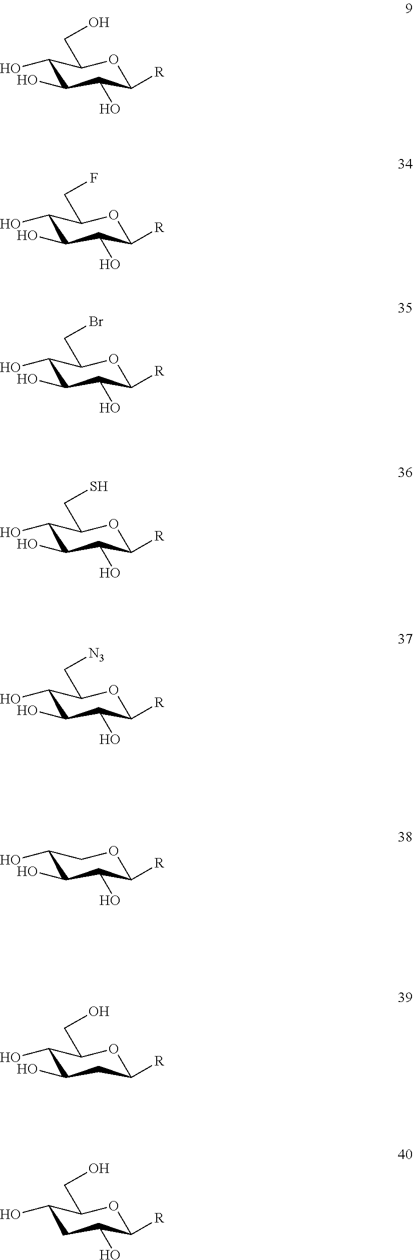 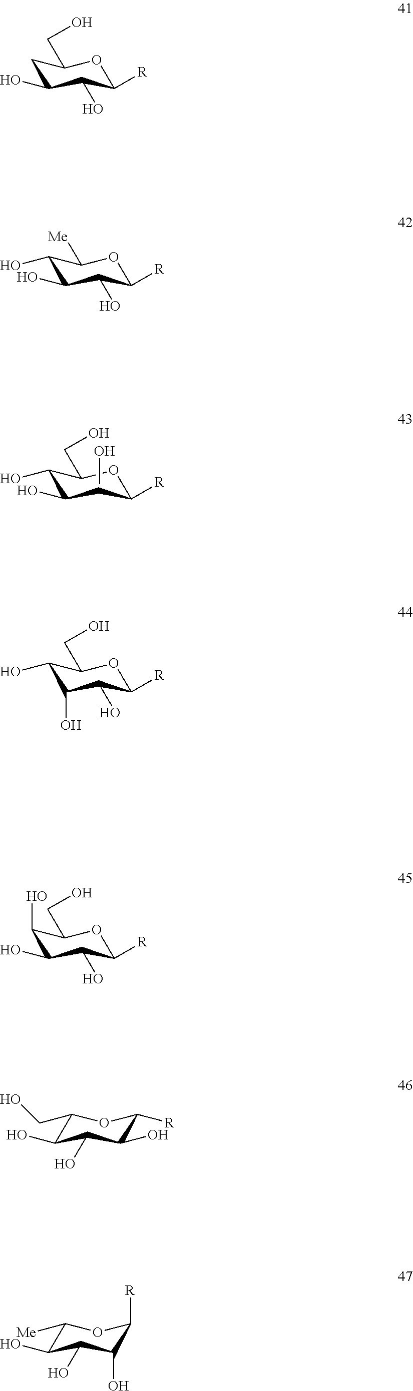 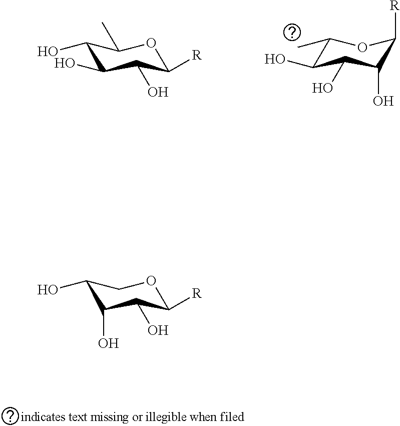
wherein R is:

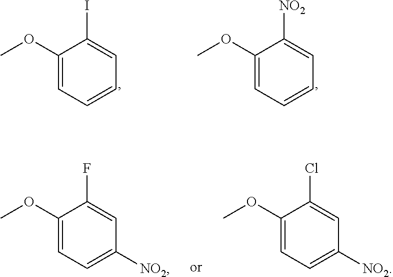

7. The method according to claim 4, wherein said NDP is uridine or thymidine diphosphate.

8. The method according to claim 4, wherein the NDP sugar includes a ¹³C atom.

9. A method of providing a glycosylated target molecule, comprising:
(a) incubating a nucleotide diphosphate and a glycoside donor in the presence of an isolated mutant glycosyltransferase according to claim 1 to provide a nucleotide diphosphate (NDP) sugar; and
(b) further incubating the NDP sugar with a second glycosyltransferase and a target molecule to provide a glycosylated target molecule.

10. The method according to claim 9, wherein said glycoside donor has the structure: 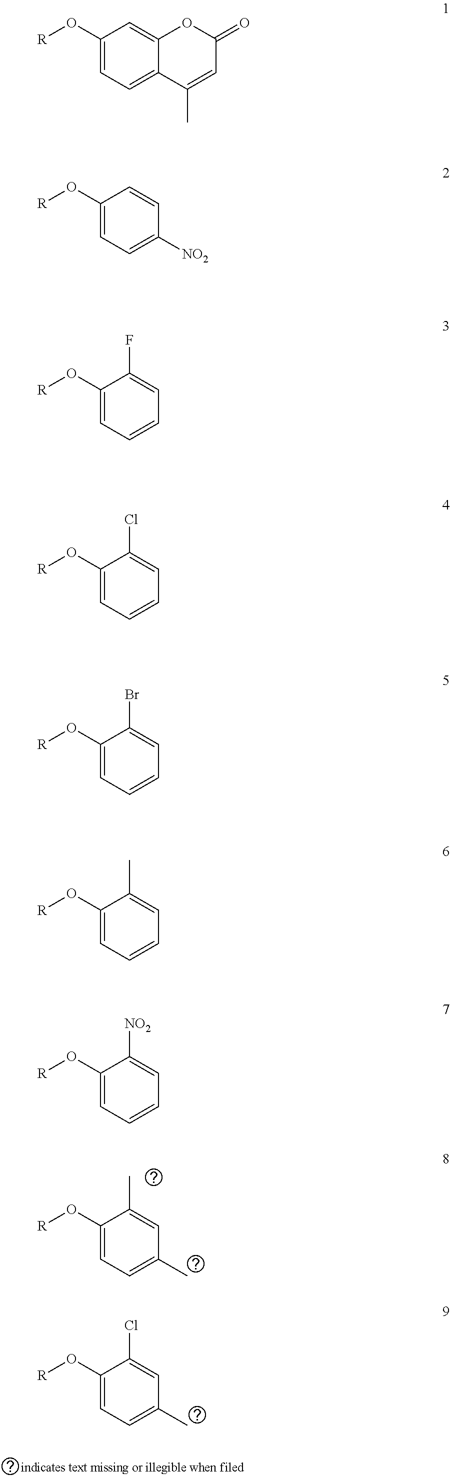
wherein R is β-D-glucopyranose.

11. The method according to claim 9, wherein the glycoside donor has the structure: 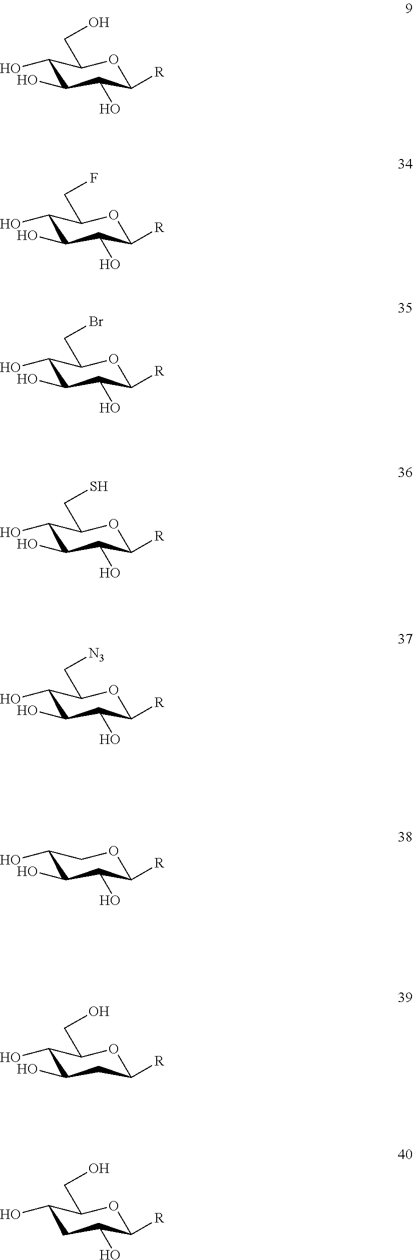 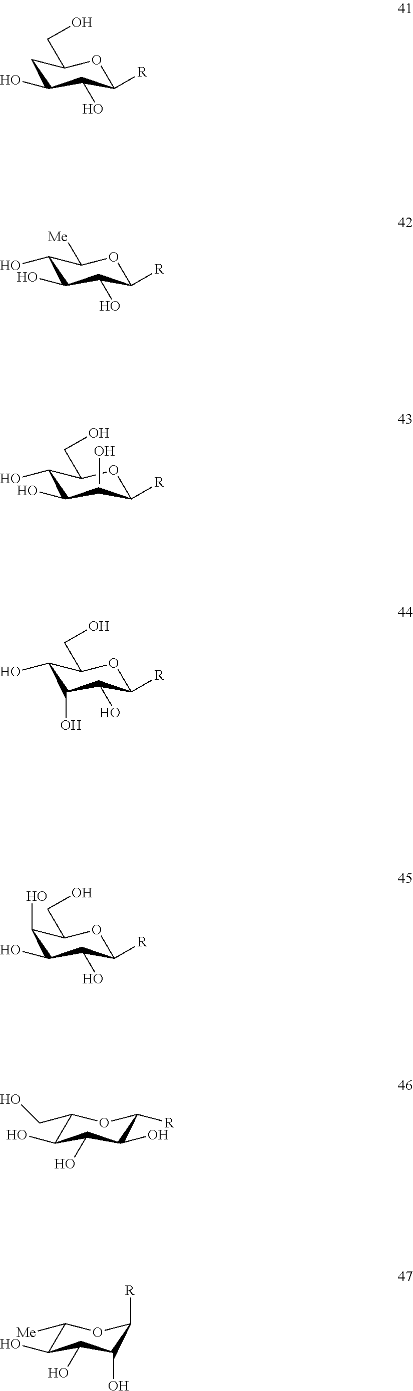 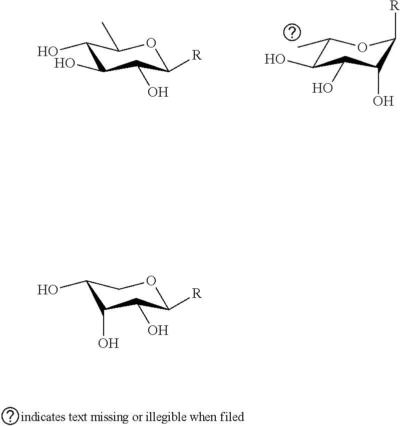
wherein R is:

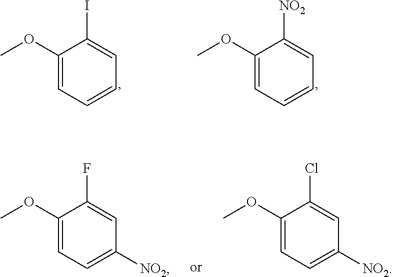

12. The method according to claim 9, wherein said NDP is uridine or thymidine diphosphate.

13. The method according to claim 9, wherein said target molecule is selected from the group consisting of natural or synthetic pyran rings, furan rings, enediynes, anthracyclines, angucyclines, aureolic acids, orthosomycins, macrolides, aminoglycosides, non-ribosomal peptides, polyenes, steroids, lipids, indolocarbazoles, bleomycins, amicetins, benzoisochromanequinones, flavonoids, isoflavones, coumarins, aminocoumarins, coumarin acids, polyketides, pluramycins, aminoglycosides, oligosaccharides, nucleosides, peptides and proteins.

14. The method according to claim 9, wherein the method is carried out in a single reaction vessel.

15. The method according to claim 9, wherein the method is carried out in vitro.

16. The method according to claim 9, wherein more than one type of target molecule is incubated with the second glycosyltransferase to produce a diverse population of glycosylated target molecules.

17. The method according to claim 9, wherein more than one type of NDP is incubated with the isolated mutant glycosyltransferase according to claim 1 to produce a diverse population of NDP sugars.

18. An isolated nucleic acid encoding a mutant glycosyltransferase having a polypeptide sequence identical to or substantially identical to OleD glycosyltransferase (SEQ ID NO:1) in which proline at position 67 has been replaced with threonine, serine at position 132 has been replaced with phenylalanine, alanine at position 242 has been replaced with leucine, and glutamine at position 268 has been replaced with valine, wherein said isolated mutant glycosyltransferase exhibits an improved conversion of nucleotide diphosphate (NDP) to NDP sugar as compared to a corresponding non-mutated glycosyltransferase.

19. The isolated nucleic acid according to claim 18, wherein said isolated nucleic acid hybridizes under stringent conditions to the nucleotide sequence set forth in SEQ ID NO:2.

20. A recombinant vector, comprising the isolated nucleic acid according to claim 18.

21. A host cell, comprising the isolated nucleic acid according to claim 18.

22. A fluorescent-based assay for identifying a mutant glycosyltransferase exhibiting an improved conversion of nucleotide diphosphate (NDP) to NDP sugar as compared to a corresponding non-mutated glycosyltransferase, comprising:
(a) providing a mutant glycosyltransferase;
(b) incubating the mutant glycosyltransferase with an NDP and a fluorescent glycoside donor; and
(c) measuring a change in fluorescence intensity of the fluorescent glycoside donor incubated with the mutant glyscosyltransferase, the mutant glycosyltransferase's ability to transfer a sugar from said fluorescent glycoside donor to the NDP to form an NDP sugar indicated by an increase in the fluorescence of the fluorescent glycoside donor incubated with the mutant glycosyltransferase;
wherein said mutant glycosyltransferase exhibits an improved conversion of NDP to NDP sugar by displaying an increase in said fluorescent glycoside donor fluorescence as compared to a corresponding non-mutated glycosyltransferase.

23. The assay according to claim 22, wherein said glycoside donor has the structure: 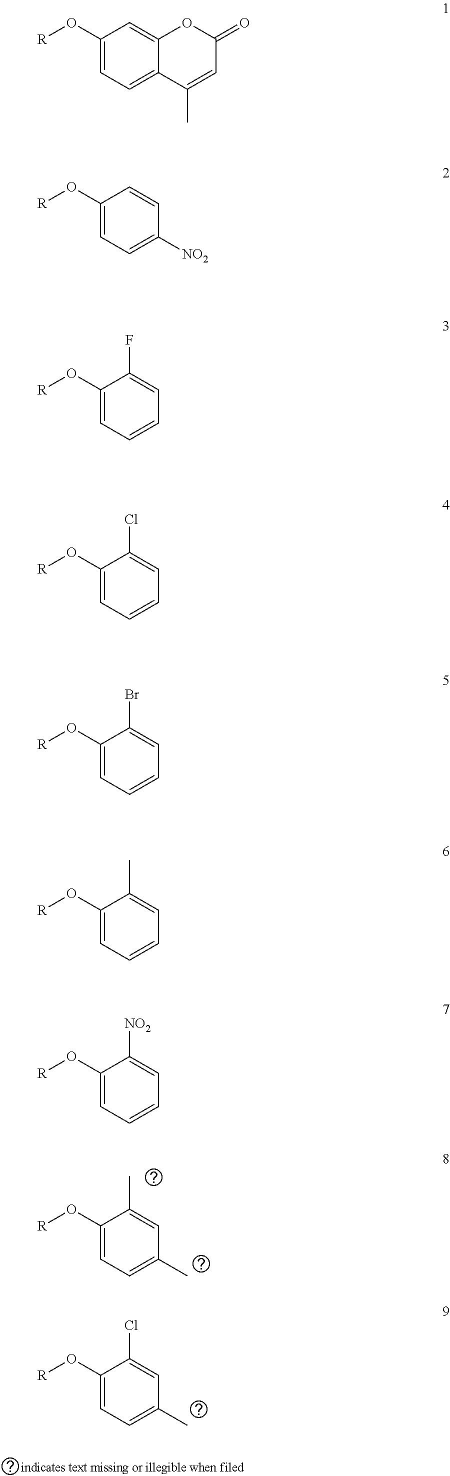
wherein R is β-D-glucopyranose.

24. The assay according to claim 22, wherein the glycoside donor has the structure: 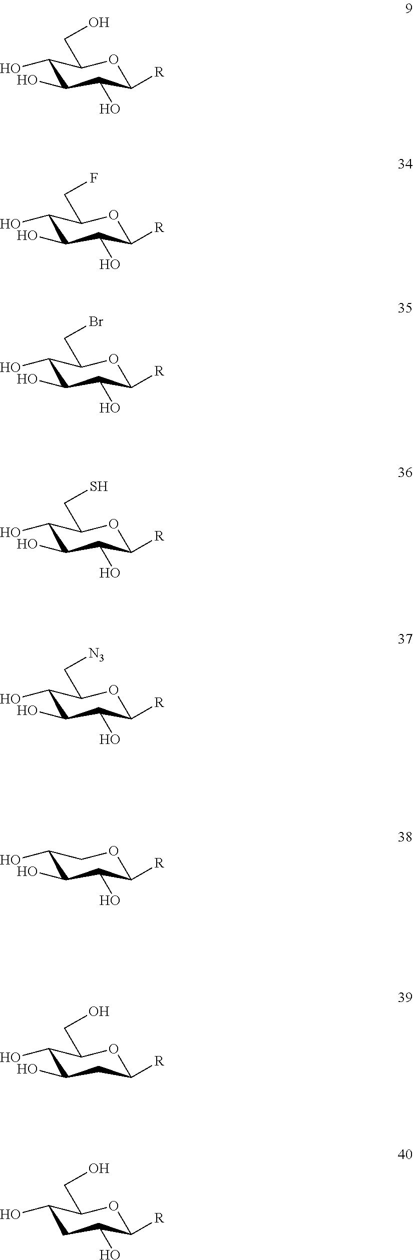 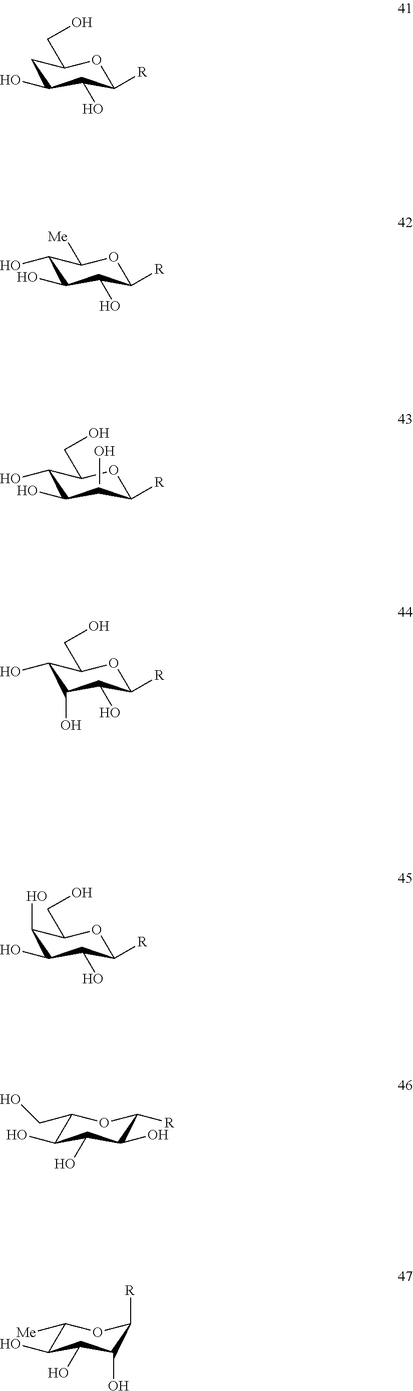 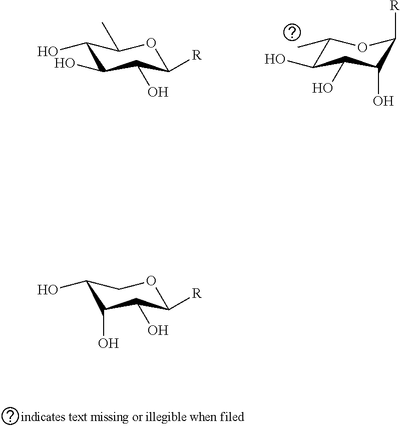
wherein R is:

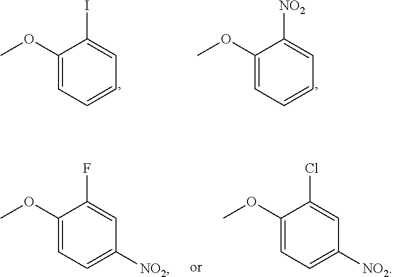

25. The assay according to claim 22, wherein said assay is carried out in parallel on a plurality of mutant glycosyltransferases.


SEQUENCE LISTING (2 sequences)

SEQ ID NO: 1 - protein, 415 aa, Streptomyces antibioticus
MTTQTTPAHIAMFSIAAHGHVNPSLEVIRELVARGHRVTYAIPPVFADKVAATGARPVLYHSTLPGPDADPEAWGSTLLDNVEPFLNDAIQALPQLADAYADDIPDLVLHDITSYPARVLARRWGVPAVSLSPNLVAWKGYEEEVAEPMWREPRQTERGRAYYARFEAWLKENGITEHPDTFASHPPRSLVLIPKALQPHADRVDEDVYTFVGACQGDRAEEGGWQRPAGAEKVVLVSLGSAFTKQPAFYRECVRAFGNLPGWHLVLQIGRKVTPAELGELPDNVEVHDWVPQLAILRQADLFVTHAGAGGSQEGLATATPMIAVPQAVDQFGNADMLQGLGVARKLATEEATADLLRETALALVDDPEVARRLRRIQAEMAQEGGTRRAADLIEAELPARHERQEPVGDRPNGG

SEQ ID NO: 2 - DNA, 1248 bp, Streptomyces antibioticus
atgaccacccagaccactcccgcccacatcgccatgttctccatcgccgcccacggccatgtgaaccccagcctggaggtgatccgtgaactcgtcgcccgcggccaccgggtcacgtacgccattccgcccgtcttcgccgacaaggtggccgccaccggcgcccggcccgtcctctaccactccaccctgcccggccccgacgccgacccggaggcatggggaagcaccctgctggacaacgtcgaaccgttcctgaacgacgcgatccaggcgctcccgcagctcgccgatgcctacgccgacgacatccccgatctcgtcctgcacgacatcacctcctacccggcccgcgtcctggcccgccgctggggcgtcccggcggtctccctctccccgaacctcgtcgcctggaagggttacgaggaggaggtcgccgagccgatgtggcgcgaaccccggcagaccgagcgcggacgggcctactacgcccggttcgaggcatggctgaaggagaacgggatcaccgagcacccggacacgttcgccagtcatccgccgcgctccctggtgctcatcccgaaggcgctccagccgcacgccgaccgggtggacgaagacgtgtacaccttcgtcggcgcctgccagggagaccgcgccgaggaaggcggctggcagcggcccgccggcgcggagaaggtcgtcctggtgtcgctcggctcggcgttcaccaagcagcccgccttctaccgggagtgcgtgcgcgccttcgggaacctgcccggctggcacctcgtcctccagatcggccggaaggtgacccccgccgaactgggggagctgccggacaacgtggaggtgcacgactgggtgccgcagctcgcgatcctgcgccaggccgatctgttcgtcacccacgcgggcgccggcggcagccaggaggggctggccaccgcgacgcccatgatcgccgtaccgcaggccgtcgaccagttcggcaacgccgacatgctccaagggctcggcgtcgcccggaagctggcgaccgaggaggccaccgccgacctgctccgcgagaccgccctcgctctggtggacgacccggaggtcgcgcgccggctccggcggatccaggcggagatggcccaggagggcggcacccggcgggcggccgacctcatcgaggccgaactgcccgcgcgccacgagcggcaggagccggtgggcgaccgacccaacggtgggtga